SEQUENCE LISTING (606 sequences)

SEQ ID NO: 1-17 - skipped (17 entries)

SEQ ID NO: 18 - protein, 32 aa, Rattus norvegicus
MLRGPGPGRLLLLAVLCLGTSVRCTETGKSKR

SEQ ID NO: 19 - protein, 38 aa, Rattus norvegicus
MLRGPGPGRLLLLAVLCLGTSVRCTETGKSKRLALQIV

SEQ ID NO: 20 - protein, 26 aa, Homo sapiens
MLRGPGPGLLLLAVQCLGTAVPSTGA

SEQ ID NO: 21 - protein, 31 aa, Xenopus laevis
MRRGALTGLLLVLCLSVVLRAAPSATSKKRR

SEQ ID NO: 22 - skipped

SEQ ID NO: 23 - DNA, 96 bp, Rattus norvegicus
  source 1..96: mol_type = genomic DNA
atgctcaggggtccgggacccgggcggctgctgctgctagcagtcctgtgcctggggacatcggtgcgctgcaccgaaaccgggaagagcaagagg

SEQ ID NO: 24 - DNA, 114 bp, Rattus norvegicus
  source 1..114: mol_type = genomic DNA
atgctcaggggtccgggacccgggcggctgctgctgctagcagtcctgtgcctggggacatcggtgcgctgcaccgaaaccgggaagagcaagaggcaggctcagcaaatcgtg

SEQ ID NO: 25 - DNA, 78 bp, Homo sapiens
  source 1..78: mol_type = genomic DNA
atgcttaggggtccggggcccgggctgctgctgctggccgtccagtgcctggggacagcggtgccctccacgggagcc

SEQ ID NO: 26 - DNA, 93 bp, Xenopus laevis
  source 1..93: mol_type = genomic DNA
atgcgccggggggccctgaccgggctgctcctggtcctgtgcctgagtgttgtgctacgtgcagccccctctgcaacaagcaagaagcgcagg

SEQ ID NO: 27 - protein, 17 aa, Rattus norvegicus
MTPLLLLAVLCLGTALA

SEQ ID NO: 28 - protein, 22 aa, Homo sapiens
MLSFVDTRTLLLLAVTLCLATC

SEQ ID NO: 29 - protein, 21 aa, synthetic construct
MWWRLWWLLLLLLLLWPMVWA

SEQ ID NO: 30-31 - skipped (2 entries)

SEQ ID NO: 32 - protein, 6 aa, synthetic construct
GGGGGP

SEQ ID NO: 33 - protein, 5 aa, synthetic construct
GGGGS

SEQ ID NO: 34 - protein, 5 aa, synthetic construct
EAAAK

SEQ ID NO: 35 - protein, 11 aa, synthetic construct
GGGTVGDDDDK

SEQ ID NO: 36 - DNA, 167 bp, synthetic construct
  source 1..167: mol_type = other DNA
aggaacccctagtgatggagttggccactccctctctgcgcgctcgctcgctcactgaggcaatttgataaaaatcgtcaaattataaacaggctttgcctgtttagcctcagtgagcgagcgagcgcgcagagagggagtggccaactccatcactaggggttcct

SEQ ID NO: 37 - DNA, 143 bp, synthetic construct
  source 1..143: mol_type = other DNA
aggaacccctagtgatggagttggccactccctctctgcgcgctcgctcgctcactgagggataaaaatccaggctttgcctgcctcagtgagcgagcgagcgcgcagagagggagtggccaactccatcactaggggttcct

SEQ ID NO: 38 - DNA, 143 bp, synthetic construct; identical to SEQ ID NO: 37
  source 1..143: mol_type = other DNA

SEQ ID NO: 39 - DNA, 208 bp, synthetic construct
  source 1..208: mol_type = other DNA
aggaacccctagtgatggagttggccactccctctctgggattgggattgcgcgctcgctcgcgggattgggattgggattgggattgggattgggattgataaaaatcaatcccaatcccaatcccaatcccaatcccaatcccgcgagcgagcgcgcaatcccaatcccagagagggagtggccaactccatcactaggggttcct

SEQ ID NO: 40 - DNA, 199 bp, synthetic construct
  source 1..199: mol_type = other DNA
aggaacccctagtgatggagttggccactccctctctgcgcgctcgctcgctcgggattgggattgggattgggattgggattgggattgataaaaatcaatcccaatcccaatcccaatcccaatcccaatcccgcgagcgagcgcgcaggagagggagtggccaactccatcactaggggttcctaagcttattata

SEQ ID NO: 41 - DNA, 154 bp, synthetic construct
  source 1..154: mol_type = other DNA
aggaacccctagtgatggagttggccactccctctctgcgcgctcgctcgctcactgagggcgcctataaagataaaaatccaggctttgcctgcctcagttagcgagcgagcgcgcagagagggagtggccaactccatcactaggggttcct

SEQ ID NO: 42 - DNA, 127 bp, synthetic construct
  source 1..127: mol_type = other DNA
ctagtgatggagttggccactccctctctgcgcgctcgctcgctcactgagggataaaaatccaggctttgcctgcctcagtgagcgagcgagcgcgcagagagggagtggccaactccatcactag

SEQ ID NO: 43-64 - skipped (22 entries)

SEQ ID NO: 65 - DNA, 20 bp, unidentified
  source 1..20: mol_type = unassigned DNA
cccagcccacttttccccaa

SEQ ID NO: 66 - protein, 6 aa, unidentified
PSPLFP

SEQ ID NO: 67-160 - skipped (94 entries)

SEQ ID NO: 161 - DNA, 145 bp, synthetic construct
  source 1..145: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcct

SEQ ID NO: 162-164 - skipped (3 entries)

SEQ ID NO: 165 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 166-174 - skipped (9 entries)

SEQ ID NO: 175 - protein, 27 aa, Homo sapiens
MGVRHPPCSHRLLAVCALVSLATAALL

SEQ ID NO: 176 - protein, 20 aa, Homo sapiens
MEFGLSWVFLVALLKGVQCE

SEQ ID NO: 177 - skipped

SEQ ID NO: 178 - protein, 20 aa, synthetic construct
MYRMQLLSCIALSLALVTNS

SEQ ID NO: 179 - skipped

SEQ ID NO: 180 - protein, 20 aa, synthetic construct
MYRMQLLLLIALSLALVTNS

SEQ ID NO: 181-445 - skipped (265 entries)

SEQ ID NO: 446 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 447 - DNA, 130 bp, synthetic construct
  source 1..130: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 448 - DNA, 145 bp, synthetic construct; identical to SEQ ID NO: 165
  source 1..145: mol_type = other DNA

SEQ ID NO: 449 - DNA, 136 bp, synthetic construct
  source 1..136: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagaggg

SEQ ID NO: 450-599 - skipped (150 entries)

SEQ ID NO: 600 - protein, 952 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPRVHSRAPSPLYSVEFSEEPFGVIVHRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWC

SEQ ID NO: 601 - DNA, 145 bp, synthetic construct
  source 1..145: mol_type = other DNA
tgggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 602 - DNA, 145 bp, synthetic construct
  source 1..145: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggacagatccg

SEQ ID NO: 603 - DNA, 738 bp, synthetic construct
  source 1..738: mol_type = other DNA
taaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcagcttacatgtg

SEQ ID NO: 604 - DNA, 2859 bp, Homo sapiens
  source 1..2859: mol_type = genomic DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 605 - DNA, 196 bp, Homo sapiens
  source 1..196: mol_type = genomic DNA
gatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttggg

SEQ ID NO: 606 - DNA, 4469 bp, synthetic construct
  source 1..4469: mol_type = other DNA
tgggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctggaggggtggagtcgtgacgtgaattacgtcatagggttagggaggtcggccgctctaggagttaatttttaaaaagcagtcaaaagtccaagtgcccttgcgagcatttactctctctgtttgctctggttaataatctcaggagcacaaacattccttactagttctaggagttaatttttaaaaagcagtcaaaagtccaagtgcccttgcgagcatttactctctctgtttgctctggttaataatctcaggagcacaaacattccttactagttctagagcggccgccagtgtgctggaattcggcttttttagggctggaagctacctttgacatcatttcctctgcgaatgcatgtataatttctacagaacctattagaaaggatcacccagcctctgcttttgtacaactttcccttaaaaaactgccaatcccactgctgtttggcccaatagtgagaactttttcctgctgcctcttggtgcttttgcctatggcccctattctgcctgctgaagacactcttgccagcatggacttaaacccctccagctctgacaatcctctttctcttttgttttacatgaagggtctggcagccaaagcaatcactcaaagttcaaaccttatcattttttgctttgttcctcttggccttggttttgtacatcagctttgaaaataccatcccagggttaatgctggggttaatttataactgagagtgctctagttctgcaatacaggacatgctataaaaatggaaagatgttgctttctgagagatcagcttacatgtggtaccgagctcggatccactagtaacggccgccagtgtgctggaattcggctttccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgagttgttgggattccaggcatcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggacagatccg

(19) United States
(12) Patent Application Publication
(10) Pub. No.: US 2025/0163457 A1
(43) Pub. Date: May 22, 2025

(54) THERAPEUTIC ADENO-ASSOCIATED VIRUS FOR TREATING POMPE DISEASE WITH LONG TERM CESSATION OF GAA ENZYME REPLACEMENT THERAPY

(71) Applicants: ASKLEPIOS BIOPHARMACEUTICAL, INC., Research Triangle Park, NC (US); DUKE UNIVERSITY, Durham, NC (US)

(72) Inventors: Sam HOPKINS, Research Triangle Park, NC (US); Edward Clinton SMITH, Durham, NC (US)

(21) Appl. No.: 18/840,822

(22) PCT Filed: Feb. 23, 2023
(86) PCT No.: PCT/US2023/013713

§ 371 Date: Aug. 22, 2024

Related U.S. Application Data
(60) Provisional application No. 63/314,093, filed on Feb. 25, 2022.
(60) Provisional application No. 63/319,034, filed on Mar. 11, 2022.
(60) Provisional application No. 63/340,972, filed on May 12, 2022.

(51) Int. Cl. C12N 15/86; A61K 38/47; A61K 48/00

(52) U.S. Cl. CPC C12N 15/86; A61K 38/47; A61K 48/0058; C12Y 302/0102; C12N 2750/14143

(57) ABSTRACT

Disclosed herein are methods for the treatment of Pompe Disease comprising administering a recombinant AAV (rAAV) vector comprising a rAVV genome comprising a heterologous nucleic acid encoding an acid alpha-glucosidase (GAA) polypeptide operatively linked to a liver-specific promoter, wherein the subject is withdrawn or not administered enzyme replacement therapy (ERT).

### RELATED APPLICATIONS

This application claims the benefit under 35 U.S.C. § 119(e) of U.S. Provisional Application Ser. No. 63/314,093, filed Feb. 25, 2022, and of U.S. Provisional Application Ser. No. 63/319,034, filed Mar. 11, 2022, and of U.S. Provisional Application Ser. No. 63/340,972, filed May 12, 2022, the contents of each of which are incorporated herein by reference in their entirety.

### FIELD OF THE INVENTION

The present invention relates to methods to treat Pompe disease by administering adeno-associated virus (AAV) particles, virions and vectors for expression of an alpha-glucosidase (GAA) polypeptide, where the method enables the ability to reduce, or eliminate the clinical need for administration of long-term GAA enzyme replacement therapy (ERT) for an extended period of time.

### BACKGROUND

Pompe disease (Glycogen storage disease type II; acid maltase deficiency; MIM 232300) is caused by recessive mutations of the GAA gene leading to complete or partial deficiency of the lysosomal enzyme acid α-glucosidase (GAA). Absence of GAA leads to the progressive accumulation of glycogen in the lysosomes of many tissues, particularly skeletal muscle and cardiomyocytes. Impaired energy metabolism then leads secondarily to severely disrupted muscle architecture, dysfunction, autophagy, and in adults, significant fatty replacement of skeletal muscle myocytes.

Clinically, the condition ranges from a fulminant infantile-onset Pompe disease (IOPD) typically leading to death before 12 months of age to a late-onset Pompe disease (LOPD), which is slowly progressive leading to myopathy causing loss of mobility and typically death from respiratory failure 5-15 years after diagnosis. Infantile-onset patients have cardiomyopathy often noted even at birth or even antenatally, with elevated creatine kinase (CK) and then within weeks to the first months of life develop severe hypotonia, respiratory insufficiency requiring ventilator support and massive cardiomegaly. Deaths are most often the result of cardiorespiratory failure, aspiration pneumonia or ventricular arrhythmias. Late-onset Pompe Disease (LOPD) patients (mostly adults, some juveniles) experience slowly progressive muscle weakness often leading to delayed diagnosis, extensive fatty replacement of trunk and proximal limb muscles, progressing to respiratory failure which is the primary cause of death (Carlier et al. 2011). Basilar artery aneurysms occur and can be life threatening if they rupture (El-Gharbawy et al. 2011; Hobson-Webb et al. 2012). As an alternative or adjunct to enzyme therapy, the feasibility of gene therapy approaches to treat GSD-II have been investigated (Amalfitano, A., et al., (1999) Proc. Natl. Acad. Sci. USA 96:8861-8866, Ding, E., et al. (2002) Mol. Ther. 5:436-446, Fraites, T. J., et al., (2002) Mol. Ther. 5:571-578, Tsujino, S., et al. (1998) Hum. Gene Ther. 9:1609-1616).

MYOZYME® (alglucosidase alfa) was the first US approved product (2006) for the treatment of Pompe disease; LUMIZYME® (alglucosidase alfa) was approved in 2010 and is the current standard-of-care (SOC) treatment for infantile-onset and late-onset Pompe patients. Alglucosidase alfa is administered intravenously every 2 weeks as an infusion at a dose of 20 mg/Kg (LUMIZYME Prescribing Information 2014). Alglucosidase alfa provides an exogenous source of GAA. Binding to mannose-6-phosphate receptors on the cell surface has been shown to occur via carbohydrate groups on the GAA molecule, after which it is internalized and transported into lysosomes, where it undergoes proteolytic cleavage that results in increased enzymatic activity. It then exerts enzymatic activity in cleaving glycogen. Although this enzyme replacement therapy (ERT) prolongs survival in most patients with infantile Pompe disease, a subset have either died, suffered ongoing muscle weakness or remained profoundly hypotonic requiring mechanical ventilator support despite compliance with SOC ERT. For late-onset patients, ERT moderately improves muscle function and pulmonary function parameters, initially, followed by stable function or decline and remains far from an ideal therapy (Schoser et al. 2017).

For infantile-onset patients, especially those with severe or null mutations (cross-reacting immune material [CRIM])-negatives), high and sustained anti-rhGAA Immunoglobulin G (IgG)-mediated immunity against the GAA enzyme is a primary reason for impaired or inadequate response to ERT. That is, ERT is known to provoke an antibody response in the form of both IgG and IgE and can also lead to infusion-associated reactions (Kishnani et al. 2007; Kishnani et al. 2010). Current practice is to initiate immune modulation with ERT for patients with LOPD at risk for antibody formation.

Additionally, enzyme replacement therapy (ERT) with alglucosidase alfa (MYOZYME®/LUMIZYME®) is delivered as an onerous every other week, or weekly infusion, and is the only treatment currently available. For those with infantile-onset Pompe disease (IOPD), GAA is absent (CRIM negative) or minimal (˜1% of normal) and causes rapidly progressive cardiorespiratory failure and death by the age of 2 years if left untreated (Parini et al. 2018). Subjects with a marked deficiency of GAA present as juveniles or adults (Late-Onset Pompe disease [LOPD]) with less severe symptoms and slower progression. Moreover, missing biweekly treatments can result in significant set backs requiring many months of ERT to return to the same levels.

Accordingly, despite temporary therapeutic success, alglucosidase alfa ERT leaves a clear unmet medical need in both IOPD and LOPD. Longitudinal data in subjects confirm that ERT does not lead to complete correction or normalization of patients with Pompe disease. Ultimately subjects typically still decline, albeit at a slower rate, delaying the inevitable progression to death (Kuperus et al. 2017; Parini et al. 2018). While alglucosidase alfa prolongs survival for subjects with both IOPD and LOPD (LUMIZYME Prescribing Information, 2014) the antibody responses to the GAA and decline in effect poses several drawbacks.

Therefore, from a clinical perspective, long-term treatment of Pompe patients with ERT has had limited success. In the IOPD subset of patients, many experience high, sustained anti-rhGAA antibody titers (HSAT). Pompe patients who lack any residual GAA protein are deemed CRIM-negative. CRIM negative patients develop HSAT, and a subset of CRIM positive patients who also develop high or sustained intermediate titers suffer greatly increased mortality (Banugaria et al. 2011). Furthermore, the use of immunosuppression to prevent antibody formation in patients at risk for HSAT significantly prolonged survival and confirmed the clinical relevance of HSAT (Mendelsohn et al. 2009; Banugaria et al. 2011). Moreover, literature indicates that only about ˜1% of ERT is pharmacologically active.

Therefore, while enzyme therapy has demonstrated reasonable efficacy for severe infantile GSD II, the benefit of GAA enzyme therapy is limited by the need for frequent infusions as well as the subject developing inhibitor or neutralizing antibodies against recombinant hGAA protein (Amalfitano, A., et al. (2001) Genet. In Med. 3:132-138), there is a need for improved methods and alternative therapies to treat patients with Pompe disease. Adeno-associated virus (AAV) vector-mediated gene transfer provides an appropriate and feasible alternative.

### SUMMARY OF THE INVENTION

The technology described herein relates generally to gene therapy constructs, methods and composition, for the treatment of Pompe Disease. More particularly, the technology relates to methods of using adeno-associated (AAV) virions configured for delivering a heterologous nucleic acid encoding GAA polypeptide to a subject, and more particularly for delivering a heterologous nucleic acid encoding GAA polypeptide to the liver of a subject where the GAA polypeptide is secreted from the liver cells.

In particular, the technology described herein is based on the discovery from a human clinical trial which demonstrates the ability to reduce, or eliminate the clinical need for long-term hGAA ERT administration for an extended period of time when the subject is administered a AAV expressing GAA polypeptide.

Without wishing to be bound by theory, the only current treatment for Pompe disease is long-term administration of recombinant human GAA (rhGAA) ERT, which is typically administered on an every-other week regimen. Herein, the inventors have demonstrated that a subject with Pompe disease can take breaks from the normal ERT regimen for extended period of time (e.g., extended periods of ERT cessation) without a clinical set back if the subject is administered a specific dose of AAV vector expressing a GAA polypeptide as disclosed herein. In some embodiments, withdrawal of the administration of long-term ERT begins at about the time of administration of the AAV vector to the subject (e.g., the day before, the day of, or the day after), or in some embodiments, withdrawal of the administration of long-term ERT can occur at about 24 weeks, or anywhere within about 24 to about 26 weeks after administration of the AAV vector.

In some embodiments, a subject administered a AAV vector expressing GAA as disclosed herein, can, after an initial period of withdrawal of the administration of long-term ERT for an extended period of time, be administered complementary ERT, where the complementary ERT is administered after about 6-months, or about 1 year, or longer than a year of cessation of the long-term ERT. Stated differently and without wishing to be bound by theory, the technology disclosed herein relates to a method whereby a subject with Pompe disease who is administered a AAV vector expressing GAA as disclosed herein, can have breaks or “holidays” from the normal long-term ERT administration. That is, according to the methods as disclosed herein, a subject administered an AAV vector expressing GAA as disclosed herein can have extended periods of time with the absence of administration of long-term ERT administration. In some embodiments, the methods as disclosed herein enable flexibility in normal ERT regimens, in that extended breaks or withdrawal of administration of long-term ERT does not result in a clinical decline—that is, a subject remains clinically stable despite not having ongoing long-term ERT.

In some embodiments, the methods as disclosed herein encompass re-administration of ERT (herein referred to as “complementary ERT”) after an extended period of time of cessation of ERT administration, and enable flexibility in normal ERT regimen, as the continued production of GAA expressed by the AAV permits ERT flexibility. In some embodiments, the complementary ERT is pulse administration of ERT, as disclosed herein. In some embodiments, the complementary ERT is at less frequent intervals, or at a lower dose, or at irregular doses, or at irregular intervals as compared to the prior administration of long-term ERT.

Accordingly, the methods as disclosed herein provide significant advantages to subjects with Pompe disease, including but not limited to reducing or eliminating the rigorous and arduous weekly, or every-other week infusions of long-term rhGAA ERT treatment, which are significantly time-consuming and geographically limiting, and hinders a patient with Pompe disease from travelling for prolonged periods from areas where their ERT infusions are administered. Additionally, as disclosed herein, the absence of ERT administration also reduces any side effects due to anti-rhGAA antibodies against the ERT, and also for some doses circumvents the need for administration of immune suppressants normally co-administered with the ERT. As such, the methods to treat Pompe disease as disclosed here leads to greater flexibility in Pompe treatment and an improvement in quality of life and lifestyle of subjects with Pompe disease.

Accordingly, in one embodiment, the technology relates to a method of treating Pompe disease in a subject, comprising administering to the subject a pharmaceutical composition comprising a recombinant adeno-associated virus (AAV) vector comprising in its genome, a heterologous nucleic acid sequence encoding an alpha-glucosidase (GAA) polypeptide in expressible form wherein the heterologous nucleic acid is operatively linked to a liver-specific promoter, in the absence of administration of long-term GAA enzyme replacement therapy (ERT) for an extended period of time (e.g., ERT administration can be withdrawn or stopped at about 24 weeks, or at about 26 weeks, or earlier than 24- or 26 weeks, e.g. anytime between a day and 26 weeks, after administration of the recombinant AAV). In some embodiments, the dosage of the recombinant AAV comprising nucleic acid encoding GAA is no more than 4.0E¹² vg/kg, and in some embodiments, the GAA is expressed to a level that the subject obtains a blood serum level of GAA expressed by the AAV at a pharmaceutical activity range from 160 to ≤2,260 nmol/mL/hr, 165 to ≤2,260 nmol/ml/hr, 175 to ≤2,260, 180 to ≤2,260, 185 to ≤2,260, 189 to ≤2,260 of at least within two weeks of administration. In some embodiments, the dosage of the AAV expressing GAA is no more than 4.0E¹² vg/kg, and in some embodiments, the GAA is expressed to a level that the subject obtains a blood serum level of GAA expressed by the AAV at a pharmaceutical activity range from 189 to ≤2,260 nmol/mL/hr of at least within two weeks of administration. In some embodiments, the dosage of the AAV expressing GAA is no more than 4.0E¹² vg/kg, and in some embodiments, the GAA is expressed to a level that the subject obtains a blood serum level of GAA expressed by the AAV at a pharmaceutical activity range from 189 to ≤2,260 nmol/mL/hr of at least within two weeks of administration. In some embodiments the blood serum levels of GAA is obtained within at least about 5 days, 6, days, 7 days, 8, days, 9 days, 10 days, 11 days, 12 days, 13 days, 14 days, or 15 days. In some embodiments the blood serum level of GAA is obtained within about 3 weeks, about 4 weeks, about 5 weeks, or later. In some embodiments, the specified blood serum levels is attained in this time frame as a result of a dose of AAV-GAA of between 5e¹⁰ vg/kg to 1.6e¹³ vg/kg. In some embodiments, the subject has IOPD. In some embodiments, the subject has LOPD.

In some embodiments; the dosage of the recombinant AAV comprising nucleic acid encoding GAA is between about 1.6E12 to about 1.6E13 vg/kg; and the GAA is expressed to a level that the subject obtains a blood serum level of GAA expressed by the AAV at a pharmaceutical activity range from 160 to ≤2,260 nmol/mL/hr, 165 to ≤2,260 nmol/ml/hr, 175 to ≤2,260, 180 to ≤2,260, 185 to ≤2,260, 189 to ≤2,260 of at least within two weeks of administration, or within less than two weeks of administration. In some embodiments of the invention, the glycogen content in tissue is reduced from baseline by about 10% to about 40%, about 10% to about 30%, or, about 15% to about 25% after rAAV administration. In some embodiments, after rAAV administration, clinical stable motor function is observed, as determined by the 6MWT is ≤12% decline, or less than a 43-meter decrease from baseline in two consecutive assessments no less than 3-months apart. Stated differently, a clinical stable level of motor function as determined by the 6MWT position is within a 0-12% decline from a baseline level in two consecutive assessments no less than 3-months apart. In some embodiments, after rAAV administration, a clinical stable FVC % is observed as predicted in an upright position is ≤15% decrease from baseline in two consecutive assessments no less than 3-months apart. Stated differently, a clinical stable level of pulmonary function as determined by the % FVC predicted in an upright position is between 1-14% from a baseline in two consecutive assessments no less than 3-months apart.

In some aspects of the invention described herein, immune modulators is coadministered with AAV on the day of AAV administration or, any day between day 1-15 days prior to AAV administration. In some embodiments, the immune modulators are steroidal, and/or, non-steroidal; and/or, a combination thereof. In some embodiments, the immune modulator is prednisone. In some embodiment, the immune modulator is methotrexate. In some embodiments, the immune modulator is a combination of methotrexate and prednisone. In some embodiments, methotrexate is administered at an initial dose between about 2.5 mg/day to about 30 mg/day. In some embodiment, prednisone is administered at an initial dose of 60 mg/day with subsequent tapering to lower doses.

In some embodiments of the invention, the dosage of the recombinant AAV (rAAV) comprising nucleic acid encoding GAA is between about 1.6E10 to about 1.6E12 vg/kg, where the subject receiving the said AAV doses received a prior ERT and where the ERT is withdrawn on the same day, a day after, a day before or, anytime between day 1 and week 26 after the rAAV is administered on the day of ERT withdrawal, a day after, a day before or, anytime between a day; and the GAA is expressed to a level that the subject obtains a blood serum level of GAA expressed by the AAV at a pharmaceutical activity range from 160 to ≤2,260 nmol/mL/hr, 165 to ≤2,260 nmol/ml/hr, 175 to ≤2,260, 180 to ≤2,260, 185 to ≤2,260, 189 to ≤2,260 of at least within two weeks of administration, or, within less than two weeks of administration. In some embodiments, ERT is withdrawn after a longer timepoint, e.g, after more than about 26 weeks. In some aspects of the invention described herein, immune modulators is coadministered with AAV on the day of AAV administration or, any day between day 1-15 days prior to AAV administration. In some embodiments, the immune modulators are steroidal, and/or, non-steroidal; and/or, a combination thereof. In some embodiments, the immune modulator is prednisone. In some embodiment, the immunomodulator is methotrexate. In some embodiments, the immunomodulator is a combination of methotrexate and prednisone. In some embodiments, methotrexate is administered at an initial dose between about 2.5 mg/day to about 30 mg/day. In some embodiments, prednisone is administered at an initial dose of 60 mg/day with subsequent tapering to lower doses.

In some aspects of the invention, there is no substantial overlap between the rAAV administration and ERT. In some aspects of the invention, there is only a day of overlap between the rAAV administration and ERT. In some embodiments, the subject receives a single rAAV infusion and the prior ERT was withdrawn prior to or, on the same day of rAAV infusion. In some embodiments, the subject receives prior ERT than rAAV administration for at least about 24 weeks, at least about 26 weeks, at least about 30 weeks, at least about 50 weeks, about 52 weeks or, more. After the ERT is withdrawn, single rAAV administration of at least 1.6E12 vg/kg can produce one or, more of clinical stable outcome as measured by GAA level in the normal range in circulation, and tissue, Glycogen level within normal range in tissue, clinical stable FVC %, clinical stable 6MWT.

In particular, the technology described herein relates to the discovery that a single infusion of a rAAV vector expressing human acid alpha-glucosidase (GAA) can be a stand-alone replacement for repeated infusions of enzyme replacement therapy (ERT) with recombinant human GAA protein (rhGAA). The inventors demonstrate that a one-time administration of AAV expressing GAA leads to long-term transduction of a normal GAA gene into hepatocytes and continuous constitutive expression of GAA in the systemic circulation. Accordingly, the inventors demonstrate herein that administration of a composition comprising AAV expressing hGAA can replace the biweekly exogenous administration of ERT that subjects with Pompe disease normally receive. That is, the inventors have demonstrated herein that subjects with Pompe that are administered a AAV expressing hGAA as disclosed herein can have long term cessation of ERT.

In particular, described herein is a method of treating Pompe in a subject in need thereof by administering the subject a composition comprising a AAV vector expressing the α-glucosidase (GAA) protein, where the subject is not being concurrently administered a GAA enzyme replacement therapy. In some embodiments, the technology relates to a method of administering a AAV expressing GAA where the subject can be withdrawn from a GAA enzyme replacement therapy (ERT) for an extended period of time, e.g., at least 3 months, at least 4 months, at least 5 months, at least 1 year, at least 1½ years and points in between 6 months or longer. In some embodiments, the subject is withdrawn from ERT on the day of, or shortly before administration of a AAV expressing GAA, and is clinically stable with respect to at least one or more, as disclosed herein. In some embodiments, the subject is withdrawn from ERT at any time between 1-2 days before or after administration, and about 6-months after administration of a AAV expressing GAA, and is clinically stable with respect to at least one or more Pompe symptoms for at least 6 months, as disclosed herein.

Additionally, the inventors have also discovered that Pompe patients administered a AAV expressing GAA according to the methods and dose ranges as disclosed herein, there is minimal immune response to the GAA protein expressed by the AAV. According, in some embodiments, there is minimal, or no need for immune modulation or administration of immune suppressants at the time of, or before, or after the administration of the AAV to the subject, and therefore normal immune suppressants protocols which are typically administered when a subject is administered a viral vector, or undergoing gene therapy are not required.

Accordingly, in all embodiments herein, the method to treat Pompe comprises, or consists essentially of, or consists of, administering an AAV vector expressing GAA as disclosed herein, in the absence of administration of ERT for Pompe, and also in the absence of immune modulation. In some embodiments, the subject has late onset Pompe Disease (LOPD) or infantile-onset Pompe disease (IOPD).

In all aspects disclosed herein, the AAV comprises a nucleotide sequence containing inverted terminal repeats (ITRs), a promoter, a heterologous gene, a poly-A tail and potentially other regulator elements for use to treat a Pompe disease, e.g., late onset Pompe disease (LOPD), wherein the heterologous gene is GAA, and wherein the vector, e.g., rAAV can be administered to a patient in a therapeutically effective dose that is delivered to the appropriate tissue and/or organ for expression of the heterologous GAA gene and treatment of the disease, e.g., Pompe disease.

Aspects of the present invention teach certain benefits in construction and use which give rise to the exemplary advantages described below. Other features and advantages of aspects of the present invention will become apparent from the following more detailed description, taken in conjunction with the accompanying drawings, which illustrate, by way of example, the principles of aspects of the invention.

Aspects of the invention described herein also relate to a method of treating Pompe disease in a subject, comprising administering to a subject who is being treated for Pompe disease with long-term GAA enzyme replacement therapy (ERT), a pharmaceutical composition comprising a recombinant adeno-associated virus (AAV) vector comprising in its genome, a heterologous nucleic acid sequence encoding a polypeptide comprising an alpha-glucosidase (GAA) polypeptide in expressible form at a dosage of between about 1.6 e¹² vg/kg to about 1.6e¹³ vg/kg, wherein the heterologous nucleic acid is operatively linked to a liver-specific promoter, and wherein the administration of long-term GAA enzyme replacement therapy (ERT) is withdrawn on the same day (d1), the day after, or at least the day before the administration of the recombinant AAV, and wherein the subject obtains a blood serum level of GAA expressed by the AAV at a pharmacological activity is at least 165 nmol/ml/hr of at least within two weeks of administration. In some embodiments of the methods disclosed herein, the subject is administered methotrexate and prednisone for immune modulation. In some embodiments of the methods disclosed herein, methotrexate is administered at an initial dose of 30 mg or less/week, between about 5 and 30 mg/week, or between 5 and 7.5 mg/week.

### BRIEF DESCRIPTION OF THE DRAWINGS

The accompanying drawings illustrate aspects of the present invention. In such drawings:

FIG. 1 is an illustration of a plasmid map of pAAV-LSPhGAA plasmid, used to generate AAV8-hGAA (ACTUS-101) for use in the methods to treat Pompe disease in accordance with at least one embodiment. The nucleotide sequences of the ITR's (single underlined), promoter (double underlined), the coding sequence of the hGAA (capital letters), and the polyA (bold and italicized) are also shown. The entire sequence shown is SEQ ID NO: 606. The 5′ ITR sequence is SEQ ID NO: 601, the 3′ ITR sequence is SEQ ID NO: 602, the promoter sequence is SEQ ID NO: 603, the coding sequence for the hGAA is SEQ ID NO: 604, and the polyA sequence is SEQ ID NO: 605.

FIG. 2A and FIG. 2B are tables (A) and (B) that each show the initial demographics and baseline characteristics of the cohort.

FIG. 3 contains two tables, the upper table shows adverse events and the lower table shows adverse events reported and the reports for resolve for the respective subjects in the cohort through week 52.

FIG. 4 is a series of graphical representations of safety and efficacy data taken for each subject (101001, 101002, and 101003) over the indicated period of time. ELIspot activity was taken using 3 polypeptides representing the AAV8 capsid protein, AAV8_A, AAV8_B, and AAV8_C. Levels of serum GAA and alanine aminotransferase (ALT) are also shown.

FIG. 5A-FIG. 5D is a series of graphical representations of bioactivity markers. From left to right, FIG. 5A) Serum GAA, FIG. 5B) muscle GAA, FIG. 5C) muscle glycogen and FIG. 5D) glucose tetrasaccharide (Glc4).

FIG. 6 is a set of graphical representations of data from measurements of muscle function.

FIG. 7 is a set of graphical representations of data from measurements of muscle fatigue.

FIG. 8 is Appendix 1.

FIG. 9 is Appendix 2.

FIG. 10A-FIG. 10F is a series of graphical representations of data showing a 52-Week Safety Profile of Liver Transaminases, CK, Anti-rhGAA Ab and AAV8-A ELISPOT. FIG. 10A: Aspartate Aminotransferase; FIG. 10B: Alanine Aminotransferase; FIG. 10C: Gamma Glutamyl Transferase; FIG. 10D: Creatine Kinase; FIG. 10E: Anti-Human Recombinant Acid Alpha-Glucosidase Antibody (Inverse titer); FIG. 10F: T Cell Response (AAV8-A).

FIG. 11 is a graphical representation of Serum Acid Alpha-glucosidase activity of each patient over 52 weeks.

FIGS. 12A-12B show the results of long term follow-up of each subject (001, 002, and 003) at 104 weeks post administration of AAV8-LSPhGAA (ACTUS 101). FIG. 12A shows the results from the 6MWT (6-minute walk test), and FIG. 12B shows the results of the FVC (Forced vital capacity). Subjects 001 and 002 remained without ERT for the entire period of 24-104 weeks, and subject 003 withdrew from ERT at 24 weeks but resumed ERT after 97 weeks.

FIG. 13A-13B is a graphical representation of the T cell reactivity to the AAV8 vector (SFU/million PBMCs), and transaminitis (ALT level), and serum GAA levels in subjects from cohort 2. FIG. 13A is data for subject 006, and FIG. 13B is data for subject 004.

FIG. 14 is a graphical representation of the regimen of methotrexate and prednisone for the subject of cohort 3.

FIG. 15 is a table showing biopsy results for the subject of cohort 3, at 24 weeks and 52 weeks.

FIG. 16 is a graphical representation of results of cohort 3 showing T cell reactivity to vector capsid and GAA as indicated by SFU/1e6 PBMCs (bars), and also of transaminitis as indicted by AST/ALT (IU/L) level (solid lines). The dashed lines indicate the normal range of ALT (top line) and the normal range of AST (bottom line).

FIG. 17 is a graphical representation of Cohort 3 serum GAA levels over time.

FIG. 18 is a schematic showing the design of the study. Supplies are prepared for Treatment A (active and indistinguishable placebo) and for Treatment B (active and indistinguishable placebo) and subjects then take two sets of treatment; either A (active) and B (placebo), or A (placebo) and B (active).

The above described figures illustrate aspects of the invention in at least one of its exemplary embodiments, which are further defined in detail in the following description. Features, elements, and aspects of the invention that are referenced by the same numerals in different figures represent the same, equivalent, or similar features, elements, or aspects, in accordance with one or more embodiments.

### DETAILED DESCRIPTION

The technology described herein relates to the discovery that administration of a rAAV vector expressing human GAA can be a stand-alone replacement for repeated infusions of GAA enzyme replacement therapy (ERT). Herein in the Examples demonstrate that a single administration of AAV expressing GAA as disclosed herein at an appropriate dose leads to permanent transduction of a normal GAA gene into hepatocytes and continuous constitutive expression of systemic GAA, negating the need for weekly or every other week exogenous administration of ERT that subjects with Pompe disease normally receive. That is, the inventors have demonstrated herein that subjects with Pompe disease that are administered a AAV expressing hGAA as disclosed herein can have long term cessation of ERT.

Accordingly, the disclosure described herein generally relates to methods and compositions to treat Pompe Disease in a subject, the method comprising, or consisting essentially of, or consisting of, administering an AAV vector expressing GAA as disclosed herein, in the absence of co-administration of enzyme replacement therapy (ERT) with GAA or other small molecule treatment for Pompe. In some embodiments, the method also comprises, or consists essentially of, or consists of, administering a AAV vector expressing GAA with minimal immune modulation, or in the absence of immune modulation.

In some embodiments, the method to treat Pompe Disease as disclosed herein comprises administering to the subject a pharmaceutical composition comprising, or consisting essentially of, or consisting of, an AAV vector expressing GAA as disclosed herein, where there administration is in the absence of concurrent treatment or administration of another therapy or treatment for Pompe, including but not limited to, enzyme replacement therapy (ERT) with GAA or other small molecule treatment for Pompe. That is, the methods disclosed herein relates to treatment of Pompe with administration to the subject a AAV expressing GAA, where there is no co-administration, or combination treatment, or concurrent treatment with GAA ERT.

In some embodiments, the subject has late onset Pompe Disease (LOPD) or infantile-onset Pompe disease.

The disclosure herein relates, in general, to a method to treat a subject with Pompe Disease, comprising, or consisting essentially of, administering to the subject a pharmaceutical composition comprising a recombinant adenovirus associated (AAV) vector comprising in its genome, a heterologous nucleic acid sequence encoding a polypeptide comprising an alpha-glucosidase (GAA) polypeptide, wherein the heterologous nucleic acid is operatively linked to a liver-specific promoter, and wherein the subject is not administered a GAA enzyme replacement therapy (ERT).

In all aspects disclosed herein, recombinant AAV (rAAV) vectors and constructs for rAAV for delivering a nucleic acid encoding GAA polypeptide to a subject for the treatment of Pompe Disease are disclosed are disclosed in International Patent Application WO 2020/102645 and WO2021102107A1 both of which are incorporated herein in their entirety by reference.

The inventors demonstrate herein that a rAAV vector, e.g., comprising a AAV8 capsid, a liver specific promoter (LSP) and the transgene for hGAA (herein referred to AAV8-LSPhGAA), when administered as a one-time single IV administration, selectively expresses and secretes GAA from transduced hepatocytes. The inventors demonstrate that the primary mechanism of action of the rAAV8-LSPhGAA secretes continuous low levels of GAA from the liver into the systemic circulation in order to provide therapeutic exposure levels of GAA to tissues, for example, in but not exclusively in the muscle, resulting in glycogen removal and restoration of cellular architecture and function. In addition, the inventors demonstrate that rAAV8-LSP-hGAA also mediates a regulatory T cell response in the host resulting in immunotolerance to the secreted GAA. Accordingly, the data presented in the Examples herein demonstrates that rAAV8-LSP-hGAA expressed GAA at a level that induce immune tolerance, and combats the potentially negative effects co-administration of ERT (e.g., alglucosidase alfa/LUMIZYME®), and thus suppresses inhibition of receptor-mediated uptake of GAA by neutralizing antibody.

In some embodiments, the recipient subject maintains substantially low T cell reactivity to the vector capsid. In some embodiments, the T cell reactivity is below a threshold range of >40 to <120 SFU/million PBMC such as measured by ELISpot reactivity. In some embodiments, the subject exhibits these levels by at least about 250 days after the AAV-LSP-hGAA administration and/or for at least about 80 days after ERT withdrawal.

In some embodiments, GAA expressed that comprises at least a signal peptide that promotes secretion of GAA polypeptide from the liver. In some embodiments, the GAA polypeptide, or modified GAA, is expressed as a fusion protein comprising at least a signal peptide that promotes secretion of the GAA polypeptide from the liver. In some embodiments, the GAA can be optionally fused to a targeting sequence that allows effective targeting to lysosomes in mammalian cells, e.g., muscle cells, for example, human cardiac and skeletal muscle cells.

In all aspects of all embodiments of the technology described herein, the liver specific promoter expresses the hGAA polypeptide preferentially in the liver. In all aspects of all embodiments of the technology described herein, the liver specific promoter expresses the hGAA polypeptide preferentially in the liver. In all aspects of all embodiments of the technology described herein, in some embodiments where the AAV vector comprises at least one capsid protein targeting the liver.

### I. Methods of Administration

The disclosure herein relates, in general, to a method to treat a subject with Pompe Disease, comprising administering to the subject with Pompe disease a pharmaceutical composition comprising, or consisting essentially of, a recombinant adenovirus associated (AAV) vector comprising in its genome, a heterologous nucleic acid sequence encoding a polypeptide comprising an alpha-glucosidase (GAA) polypeptide, wherein the heterologous nucleic acid is operatively linked to a liver-specific promoter, and wherein the subject is not administered a GAA enzyme replacement therapy (ERT) for an extended period of time, or can have extended breaks from ERT. In one embodiment, ERT is continued, but at least one of: dosage or frequency is reduced.

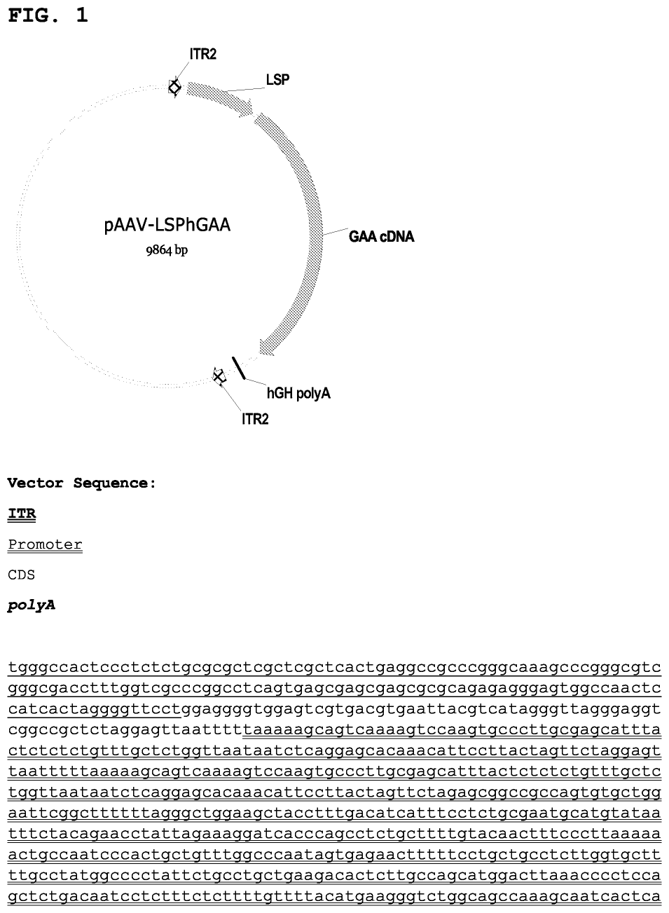

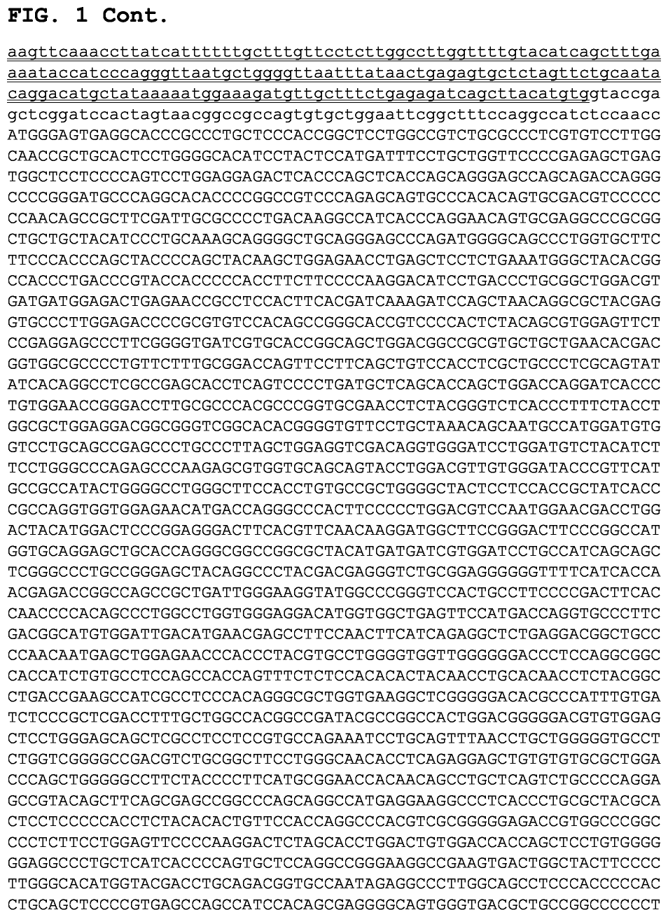

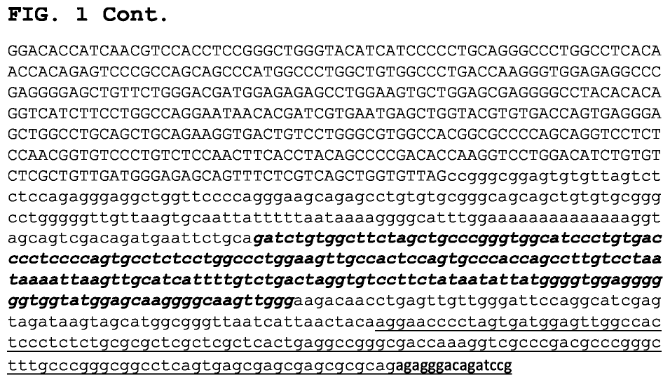

Disclosed herein in the Examples are the results of a human clinical trial for AAV-mediated gene transfer of GAA for Pompe disease. In particular, the Examples disclosed herein disclose the results of the clinical development program which was established to assess the therapeutic efficacy of administration of AAV8-LSPhGAA viral vector, which is a AAV8 expressing GAA under the control of a liver specific promoter (LSP) (see, e.g., FIG. 1 herein), and the ability to treat Pompe disease without concurrent ERT administration, and/or after ERT withdrawal.

In all aspects disclosed herein, a recombinant AAV (rAAV) vectors and constructs for rAAV for delivering a GAA polypeptide to a subject in the methods to treat Pompe Disease as disclosed herein are disclosed in International Patent Applications WO 2020/102645 and WO2021102107 both of which are incorporated herein in their entirety by reference.

Without wishing to be bound by theory, the inventors have discovered a method to treat Pompe disease with AAV-mediated delivery of GAA to the subject, where the rAAV expresses GAA to a steady state shortly after administration, such that the subject could be withdrawn from ERT as early as, or at, or around the time of the rAAV administration, e.g., ERT can be withdrawn the day before administration, the same day of administration, or the day after administration, or within a week, or within 2-weeks of administration. As disclosed herein, a steady state of GAA expression by the rAAV as disclosed herein is a serum level of GAA at a pharmacological activity range from about 160 to ≤2260 nmolmlhr, or about 189 to ≤2,260 nmol/mL/hr. In some embodiments, ERT is withdrawn when the subject demonstrates biochemical evidence of transgene GAA secretion. In some embodiments the subject also exhibits no clinically significant decline in motor function (6MWT) or function (FVC upright). In some embodiments ERT is withdrawn at anytime between about 24 weeks and about 104 weeks.

In some embodiments, the method to treat Pompe disease with rAAV expressing GAA as disclosed herein comprises administration of a therapeutically effective amount of a rAAV to result in a serum level of expressed hGAA within a pharmacological activity range of between 189 to 410 nmol/mL/hr, or 410 to ≤2,260 nmol/mL/hr.

In some embodiments, the method to treat Pompe disease with rAAV expressing GAA as disclosed herein comprises administration of a rAAV to result in a serum level of expressed hGAA within a range of 189 to ≤2,260 nmol/mL/hr, and where the subject achieves clinical stability of one or more symptoms of Pompe disease. Clinical stability includes a steady state in any one or more of the parameters: the 6MWT (6-minute walk test), FVC (Forced vital capacity). In some embodiments, clinical stability refers to a stable level in either motor function (as determined by the 6MWT) and/or pulmonary function (as determined by the FVC) in two consecutive assessments no less than 3-months apart. In some embodiments, a clinical stable level of motor function as determined by the 6MWT is ≤12% decline, or less than a 43-meter decrease from baseline in two consecutive assessments no less than 3-months apart. Stated differently, a clinical stable level of motor function as determined by the 6MWT position is within a 0-12% decline from a baseline level in two consecutive assessments no less than 3-months apart.

In some embodiments, a clinical stable level of pulmonary function as determined by the FVC % predicted in an upright position is ≤15% decrease from baseline in two consecutive assessments no less than 3-months apart. Stated differently, a clinical stable level of pulmonary function as determined by the % FVC predicted in an upright position is between 1-14% from a baseline in two consecutive assessments no less than 3-months apart.

In some embodiments, the baseline level of the 6MWT or FVC is the level measured at or before administration of the rAAV expressing GAA. In some embodiments, the baseline level of the 6MWT or FVC is the level measured at or before administration of the rAAV expressing GAA when the subject is concurrently administered GAA ERT. In some embodiments, the baseline level of the 6MWT or FVC is the level measured at or before administration of the rAAV expressing GAA when the subject is withdrawn from GAA ERT. In some embodiments, the baseline level of the 6MWT or, FVC is the level before withdrawing GAA ERT, e.g, at about 24 to about 26 weeks. In some embodiments, clinical stability is maintained between before ERT withdrawal and after ERT withdrawal of Pompe patients where the patients have received single administration of AAV comprising nucleic acid encoding GAA administrated at the time of ERT administration, before ERT administration, or, after ERT administration. Clinical stability is maintained indicate that 6MWT and or, FVC are within the ranges from baseline as described herein.

In some embodiments, the method to treat Pompe disease with rAAV expressing GAA as disclosed herein comprises administration of an amount of rAAV to result in a reduction of glycogen levels in one or more tissues to within a normal range, where the normal range is the glycogen levels in the comparative tissue of a subject without Pompe disease. In some embodiments of the invention, the glycogen content in tissue is reduced from baseline (e.g., measurements taken at or before day 1 of AAV-GAA administration) by about 10% to about 40%, about 10% to about 30%, or about 15% to about 25%.

### A. AAV-hGAA Dosages

As disclosed herein in the Examples, human subjects were administered a rAAV expressing GAA at a dose of 1.6E12 vg/kg (Cohort 1) with ERT withdrawal occurring at about 24 or 26 weeks after recombinant AAV administration—That is, the last dose of ERT for Cohort 1 subjects was administered at about 24 weeks after rAAV administration, and therefore the withdrawal of ERT occurs at, or around the beginning of week 26 after administration. In Cohort 1 there was therapeutic correction of disease pathophysiology even in the absence of ERT, and also protection against immune response to the expressed hGAA e.g, as measured by the antibodies against the expressed hGAA, therefore demonstrating that subjects with Pompe disease that are administered a AAV expressing hGAA as disclosed herein can have long term cessation of ERT.

In some embodiments the dose of the a rAAV vector or rAAV genome to be administered to the subject according to the method to treat Pompe Disease as disclosed herein depends upon the mode of administration, the severity of the Pompe disease or other condition to be treated and/or prevented, the individual subject's condition, the particular virus vector or capsid, the liver-specific promoter being used and the nucleic acid to be delivered, including but not limited to, nucleic acid encoding the signal peptide attached to the 5′ of the nucleic acid encoding expressible GAA polypeptide, and the like, and can be determined in a routine manner.

In some embodiments, the therapeutically effective amount of the rAAV vector expressing GAA is an amount that results in a serum GAA concentration at steady state similar to the GAA pharmacological activity achieved by long term GAA ERT (e.g within 5%, 10%, 20% of such levels). For example, a target GAA serum concentration at steady state ranging from about 160 to ≤2,260 nmol/mL/hr, from about 189 to ≤2,260 nmol/mL/hr, or ranging from 410 to ≤2,260 nmol/mL/hr. In some embodiments, the dose of the rAAV vector expressing GAA is a therapeutically effective amount to achieve a target GAA serum concentration at steady state that confers pharmacological activity ranges from 189 to ≤2,260 nmol/mL/hr. In some embodiments, the dose of the rAAV vector expressing GAA is a therapeutically effective amount to increase the tissue GAA levels in the subject to >0.30 μmol 4 MU/min/gram of tissue, where the normal range of tissue GAA content in a subject without Pompe disease is 0.36±/0.13 μmol 4 MU/min/gram of tissue. In some embodiments, the dose of the rAAV vector expressing GAA is a therapeutically effective amount to increase the tissue GAA levels in the subject to between 0.25 to 0.4 μmol 4 MU/min/gram of tissue. In some embodiments, the dose of the rAAV vector expressing GAA is a therapeutically effective amount to result in a normal tissue GAA content of about 0.36 μmol 4 MU/min/gram of tissue, e.g., about 0.25, or about 0.26, or about 0.27, or about 0.28, or about 0.29, or about 0.30, or about 0.31, or about 0.32, or about 0.33, or about 0.34, or about 0.35, or about 0.36, or about 0.37, or about 0.38, or about 0.39, or about 0.40 μmol 4 MU/min/gram of tissue.

In some embodiments, the dose of the rAAV vector expressing GAA is a therapeutically effective amount to increase the tissue GAA levels in the subject to between 0.1-0.5 μmol 4 MU/min/gram of tissue. In some embodiments, the dose of the rAAV vector expressing GAA is a therapeutically effective amount to result in a normal tissue GAA content of about greater than 0.36 mol 4 MU/min/gram of tissue. In some embodiments, the dose of the rAAV vector expressing GAA is a therapeutically effective amount to increase tissue GAA content or levels in the subject within the range 0.2-0.4 μmol 4 MU/min/gram of tissue. In some embodiments, the dose of the rAAV vector expressing GAA is a therapeutically effective amount to increase tissue GAA content in the subject to within 40%, or within 30%, or within 20%, or within 10%, or within 9%, 8%, 7%, 6%, 5%, 4%, 3%, 2% or 1% of the normal muscle tissue GAA content of 0.36±0.13 (mol 4 MU/min/gram of tissue), where the GAA content of normal muscle tissue is a reference level of GAA in a subject without Pompe Disease. In some embodiments, the dose of the rAAV vector expressing GAA is a therapeutically effective amount to increase tissue GAA content in the subject greater than 0.1 mol 4 MU/min/gram of tissue, where the normal range GAA content in subjects with Pompe disease is 0.05±0.04 (mol 4 MU/min/gram of tissue). In some embodiments, the dose of the rAAV vector expressing GAA is a therapeutically effective amount to increase tissue GAA content in the subject more than 2-fold, or 3-fold, or 4-fold, or 5-fold, or 6-fold, or 7-fold, or 8-fold, or 9-fold, or 10-fold, or more than 10-fold of the level of GAA tissue content in the subject with Pompe. In some embodiments, the dose of the rAAV vector expressing GAA is a therapeutically effective amount to increase tissue GAA content in the subject to about 50%, or, about 40%, or about 30%, or about 20%, or about 10%, or about 9%, about 8%, about 7%, about 6%, about 5%, about 4%, about 3%, about 2% or about 1% of the level of GAA tissue content in the subject with Pompe. In some embodiments, the GAA activity in muscle is at least 1.5 fold, at least 2 fold, at least 3 fold, at least 5 fold, at least 8 fold, or at least 10 fold than the level prior to AAV administration. In some embodiments, the GAA activity in muscle is at least 1.5 fold, at least 2 fold, at least 3 fold, at least 5 fold, at least 8 fold, or at least 10 fold than the level after the long term ERT was withdrawn for at least about 24 weeks.

In some embodiments, the dose of the rAAV vector expressing GAA is a therapeutically effective amount to reduce the tissue glycogen levels in the subject within the range 0.25% wet tissue weight to about 1.5% wet tissue weight. In some embodiments, the dose of the rAAV vector expressing GAA is a therapeutically effective amount to reduce the muscle tissue glycogen levels in the subject to within 40%, or within 30%, or within 20%, or within 10%, or within 9%, 8%, 7%, 6%, 5%, 4%, 3%, 2% or 1% of the normal muscle tissue glycogen content of 0.99%±0.74 (% wet tissue weight), which is the normal muscle tissue glycogen content (measured as % wet tissue weight), of a subject that does not have Pompe disease.

In some embodiments, the dose of the rAAV vector expressing GAA is a therapeutically effective amount of GAA to exhibit an improvement in the therapeutic index of 3- to 5-fold.

In some embodiments, the dose of the rAAV vector expressing GAA is a therapeutically effective amount to result in stability of one or more symptoms of Pompe disease such as determined by the clinical stability parameters as disclosed herein. Such parameters include a steady state in the 6MWT (6-minute walk test) and/or FVC (Forced vital capacity) in two consecutive assessments no less than 3-months apart as disclosed herein. In some embodiments, a clinical stable level of motor function as determined by the 6MWT is ≤12% decline, or less than a 43-meter decrease from baseline in two consecutive assessments no less than 3-months apart. In some embodiments, a clinical stable level of pulmonary function as determined by the FVC % predicted in an upright position is ≤15% decrease from baseline in two consecutive assessments no less than 3-months apart.

In some embodiments, the dose of the rAAV vector expressing GAA is a therapeutically effective amount to result in the subject having clinically stable levels of hGAA at 10-weeks, or at least 20 weeks, or 30 weeks post AAV administration.

In an embodiment, as used herein, without limitation, the term “effective amount” is synonymous with “therapeutically effective amount”, “effective dose”, or “therapeutically effective dose.” In an embodiment, the effectiveness of a therapeutic compound disclosed herein to treat Pompe Disease can be determined, without limitation, by observing an improvement in an individual based upon one or more clinical symptoms, and/or physiological indicators associated with Pompe Disease. In an embodiment, an improvement in the symptoms associated with Pompe Disease can be indicated by a reduced need for a concurrent therapy.

In some embodiments, where the rAAV vector expressing hGAA is AAV8-LSPhGAA as disclosed herein, exemplary doses for achieving therapeutic effects are titers of at least about 1.0E11 vg/kg, 1.1E11 vg/kg, 1.2E11 vg/kg, 1.3E11 vg/kg, 1.4E11 vg/kg, 1.5E11 vg/kg, 1.6E11 vg/kg, 1.7E11 vg/kg, 1.8E11 vg/kg, 1.9E11 vg/kg, about 1.0E12 vg/kg, about 1.1E12 vg/kg, about 1.2E12 vg/kg, about 1.3E12 vg/kg, about 1.4E12 vg/kg, about 1.5E12 vg/kg, about 1.6E12 vg/kg, about 1.7E12 vg/kg, about 1.8E12 vg/kg, about 1.9E12 vg/kg, about 2.0E12 vg/kg, about 3.0E12 vg/kg, about 4.0E12 vg/kg, about 5.0E12 vg/kg, about 6.0E12 vg/kg, about 7.0E12 vg/kg, about 8.0E12 vg/kg, about 9.0E12 vg/kg, about 1.0E13 vg/kg, about 1.2E13 vg/kg, about 1.2E13 vg/kg, about 1.2E13 vg/kg, about 1.3E13 vg/kg, about 1.4E13 vg/kg, about 1.5E13 vg/kg, about 1.6E13 vg/kg, about 1.7E13 vg/kg, about 1.8E13 vg/kg, about 1.9E13 vg/kg, about 2.0E13 vg/kg, about 3.0E13 vg/kg, about 4.0E13 vg/kg, about 5.0E13 vg/kg.

In preferred embodiments, exemplary doses for achieving therapeutic effects according to the methods as disclosed herein are titers of at between 1.2E11 and 4.0E11 vg/kg, for example, least about 1.0E11 vg/kg, 1.1E11 vg/kg, 1.2E11 vg/kg, 1.3E11 vg/kg, 1.4E11 vg/kg, 1.5E11 vg/kg, 1.6E11 vg/kg, 1.7E11 vg/kg, 1.8E11 vg/kg, 1.9E11 vg/kg. In other preferred embodiments, exemplary doses are titers of between 1.2E12 and 4.0E12 vg/kg, for example, least about 1.0E12 vg/kg, about 1.1E12 vg/kg, about 1.2E12 vg/kg, about 1.3E12 vg/kg, about 1.4E12 vg/kg, about 1.5E12 vg/kg, about 1.6E12 vg/kg, about 1.7E12 vg/kg, about 1.8E12 vg/kg, about 1.9E12 vg/kg, about 2.0E12 vg/kg, about 2.1E12 vg/kg, about 2.2E12 vg/kg, about 2.3E12 vg/kg, about 2.4E12 vg/kg, about 2.5E12 vg/kg, about 2.6E12 vg/kg, about 2.7E12 vg/kg, about 2.8E12 vg/kg, about 2.9E12 vg/kg, about 3.0E12 vg/kg, about 3.1E12 vg/kg, about 3.2E12 vg/kg, about 3.3E12 vg/kg, about 3.4E12 vg/kg, about 3.5E12 vg/kg, about 3.6E12 vg/kg, about 3.7E12 vg/kg, about 3.8E12 vg/kg, about 3.9E12 vg/kg, about 4.0E12 vg/kg.

In some embodiments, where the rAAV vector expressing hGAA is AAV8-LSPhGAA as disclosed herein, exemplary doses for achieving therapeutic effects are titers of at least about 1.0E11 to 4.0E11 vg/kg, or about 1.0E12 to 4.0E12 vg/kg, or about 1.2E12 to 3.0E12 vg/kg, or about 1.2E12 to 2.5E12 vg/kg, or about 2.5E12 to 4.0E12 vg/kg, or about 2.0E12 vg/kg to about 5E12 vg/kg or about 4.E12 vg/kg to about 2E13 vg/kg.

In some embodiments, the dosage may be modified by a person of ordinary skill in the art, e.g., the dose administered can be lower than 1.0E12 vg/kg, or lower than about 1.6E12 vg/kg where a stronger promoter than the LSP used in the AAV8-LSPhGAA vector disclosed herein is operatively linked to the nucleic acid encoding GAA. In contrast, in alternative embodiments, the dosage may be modified by a person of ordinary skill in the art, e.g., the dose of the rAAV vector administered can be higher than about 1.6E12 vg/kg, or higher than about 5.0E12 vg/kg when a weaker liver-specific promoter than the LSP used in the AAV8-LSPhGAA vector disclosed herein is operatively linked to the nucleic acid encoding GAA. Exemplary doses for achieving therapeutic effects are titers of at least about 1.0E⁵, 1.0E⁶, 1.0E⁷, 1.0E⁸, 1.0E⁹, 1.0E¹⁰, 1.0E¹¹, 1.0E¹² vg/kg, optionally about 1.0E¹⁰ to about 1.0E¹² transducing units (vg/kg), and optionally does not exceed about 4.0E¹² vg/kg or optionally is about 3.0E¹² transducing units (vg/kg).

Similarly, in some embodiments, the dosage may be modified by a person of ordinary skill in the art, e.g., the dose administered can be lower than 1.0E12 vg/kg, or lower than about 1.6E12 vg/kg where a stronger signal sequence than the endogenous GAA signal sequence used in the AAV8-LSPhGAA vector disclosed herein is linked to the 5′ of the expressed GAA polypeptide. In contrast, in alternative embodiments, the dosage may be modified by a person of ordinary skill in the art, e.g., the dose of the rAAV vector administered can be higher than about 1.6E12 vg/kg, or higher than about 1.6E13 vg/kg when an immune modulating agent is used.

In a further embodiment, administration of rAAV vector or rAAV genome according to the methods as disclosed herein to treat a subject with Pompe disease can result in production of a GAA protein with a circulatory half-life of 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 7 days, 1 week, 2 weeks, 3 weeks, 4 weeks, one month, two months, three months, four months or more.

In some embodiments, the methods for treatment of Pompe as disclosed herein relate to a single dose of a rAAV expressing hGAA is used to treat a subject in a single administration. However, in some embodiments, the dose of rAAV to be administered can be given to the subject in multiple administrations, e.g., a dose of rAAV can be divided into sub-doses and administered in multiple administrations.

In an embodiment, the time period of between administration of sub-doses of a rAAV vector according to the methods for treatment of Pompe as disclosed herein is selected from any of the following: 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

In some embodiments, it is envisioned that the methods for treatment of Pompe as disclosed herein can comprise multiple administrations of a single dose of a rAAV expressing hGAA, that is, the subject can be treated with a booster administration (i.e., a second, third, fourth, etc.) of a rAAV expressing hGAA after a defined period of time after the initial or first administration. The dose of a booster administration (i.e., 2^{nd}, 3^{rd}, 4^{th}, or 5^{th} etc.) can be the same dose (amount) of rAAV-hGAA administered in the first administration, or can be a higher dose, or a lower dose, depending on the factors above, including, but not limited to, a therapeutically effective dose to achieve any one or more of (i) serum GAA levels indicating steady state of GAA expression, (ii) reduced glycogen levels and/or, maintained glycogen levels within normal range in the muscle, and (iii) one or more Pompe symptoms, including muscle function and/or pulmonary function within clinically stable levels. As disclosed herein, a steady state of GAA expression by the rAAV as disclosed herein is a serum level of GAA at a pharmacological activity range from 165 to ≤2260 nmol/ml/hr or, from 189 to ≤2,260 nmol/mL/hr. Stability of one or more symptoms of Pompe disease can be determined by the clinical stability parameters as disclosed herein, and includes a steady state in the 6MWT (6-minute walk test) and/or FVC (Forced vital capacity) in two consecutive assessments no less than 3-months apart as disclosed herein. In some embodiments, a clinical stable level of motor function as determined by the 6MWT is ≤12% decline, or less than a 43-meter decrease from baseline in two consecutive assessments no less than 3-months apart. In some embodiments, a clinical stable level of pulmonary function as determined by the FVC % predicted in an upright position is ≤15% decrease from baseline in two consecutive assessments no less than 3-months apart.

In an embodiment, the time period of between administration of a first dose, and a subsequent dose (i.e., a booster dose) of a rAAV vector according to the methods for treatment of Pompe as disclosed herein is selected from any of the following: about 4 months, about 6 months, about 7 months, about 8 months, about 9 months, about 12 months, about 18 months, about 24 months, or about 3 years, about 4 years, about 5 years, or more than 5 years

In another embodiment, administration of a rAAV vector or rAAV genome as disclosed herein for the treatment of Pompe Disease results in an increase in weight by, e.g., at least 0.5 pounds, at least 1 pound, at least 1.5 pounds, at least 2 pounds, at least 2.5 pounds, at least 3 pounds, at least 3.5 pounds, at least 4 pounds, at least 4.5 pounds, at least 5 pounds, at least 5.5 pounds, at least 6 pounds, at least 6.5 pounds, at least 7 pounds, at least 7.5 pounds, at least 8 pounds, at least 8.5 pounds, at least 9 pounds, at least 9.5 pounds, at least 10 pounds, at least 10.5 pounds, at least 11 pounds, at least 11.5 pounds, at least 12 pounds, at least 12.5 pounds, at least 13 pounds, at least 13.5 pounds, at least 14 pounds, at least 14.5 pounds, at least 15 pounds, at least 20 pounds, at least 25 pounds, at least 30 pounds, at least 50 pounds.

In another embodiment, an AAV GAA of any serotype, as disclosed herein for the treatment of Pompe Disease results in an increase in weight by, e.g., from 0.5 pounds to 50 pounds, from 0.5 pounds to 30 pounds, from 0.5 pounds to 25 pounds, from 0.5 pounds to 20 pounds, from 0.5 pounds to 15 pounds, from 0.5 pounds to ten pounds, from 0.5 pounds to 7.5 pounds, from 0.5 pounds to 5 pounds, from 1 pound to 15 pounds, from 1 pound to 10 pounds, from 1 pound to 7.5 pounds, form 1 pound to 5 pounds, from 2 pounds to ten pounds, from 2 pounds to 7.5 pounds.

### B. Withdrawal of Long-Term ERT

Treatment of Pompe disease is normally by administration of long-term enzyme replacement therapy (ERT) with recombinant human acid α-glucosidase (rhGAA) and has previously reported to prolong survival of both LOPD and IOPD patients through improvement in pulmonary and muscle function. However, Schoser et al, report that after a period of stabilization, both these parameters continue to decline over time (see Schoser et al., 2017 Neurol, 264: 621-30). Additionally, ERT with recombinant GAA protein has numerous disadvantages, including but not limited to, short-half life of the administerted recombinant GAA in the blood, lack of efficient skeletal muscle updake, potential for high titer antibody response and even some patients failing to respond to ERT, and rigorous administration of a recombinant GAA infusion every 2 weeks, that can take between 5-8 hours. Moreover, while disease progression is slowed compared with untreated subjects, the benefits of ERT may not be long-lasting, and many patients die or remain weak despite treatment compliance (Tarnopolsky et al. 2016 Can J Neurol Sci, 43: 472-85).

The waning efficacy in subjects receiving recombinant GAA protein ERT is partially explained by an immune response to ERT where high, sustained anti-GAA antibody titers (HSAT) are formed. Subjects with HSAT demonstrated greatly increased mortality, in comparison with patients who formed no or only low titer antibodies (Banugaria et al. 2011). Furthermore, the ability to prevent antibody formation in subjects at risk for HSAT with immunosuppression, which significantly prolonged survival, confirmed the clinical relevance of HSAT (Mendelsohn et al. 2009; Banugaria et al. 2013; Kazi et al. 2017).

Subjects with Pompe's disease are considered as cross-reactive immunologic material (CRIM) positive if they have residual GAA enzyme activity and CRIM negative if no residual GAA activity is detected. Based on pooled clinical studies data, 28% of Pompe's disease cases are infantile-onset, of which about 85% are classic infantile-onset and three quarters of those are CRIM positive (Kemper, Comeau, and Green 2013). Determination of CRIM status in newly diagnosed IOPD patients is important since it allows for the institution of preventive immunomodulatory measures before beginning ERT, thus offsetting the worst damaging effects of HSAT. CRIM-negative Pompe disease subjects produced HSAT and demonstrated markedly reduced efficacy from ERT with rhGAA (Amalfitano et al. 2001). In the first pilot study of ERT in Pompe disease using Chinese hamster ovary cell-derived rhGAA, the two subjects who were CRIM-negative produced higher titers of anti-GAA antibodies than the third subject who was CRIM-positive. This corresponded with a markedly reduced efficacy of ERT in the CRIM-negative subjects. The relevance of antibody formation to efficacy of therapy in Pompe disease has been emphasized by the poor response of CRIM-negative subjects to ERT, which correlated with the onset of HSAT (Kishnani et al. 2010).

While no LOPD subjects are CRIM negative, some mount high antibody responses to rhGAA capable of interfering with optimal efficacy of ERT (Patel et al. 2012; de Vries et al. 2017; “LUMIZYME Prescribing Information” 2014). In addition, all Pompe subjects mount some level of anti-GAA antibody response with unknown effects on ERT efficacy.

As disclosed herein, the inventors have demonstrated herein that subjects with Pompe disease that are administered a AAV expressing hGAA as disclosed herein can have an extended period of cessation of the administration of long-term ERT. In particular, the Examples show data demonstrating that subject with Pompe disease who are administered a AAV expressing GAA polypeptide have the ability to reduce, or eliminate the clinical need for long-term hGAA ERT administration for an extended period of time.

Accordingly, the disclosed methods enable subjects with Pompe disease to withdraw from, or stop long-term administration of recombinant human GAA (rhGAA) ERT, which is normally administered on a weekly or every-other week regimen. In some embodiments, the methods disclosed herein enable a subject with Pompe disease to take breaks from the normal ERT regimen for extended period of time (e.g., extended periods of ERT cessation) if the subject is administered a specific dose of AAV vector expressing a GAA polypeptide as disclosed herein. In some embodiments, withdrawal of the administration of long-term ERT begins at about the time of administration of the AAV vector to the subject (e.g., the day before, the day of, or the day after), or in some embodiments, withdrawal of the administration of long-term ERT can occur at about 24 weeks, or anywhere within about 24 weeks to about 26 weeks after administration of the AAV vector. In some embodiments, the withdrawal of ERT begins anytime within a defined period (e.g., between a period of day 1 and 26 weeks, or day 1 and 6 months) after the administration of the AAV-GAA. In some embodiments, the withdrawal of ERT begins after the subject demonstrates biochemical evidence of transgene GAA secretion within a specified pharmacological activity range (e.g., discussed herein).

As disclosed herein, “long-term ERT” refer to the standard-of-care (SOC) treatment for a subject with Pompe disease, including IOPD and LOPD, and is normally a regimen of intravenous administration of recombinant human alglucosidase alfa protein (rhGAA) to the subject on a regular and frequent basis, e.g., every week or every 2 weeks, without any breaks in the regimen, and where the administered rhGAA protein provides an exogenous source of GAA. MYOZYME® (alglucosidase alfa) which was first US approved product (2006) for the treatment of Pompe disease and LUMIZYME® (alglucosidase alfa) which was approved in 2010 are exemplary current standard-of-care (SOC) treatments for infantile-onset and late-onset Pompe patients. The normal long-term ERT administration regimen is intravenously administration of Alglucosidase alfa every 2 weeks as an infusion at a dose of 20 mg/Kg (LUMIZYME Prescribing Information 2014).

As disclosed herein, the methods as disclosed herein enable the withdrawal or cessation of administration of long-term ERT for an extended period of time. In some embodiments, the extended period of time is at least about 3-months, or at least about 6-months, or at least about 1 year, or longer than 1 year.

As disclosed herein, “extended period” of time, as referred to in reference to time period that administration of long-term ERT is stopped, refers to a time period that is longer than 1 month, and in some embodiments is a time period longer than if up to 5 administrations of ERT are missed.

Accordingly, in some embodiments, the methods to treat a subject with Pompe Disease as disclosed herein comprises administering to the subject a pharmaceutical composition comprising a AAV expressing GAA and where the subject is not administered long-term GAA enzyme replacement therapy (ERT) for an extended period of time. In some embodiments, the cessation or withdrawal of administration of long-term ERT occurs anywhere between 1-2 days of administration and at least 24 weeks after the administration of the AAV-GAA vector. That is, in some embodiments, the subject being treated can stop the administration of ERT on the day of, or the day before or after administration of AAV-GAA. In some embodiments, the subject being treated according to the methods as disclosed herein can stop ERT after about 1 week, or about 2 weeks, or about 3 weeks, or about 1 month, or about 2 months, or about 3 months, or about 4 months, or about 5 months, or about 6 months after the administration of the AAV-GAA.

The exact timeframe for stopping ERT, or for ERT cessation, by each subject according to the methods as disclosed herein can be determined by an ordinary skilled practitioner, but without wishing to be limited by theory, encompassed herein is a method where ERT is stopped at time point that the serum GAA levels achieved from expression by the AAV-hGAA is near or about a serum level of within a pharmacological activity range of at least 165 nmol/ml/hr or, of at least 189 nmol/ml/hr, for example, between 189 to ≤2,260 nmol/mL/hr. In some embodiments, encompassed herein is a method where ERT is stopped at time point that the serum GAA levels achieved from expression by the AAV-hGAA is within 50%, or within 60%, or within 70% or within 80% of a serum level of within a pharmacological activity range of between 189 nmol/mL/hr. In some embodiments, encompassed herein is a method where ERT is stopped at time point that the serum GAA levels achieved from expression by the AAV-hGAA is within 50%, or within 60%, or within 70% or within 80% of a serum level of within a pharmacological activity range of between 165 to about 2000 nmol/mL/hr.

In some embodiments, encompassed herein is a method where ERT is stopped at time point that the serum GAA levels achieved from a normal ERT regimen are replaced with a GAA serum level achieved from expression by the AAV-hGAA. For example, as the serum GAA levels due to the recombinant hGAA from the last ERT administration declines, there is a concurrent increase in serum GAA levels achieved from expression by the AAV-hGAA, so that ERT withdrawal or cessation does not result in a decline in clinical stability of one or more symptoms of Pompe disease in the subject, as measured by the 6MWT or FVC according to the methods as disclosed herein. For illustration purposes only, in some embodiments, ERT withdrawal or cessation occurs when the administered AAV-hGAA results in the expressed GAA to achieve a serum GAA level for clinical stability of one or more symptoms of Pompe disease in the subject, for example, a clinical stable level of motor function as determined by the 6MWT is ≤12% decline, or less than a 43-meter decrease from baseline in two consecutive assessments no less than 3-months apart, or a clinical stable level of pulmonary function as determined by the FVC % in an upright position is ≤15% decrease from baseline in two consecutive assessments no less than 3-months apart, therefore making superfluous the recombinant hGAA from the last ERT administration.

In some embodiments, the methods as disclosed herein enables long term cessation of ERT for a period of about 1 year, or about 15 months, or about 18 months, or about 24 months, or about 30 months or more than 30 months (e.g., 3 years, 4 years, 5 years or more) while maintaining clinically stable with one or more symptoms of Pompe disease, such as measured by 6MWT and/or % FVC, GAA level in tissue or serum in the normal range, tissue glycogen levels within the normal range, or other symptoms as disclosed herein. For example, at least 73 weeks, at least 75 weeks, at least 80 weeks, at least 100 weeks, at least 150 weeks, at least 200 weeks and all points in between.

Accordingly, the methods as disclosed herein provide significant advantages to subjects with Pompe disease, including but not limited to reducing or eliminating the rigorous and arduous weekly, or every-other week infusions of long-term rhGAA ERT treatment, which are significantly time-consuming and geographically limiting, and hinders a patient with Pompe disease from travelling for prolonged periods from areas where their ERT infusions are administered. Additionally, as disclosed herein, the absence of ERT administration also reduces any side effects due to anti-rhGAA antibodies against the ERT, and also circumvents the need for administration of immune suppressants normally co-administered with the ERT. As such, the methods to treat Pompe disease as disclosed here leads to greater flexibility in Pompe treatment and an improvement in quality of life and lifestyle of subjects with Pompe disease.

### C. Administration of Complementary ERT

As disclosed herein, the methods to treat Pompe disease as disclosed herein by administering a AAV vector expressing GAA enables a subject to have breaks or “holidays” from the normal regimen of administration long-term ERT. That is, according to the methods as disclosed herein, a subject who is administered an AAV vector expressing GAA as disclosed herein can take extended periods of time in the absence of administration of long-term ERT. Moreover, in some embodiments, a subject administered a AAV vector expressing GAA as disclosed herein, can, after an initial period of withdrawal of the administration of long-term ERT for an extended period of time, be administered complementary ERT, where the complementary ERT is administered after about 6-months, or about 1 year, or longer than a year of cessation of the long-term ERT. As disclosed herein, the methods enable flexibility in normal long-term ERT administration regimens, allowing both extended breaks or absence of administration of long-term ERT which does not result in a clinical decline—that is, a subject remains clinically stable despite not having ongoing long-term ERT administration for an extended period of time.

In some embodiments, the methods as disclosed herein encompass re-administration of ERT (herein referred to as “complementary ERT”) after an extended period of time of cessation of ERT administration, and enable flexibility in normal ERT regimen, as the continued production of GAA expressed by the AAV permits include ERT flexibility. In some embodiments, the complementary ERT is pulse administration of ERT, as disclosed herein. In some embodiments, the complementary ERT is at less frequent intervals, or at a lower dose, or at irregular doses, or at irregular intervals as compared to the prior administration of long-term ERT.

In some embodiments, the methods as disclosed herein encompass recommencement of ERT (herein referred to as “complementary ERT”) after an extended period of at least 6 months to about 1 year of absence of long-term ERT administration. In some embodiments, complementary ERT can be for a short-period of time, and can be followed by a second extended period of ERT administration cessation. In some embodiments, complementary ERT can be for a period of anywhere between 3 months to about 2 years, for example, about 3 months, 4 months, 5 months, 6 months, 7 months, 8 months, 9 months, 10 months, 11 months, or for about 1 year.

In some embodiments, the method encompasses administering a rAAV expressing GAA according to as subject with Pompe, wherein administration of long-term ERT continues after administration of the recombinant AAV. However, the ERT is at a lower dose and/or frequency than before the administration of the recombinant AAV vector. For example, after administration of the AAV vector, long-term ERT can be administered every 3 weeks, once a month, bimonthly, once every 3 months, every 4 months, every 5 months, every 6 months for at least 24 weeks after administration of the AAV-GAA. Dosage of the long-term ERT can be reduced in one embodiment. In one method, a pulse administration regimen of long-term ERT after administration of the AAV vector can be used so that an irregular dosing schedule and/or amount can be used. As discussed herein, in some embodiments, administration of long-term ERT can be withdrawn at 24 weeks, or earlier as disclosed herein.

In some embodiments, due to continued expression of GAA by the administered AAV vector, the methods disclosed herein enable flexibility of administration of both long-term ERT or complementary ERT, such that if a subject plans to miss, or inadvertently or accidently misses one or more ERT administrations of a long-term ERT or complementary ERT regimen, the subject will maintain clinical stability. Currently, if ERT is missed, a much larger amount of ERT is needed to return to the same clinical level.

In some embodiments, the complementary ERT is at less frequent administration intervals, or at a lower dose, or at irregular doses, or at irregular administration intervals as compared to the prior administration of long-term ERT. For example, in some embodiments, the dose of rhGAA administered in a complementary ERT is less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 2%, or less than 1% of the normal dose of the rhGAA administered in a long-term ERT regimen.

In some embodiments, the complementary ERT is administered as pulse administration. In alternative embodiments, as a subject administered the rAAV vector compositions as disclosed herein can take breaks or interruptions from the regular dosing regimen of the long-term ERT administration or complementary ERT, where the long-term ERT or complementary ERT are administered by pulse administration. For example, in some embodiments, the administration of the long-term ERT or complementary ERT can be administered by pulsed administration. In certain embodiments, a subject administered the compositions can have pulsed administration of the long-term ERT or complementary ERT.

In certain embodiments, pulsed administration of the complementary ERT is suitable provided the subject has been administered the AAV vector composition as disclosed herein at a sufficient dose for continuous expression of GAA to maintain clinical stability and/or maintain a serum GAA level at or above 189 n mol/hr* (e.g., during the entire duration of the ERT break or “ERT holiday” where the regularly scheduled ERT is not administered). In certain embodiments, the methods disclosed herein allows a subject to undergo pulsed administration of complementary ERT for the lifetime of the subject.

In some embodiments, the regimen of administration of the complementary ERT can have intermittent breaks, where the administration of ERT is halted (e.g., the duration of the break or “ERT holiday” where the regimen of administration of ERT is halted).

In some embodiments, the methods encompass administration of complementary ERT by pulsed administration, where the pulsed administration of complementary ERT occurs least once a month, at least every other month, or at least every 6 months, or at least every year, or every other year. As the methods disclosed herein comprise administering a AAV vector at a sufficient dose for continuous expression of GAA in the subject and to maintain a serum GAA level at or above 189 n mol/hr*, if complementary ERT is administered, pulsed administration can substantially reduce the amount of ERT administered to the patient per dose or per total treatment regimen with an increased effectiveness, and allows for increased flexibility in a ERT administration regimen. This represents a significant saving in time, effort and expense and, more importantly, improved quality of life for Pompe patients, as well as a lower ERT dose which can lessens any side effects, including anti-GAA antibodies to the administered rhGAA protein.

In some embodiments, administration of complementary ERT is a pulsed administration. In certain embodiments, a pulsed administration comprises administering complementary ERT for about 8 weeks, followed by not administering complementary ERT for about 4 weeks. In some embodiments, the pulsed administration comprises administering complementary ERT for about 6 weeks (i.e., 6 weekly infusions, or 3 infusions every 2 weeks), followed by not administering a complementary ERT for about 2 weeks. In certain embodiments, the pulsed administration comprises administering complementary ERT for about 4 weeks, followed by not administering complementary ERT for about 2 weeks. In some embodiments, the pulsed administration comprises administering complementary ERT for about 2 weeks, followed by not administering complementary ERT for about 2 weeks.

In some embodiments, the pulsed administration of the complementary ERT is at less frequent intervals than conventional regimens for long-term ERT administration, e.g., the intervals for pulsed complementary ERT can be an administration or infusion of ERT about every 3 weeks, or about every 1 month, or about every 6 weeks, or about every 2 months, or about every 3 months, or about every 4 months. In some embodiments, the intervals between pulsed administrations of complementary ERT can be irregularly spaced, for example purposes only, administration of ERT can be spaced 3 weeks apart, then 4 weeks apart, then 6 weeks apart, then 3 weeks apart, etc., with the spacing determined based on clinical stability of the subject as disclosed herein, or simply convenience of timing and/or planning the complementary ERT administration.

In some embodiments, the interval between pulses can be determined by clinical stability parameters as disclosed herein. In alternative embodiments, the interval between pulses of administration of complementary ERT can be calculated by administering another dose of the ERT when the administered rhGAA is no longer detectable in the patient prior to delivery of the next pulse, or the serum GAA levels drop below a specific threshold, e.g., lower than a pharmaceutical activity range from 189 nmol/hr*ml. Intervals can also be calculated from the in vivo half-life of the administered rhGAA polypeptide in the MYOZYME® or LUMIZYME® formulations. Intervals may be calculated as greater than the in vivo half-life, or 2, 3, 4, 5 and even 10 times greater the composition half-life. For compositions with fairly rapid half-lives, intervals may be 25, 50, 100, 150, 200, 250 300 and even 500 times the half-life of rhGAA polypeptide. The number of pulses in administration of complementary ERT for an extended time period may be as little as two, but is typically from about 5 to 10, 10 to 20, 15 to 30 or more. In some embodiments, patients can receive administration of complementary ERT by pulse administration as disclosed herein for life according to the methods of this invention without the problems and inconveniences associated with current long-term ERT regimens.

### D. Absence of Long-Term Immune Suppression

Subjects administered rhGAA ERT are typically administered an immune suppressant with the ERT therapy, to avoid decreasing the ERT efficacy due to high, sustained anti-GAA antibody titers (HSAT) to the rhGAA. Importantly, it has previously been reported that the waning efficacy of ERT for the treatment of Pompe disease in subjects is partially explained by an immune response to ERT where high, sustained anti-GAA antibody titers (HSAT) are formed. Subjects with HSAT demonstrated greatly increased mortality, in comparison with patients who formed no or only low titer antibodies (Banugaria et al. 2011). Furthermore, the ability to prevent antibody formation in subjects at risk for HSAT with immunosuppression, which significantly prolonged survival, confirmed the clinical relevance of HSAT (Mendelsohn et al. 2009; Banugaria et al. 2013; Kazi et al. 2017).

Subjects with Pompe's disease are considered as cross-reactive immunologic material (CRIM) positive if they have residual GAA enzyme activity and CRIM negative if no residual GAA activity is detected. Based on pooled clinical studies data, 28% of Pompe's disease cases are infantile-onset, of which about 85% are classic infantile-onset and three quarters of those are CRIM positive (Kemper, Comeau, and Green 2013). Determination of CRIM status in newly diagnosed IOPD patients is important since it allows for the institution of preventive immunomodulatory measures before beginning ERT, thus offsetting the worst damaging effects of HSAT.

CRIM-negative Pompe disease subjects produced HSAT and demonstrated markedly reduced efficacy from ERT with rhGAA (Amalfitano et al. 2001). The relevance of antibody formation to efficacy of therapy in Pompe disease has been emphasized by the poor response of CRIM-negative subjects to ERT, which correlated with the onset of HSAT (Kishnani et al. 2010). While no LOPD subjects are CRIM negative, some mount high antibody responses to rhGAA capable of interfering with optimal efficacy of ERT (Patel et al. 2012; de Vries et al. 2017; “LUMIZYME Prescribing Information” 2014). In addition, all Pompe subjects mount some level of anti-GAA antibody response with unknown effects on ERT efficacy.

Herein, the inventors demonstrate that in Pompe disease subjects administered AAV-GAA, there were minimal anti-GAA antibodies generated to the GAA expressed by the AAV. Accordingly, in some embodiments, the methods disclosed herein encompass the administration with AAV-GAA to the subject without ongoing immune suppression. That is, in some embodiments, immune suppression is not administered to the subject long term.

In some embodiments, an immune suppressant or immune modulator (referred to interchangeably herein) is administered to the subject as an immune prophylaxis to the subject to prevent or reduce any immune response to the administered AAV vector, therefore allowing, if necessary, a subsequent or booster administration of the AAV vector expressing GAA according to the methods as disclosed herein.

In some embodiments, an immune modulator is administered for an initial period at, or around the time the AAV vector expressing GAA is administered to the subject. For example, an immune modulator is administered starting at about 24 hrs before AAV vector expressing GAA is administered to the subject. The initial administration period of the immune modulator (e.g., methotrexate/prednisone) can alternatively be 2 days before, 3 days before, 4, 5, 6 or 7 days before rAAV administration. In some embodiments, an immune modulator is administered starting at about 24 hrs before AAV administration and is administered for at least 1 day, or at least 2 days, or at least 3 days or at least 4 days, or at least 5 days, or at least 6 days, or for about 1 week, or for longer than 1 week after administration of the AAV vector expressing GAA. In some embodiments, an immune modulator is administered starting at, or about 24 hrs before AAV administration and is administered for no more than 1 day, or 2 days, 3 days, or 4 days, or 5 days, or 6 days, or for 1 week, or for 2 weeks, or for 3 weeks or for 1 month after administration of the AAV vector expressing GAA.

In some embodiments, an immune modulator is administered to the subject at tapering lower doses, e.g., at a first dose for a first period of time, at a second lower dose for a second period of time, and third dose that is lower than the second dose—for a third period of time, and so forth until no immune response to the AAV or GAA is produced. For example, in some embodiments, the first dose of an immune modulator is started at, or about 24 hrs before AAV administration and is administered for at least 1 day, or at least 2 days, or at least 3 days or at least 4 days, or at least 5 days, or at least 6 days, or for about 1 week, or about 2 weeks, or about 3 weeks, or about 4 weeks, after which the immune modulator is reduced to a second dose (which is lower than the first dose) for a second period of time (e.g., for at least 1 day, or at least 2 days, or at least 3 days or at least 4 days, or at least 5 days, or at least 6 days, or for about 1 week, or about 2 weeks, or about 3 weeks, or about 4 weeks), after which the immune modulator is reduced to a third dose (which is lower than the second dose) for a third period of time (e.g., for at least 1 day, or at least 2 days, or at least 3 days or at least 4 days, or at least 5 days, or at least 6 days, or for about 1 week).

For exemplary purposes only, in some embodiments, the methods to treat Pompe Disease as disclosed herein comprise administering prednisone as an immune suppressant, i.e., immune prophylaxis, at a first dose of 60 milligrams (given orally) starting 24 hours prior to AAV vector administration. In some embodiments, prednisone is continued at 60 mg/day po through the completion of week four after vector administration, after which, at the beginning of week 5 the prednisone dose is tapered to a second dose level of 55 mg/day po and maintained for 7 days. In some embodiments, at the Beginning of week 6 the dose is tapered to a third dose level of 50 mg/day po and maintained for 7 days etc., so that the dose of the immune suppressant (i.e., prednisone) is tapered on a weekly basis by 5 mg/day, after an initial immune suppressant dose for 4 weeks. In some embodiments, the first dose, e.g., prednisone. is less than 60 milligrams, (e.g. 55 milligrams, 50, 45, 40, 35, 30, 25, 20, 15, or 10 milligrams). In some embodiments another corticosteroid such as methyl prednisolone is used instead of prednisone, optionally in lower amounts. In some embodiments the dosage and regimen is sufficient to maintain T cell reactivity to vector capsid at substantially low levels.

In some embodiments prednisone is combined with another agent (e.g., methotrexate) as the immune suppressant. In some embodiments immune suppression is accomplished by administration of prednisone, methotrexate, sirolimus, tacrolimus, rapamycin or any combination thereof. Without being bound by theory, it is thought that combined treatment with prednisone and methotrexate suppresses CD4 and CD8 cell multiplication. The initial administration period of the second agent (e.g., methotrexate) need not be at the same time or follow the same course as the first agent (e.g., prednisone), and can be on the same day as the AAV administration (e.g, 1 hour before) or can alternatively be 1, 2, 3, 4, 5, 6 or 7 days before the AAV administration.

In some embodiments, prednisone is combined with methotrexate as the immune suppressant. For example, the prednisone is administered as described directly above (e.g., prednisone 60 mg once daily from 24 hours prior to AAV administration, for 4 weeks, then taper by 5 mg each week), and further methotrexate is administered (e.g., 1 hour prior to vector administration on study day 1). In some embodiments, the methotrexate is initially administered, for example at a total weekly dose of 30 mg for a period of time, which is then tapered. Typically, methotrexate is administered in a single day for a weekly dose. Folic acid can be administered (e.g., 1 mg/day) on non-methotrexate treatment days and held at that dose throughout the taper to end once the methotrexate ends. To maximize adsorption and bioavailability, single doses of methotrexate should not exceed 15 mg. Doses higher than 15 mg are given on a divided schedule (e.g., 30 mg is given as 15 mg 2× per day). In some embodiments, the initial methotrexate dose is continued for 12 weeks. After the initial dosing period, tapering is by reducing the weekly methotrexates dose by a specific amount every week (e.g., reduce by 5 mg each week). For example, in week 13, to achieve a total weekly dose of 25 mg methotrexate, the second daily dose of methotrexate is reduced by 5 mg such that 15 mg is given in the morning followed by 10 mg in the evening. In week 14, to achieve a total weekly dose of 20 mg methotrexate, the first daily dose of methotrexate is reduced by 5 mg such that 10 mg is given in the morning followed by 10 mg in the evening. In weeks 15, 16, and 17 methotrexate can be administered as a single dose. In week 15 to achieve a total weekly dose of 15 mg methotrexate, one 15 mg dose of methotrexate is administered. In week 16 to achieve a total weekly dose of 10 mg methotrexate, one 10 mg dose of methotrexate is administered. In week 17 to achieve a total weekly dose of 5 mg methotrexate, one 5 mg dose of methotrexate is administered. In week 18 methotrexate will no longer be administered.

Prednisone and methotrexate dose and/or administration regimen may be modified based on AST/ALT elevations as needed. In some embodiments the dosage and regimen is sufficient to maintain T cell reactivity to vector capsid at substantially low levels. In some embodiments the prednisone is administered as described directly above (prednisone 60 mg once daily from 24 hours prior to AAV administration, for 4 weeks, then taper by 5 mg each week), and further methotrexate is initially administered (e.g., 1 hour prior to vector administration on study day 1) in a weekly dose that is lower than 30 mg/week (e.g., 25 mg/week, 20, 15, 10, 7.5, or 5, mg/week) for a period of time, which can then be tapered off as appropriate (e.g., after 12 weeks). In some embodiments, to taper the methotrexate the dose is reduced by 2.5 mg/week after the initial period of time (e.g., 12 weeks) on the initial dosage. Folic acid can be administered (e.g. 1 mg/day) on non-methotrexate treatment days, and held at that dose throughout the taper to end once the methotrexate ends. In a preferred embodiment, the initial methotrexate dose is from 5 mg/week to 7.5 mg/week (e.g., 5, 5.5, 6, 6.5, 7, or 7.5 mg/week) for 12 weeks, and then tapered (e.g., by 2.5 mg/week). In some embodiments the methotrexate and folic acid are not tapered to 0, and instead low level doses are given for an extended period of time or indefinitely.

The use of prednisone is exemplified herein as an immune suppressant for immune prophylaxis according to the methods as disclosed herein. However, it is envisioned that prednisone can be readily substituted with a different immune modulator and administration regimen known by a person of ordinary skill in the art.

In some embodiments, normal immune prophylaxis for preventing immune reactivity to the expressed GAA is stopped, or withdrawn on day 1, or shortly before or after administration of the rAAV expressing GAA according to the methods as disclosed herein.

### (i) Immune Modulation and Immunosuppression:

As disclosed herein, in some embodiments, the methods disclosed herein encompass the administration with AAV-GAA to the subject without ongoing immune suppression. That is, in some embodiments, immune suppression is not administered to the subject long term, and is only administered for a short and pre-defined period, including an initial period (with an initial dose) and a tapering period (with incremental tapering doses) after the administration of the AAV vector expressing GAA to the subject. Accordingly, in some embodiments, the immune suppression is administered for between 4 weeks to up to about 15 weeks after the administration of the AAV vector expressing GAA to the subject, and can be administered in an initial and tapering doses as disclosed herein.

Accordingly, in some embodiments, the methods and compositions using the AAV vectors and AAV genomes as described herein, for treating Pompe, further comprises administering an immune modulator for an initial period followed by a tapering period. In some embodiments, the immune modulator can be administered at the time of rAAV vector administration, before rAAV vector administration or, after the rAAV vector administration.

In any embodiment of the methods and compositions as disclosed herein, a subject being administered a rAAV vector or rAAV genome as disclosed herein is also administered an immunosuppressive agent. Various methods are known to result in the immunosuppression of an immune response of a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference, is bortezomib. In another embodiment, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors 31 and 02, TNF and others that are publicly known).

In some embodiments, the immune modulator is an immunoglobulin degrading enzyme such as IdeS, IdeZ, IdeS/Z, Endo S, or, their functional variant. Non-limiting examples of references of such immunoglobulin degrading enzymes and their uses as described in U.S. Pat. Nos. 7,666,582, 8,133,483, US 20180037962, US 20180023070, US 20170209550, U.S. Pat. No. 8,889,128, WO2010/057626, U.S. Pat. Nos. 9,707,279, 8,323,908, US 20190345533, US 20190262434, and WO2020/016318, each of which are incorporated in their entirety by reference.

In some embodiments, the immune modulator or immunosuppressive agent is a proteasome inhibitor. In certain aspects, the proteasome inhibitor is Bortezomib. In some aspects of the embodiment, the immune modulator comprises bortezomib and anti CD20 antibody, Rituximab. In other aspects of the embodiment, the immune modulator comprises bortezomib, Rituximab, methotrexate, and intravenous gamma globulin. Non-limiting examples of such references, disclosing proteasome inhibitors and their combination with Rituximab, methotrexate and intravenous gamma globulin, as described in U.S. Pat. Nos. 10,028,993, 9,592,247, and, 8,809,282, each of which are incorporated in their entirety by reference. One such proteasome inhibitor known in the art, for instance as disclosed in U.S. Pat. No. 9,169,492 and U.S. patent application Ser. No. 15/796,137, both of which are incorporated herein by reference, is bortezomib.

In another embodiment, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors β1 and β2, TNF and others that are publicly known).

In alternative embodiments, the immune modulator is an inhibitor of the NF-kB pathway. In certain aspects of the embodiment, the immune modulator is Rapamycin or, a functional variant. Non-limiting examples of references disclosing rapamycin and its use described in U.S. Pat. No. 10,071,114, US 20160067228, US 20160074531, US 20160074532, US 20190076458, U.S. Pat. No. 10,046,064, are incorporated in their entirety. In other aspects of the embodiment, the immune modulator is synthetic nanocarriers comprising an immunosuppressant. Non limiting examples of references of immunosuppressants, immunosuppressants coupled to synthetic nanocarriers, synthetic nanocarriers comprising rapamycin, and/or, tolerogenic synthetic nanocarriers, their doses, administration and use as described in US20150320728, US 20180193482, US 20190142974, US 20150328333, US20160243253, U.S. Pat. No. 10,039,822, US 20190076522, US 20160022650, U.S. Pat. Nos. 10,441,651, 10,420,835, US 20150320870, US 2014035636, U.S. Pat. Nos. 10,434,088, 10,335,395, US 20200069659, U.S. Pat. No. 10,357,483, US 20140335186, U.S. Pat. Nos. 10,668,053, 10,357,482, US 20160128986, US 20160128987, US 20200038462, US 20200038463, each of which are incorporated in their entirety by reference.

In some embodiments, the immune modulator is synthetic nanocarriers comprising rapamycin (ImmTOR™ nanoparticles) (Kishimoto, et al., 2016, Nat Nanotechnol, 11(10): 890-899; Maldonado, et al., 2015, PNAS, 112(2): E156-165), as disclosed in US20200038463, U.S. Pat. No. 9,006,254 each of which is incorporated herein in its entirety. In some embodiments, the immune modulator is an engineered cell, e.g., an immune cell that has been modified using SQZ technology as disclosed in WO2017192786, which is incorporated herein in its entirety by reference.

In some embodiments, the immune modulator is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In another further embodiment, the immunomodulator or adjuvant is poly-ICLC.

In some embodiments, the immune modulator is a small molecule that inhibit the innate immune response in cells, such as chloroquine (a TLR signaling inhibitor) and 2-aminopurine (a PKR inhibitor), can also be administered in combination with the composition comprising at least one rAAV as disclosed herein. Some non-limiting examples of commercially available TLR-signaling inhibitors include BX795, chloroquine, CLI-095, OxPAPC, polymyxin B, and rapamycin (all available for purchase from INVIVOGEN™). In addition, inhibitors of pattern recognition receptors (PRR) (which are involved in innate immunity signaling) such as 2-aminopurine, BX795, chloroquine, and H-89, can also be used in the compositions and methods comprising at least one rAAV vector as disclosed herein for in vivo protein expression as disclosed herein.

In some embodiments, a rAAV vector can also encode a negative regulators of innate immunity such as NLRX1. Accordingly, in some embodiments, a rAAV vector can also optionally encode one or more, or any combination of NLRX1, NS1, NS3/4A, or A46R. Additionally, in some embodiments, a composition comprising at least one rAAV vector as disclosed herein can also comprise a synthetic, modified-RNA encoding inhibitors of the innate immune system to avoid the innate immune response generated by the tissue or the subject.

In some embodiments, an immune modulator for use in the administration methods as disclosed herein is an immunosuppressive agent. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell costimulation pathways, such as agents that interfere with the coupling of T-cells and B-cells via the CTLA4 and B7 pathways, as disclosed in U.S. Patent Pub. No 2002/0182211. In one embodiment, an immunosuppressive agent is cyclosporine A. Other examples include myophenylate mofetil, rapamicin, and anti-thymocyte globulin. In one embodiment, the immunosuppressive drug is administered in a composition comprising at least one rAAV vector as disclosed herein, or can be administered in a separate composition but simultaneously with, or before or after administration of a composition comprising at least one rAAV vector according to the methods of administration as disclosed herein. An immunosuppressive drug is administered in a formulation which is compatible with the route of administration and is administered to a subject at a dosage sufficient to achieve the desired therapeutic effect. In some embodiments, the immunosuppressive drug is administered transiently for a sufficient time to induce tolerance to the rAAV vector as disclosed herein.

Various methods are known to result in the immunosuppression of an immune response of a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference, is bortezomib. In some embodiments, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors 31 and 02, TNF and others that are publicly known).

The use of such immune modulating agents facilitates the ability to for one to use multiple dosing (e.g., multiple administration) over numerous months and/or years. This permits using multiple agents as discussed below, e.g., a rAAV vector encoding multiple genes, or multiple administrations to the subject.

### E. Pharmaceutical Compositions

The rAAV vectors as disclosed herein (e.g., rAAV vector expressing GAA), such as for use in the methods of administration as disclosed herein can be formulated in a pharmaceutical composition with a pharmaceutically acceptable excipient, i.e., one or more pharmaceutically acceptable carrier substances and/or additives, e.g., buffers, carriers, excipients, stabilizers, etc. The pharmaceutical composition may be provided in the form of a kit. Pharmaceutical compositions comprising the rAAV vectors as disclosed herein for use in the methods of administration as disclosed herein and uses thereof are known in the art.

Accordingly, a further aspect of the invention provides a pharmaceutical composition comprising a rAAV vector as disclosed herein for use in the methods of administration as disclosed herein. Relative amounts of the active ingredient (e.g. a rAAV vectors aa disclosed herein), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1 percent and 99 percent (w/w) of the active ingredient. By way of example, the composition may comprise between 0.1 percent and 100 percent, e.g., between 0.5 and 50 percent, between 1-30 percent, between 5-80 percent, at least 80 percent (w/w) active ingredient.

The pharmaceutical compositions can be formulated using one or more excipients or diluents to (1) increase stability; (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload of the invention. In some embodiments, a pharmaceutically acceptable excipient may be at least 95 percent, at least 96 percent, at least 97 percent, at least 98 percent, at least 99 percent, or 100 percent pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia. Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21 st Edition, A. R. Gennaro, Lippincott, Williams and Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

The rAAV vectors as disclosed herein for use in the methods of administration as disclosed herein may be used in combination with one or more other therapeutic, prophylactic, research or diagnostic agents. By “in combination with,” it is not intended to imply that the agents must be administered at the same time and/or formulated for delivery together, although these methods of delivery are within the scope of the present invention. Compositions can be administered concurrently with, prior to, or subsequent to, one or more other desired therapeutics or medical procedures. In some embodiments, the delivery of one treatment (e.g., gene therapy vectors) is still occurring when the delivery of the second (e.g., one or more therapeutic) begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The composition described herein and the at least one additional therapy can be administered simultaneously, in the same or in separate compositions, or sequentially. For sequential administration, the gene therapy vectors described herein can be administered first, and the one or more therapeutic can be administered second, or the order of administration can be reversed. The gene therapy vectors and the one or more therapeutic can be administered during periods of active disorder, or during a period of remission or less active disease. The gene therapy vectors can be administered before another treatment, concurrently with the treatment, post-treatment, or during remission of the disorder.

When administered in combination, the rAAV vectors as disclosed herein for use in the methods of administration as disclosed herein and the one or more therapeutic (e.g., second or third therapeutic), or all, can be administered in an amount or dose that is higher, lower or the same as the amount or dosage of each used individually, e.g., as a monotherapy. In certain embodiments, the administered amount or dosage of a rAAV vector as disclosed herein for use in the methods of administration as disclosed herein and the one or more therapeutic (e.g., second or third agent), or all, is lower (e.g., at least 20%, at least 30%, at least 40%, or at least 50%) than the amount or dosage of each used individually. In other embodiments, the amount or dosage of the rAAV vector as disclosed herein for use in the methods of administration as disclosed herein and the one or more therapeutic (e.g., second or third agent), or all, that results in a desired effect (e.g., treatment of a cardiovascular disease or heart disease) is lower (e.g., at least 20%, at least 30%, at least 40%, or at least 50% lower) than the amount or dosage of each individually required to achieve the same therapeutic effect.

In some embodiments, the methods of administration of a rAAV vector as disclosed herein can deliver a rAVV vector disclosed herein alone, or in combination with an additional agent, for example, an immune modulator as disclosed herein.

In some embodiments, the AAV vectors expressing GAA as disclosed herein are not administered concurrently with, or in combination with ERT. In alternative embodiments, the AAV vectors expressing GAA as disclosed herein are administered in combination with ERT for a maximum period of 24 weeks or shorter than 24 weeks after administration of the AAV expressing ERT. In some embodiments, the AAV vectors expressing GAA as disclosed herein are administered in combination with an immune modulator for an initial period and, optionally a tapering period after administration of the AAV expressing ERT.

Accordingly, in one aspect, provided herein is a composition (e.g., a pharmaceutical composition) comprising the recombinant AAV vector particles described herein. Generally, the composition comprises the recombinant AAV vector particles described herein at a concentration from about 1e⁹ vg/ml to about 1e⁵ vg/ml. In some embodiments, the composition comprises the recombinant AAV vector particles described herein at a concentration from about 1e¹⁰ vg/ml to about 1e¹⁴ vg/ml. In some embodiments, the composition comprises the recombinant AAV vector particles described herein at a concentration from about 1e¹² vg/ml to about 1e¹⁴ vg/ml. In some embodiments, the composition comprises the recombinant AAV vector particles described herein at a concentration from about 1e¹² vg/ml to about 1e¹⁵ vg/ml. For example, the composition comprises the recombinant AAV vector particles described herein at a concentration from about 3e¹² vg/ml to about 3e¹³ vg/ml, from about 2.5e¹² vg/ml to about 1e¹⁴ vg/ml, from about 3e¹³ vg/ml to about 1e¹⁴ vg/ml, or from 1e¹³ vg/ml to about 1e¹⁴ vg/ml.

In some embodiments, the composition comprises the recombinant AAV vector particles described herein at a concentration of about 1e¹² vg/ml, or about 1.5e¹² vg/ml, or about 2e¹² vg/ml, or about 2.5e¹² vg/ml, or about 3e¹² vg/ml, or about 3.5e¹² vg/ml, or about 4e¹² vg/ml, or about 4.5e¹² vg/ml, or about 5e¹² vg/ml, or about 5.5e¹² vg/ml, or about 6e¹² vg/ml, or about 6.5e¹² vg/ml, or about 7e¹² vg/ml, or about 7.5e¹² vg/ml, or about 8e¹² vg/ml, or about 8.5e¹² vg/ml, or about 9e¹² vg/ml, or about 9.5e¹³ vg/ml, or about 1e¹³ vg/ml, or about 1.5e¹³ vg/ml, or about 2e¹³ vg/ml, or about 2.5e¹³ vg/ml, or about 3e¹³ vg/ml, or about 3.5e¹³ vg/ml, or about 4e¹³ vg/ml, or about 4.5e¹³ vg/ml, or about 5e¹³ vg/ml, or about 5.5e¹³ vg/ml, or about 6e¹³ vg/ml, or about 6.5e¹³ vg/ml, or about 7e¹³ vg/ml, or about 7.5e¹³ vg/ml, or about 8e¹³ vg/ml, or about 8.5e¹³ vg/ml, or about 9e¹³ vg/ml, or about 9.5e¹³ vg/ml, or about 1e¹⁴ vg/ml.

In some embodiments, the composition comprises a buffer. It is noted that any physiological buffer can be used. Non-limiting examples of buffers include, but are not limited to, PBS, Tris·HCl, phosphate, citric acid, histidine, tromethamine, succinic acid, malic acid, α-ketoglutaric acid, carbonate (bicarbonate-carbonic acid buffer), and protein buffers. In some embodiments, the buffer is PBS. In some embodiments, the buffer comprises Tris. In some embodiments, buffer is Tris·HCl. In some embodiments, the buffer is histidine buffer.

Generally, the buffer has a salt concentration of from about 50 mM to about 750 mM. For example, the buffer has a salt concentration from about 75 mM to about 700 mM, from about 100 mM to about 650 mM, from about 120 mM to about 600 mM, or from about 140 mM to about 550 mM. In some embodiments, the buffer has a salt concentration from about 150 mM to about 400 mM. In some embodiments, the buffer has a salt concentration of about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, or about 475 mM. In some preferred embodiments, the buffer has a salt concentration of about 150 mM, about 200 mM or about 365 mM.

In some embodiments, the ionic strength of the composition is at least about 100 mM. For example, the ionic strength of the composition is from about 125 mM to about 750 mM, or from about 150 mM to about 500 mM, or from about 175 mM to about 700 mM, from about 200 mM to about 600 mM, or from about 225 mM to about 550 mM, or from about 250 mM to about 500 mM, or from about 275 mM to about 450 mM, or from about 300 mM to about 400 mM. In some embodiments, the ionic strength of the composition is at least about 125 mM, at least about 150 mM, at least about 175 mM, at least about 200 mM, at least about 225 mM, at least about 250 mM, at least about 275 mM, at least about 300 mM, at least about 325 mM, at least about 350 mM, at least about 375 mM, at least about 400 mM, at least about 425 mM, at least about 450 mM, at least about 475 mM or at least about 500 mM. In some embodiments, the ionic strength of the composition is less than 100 mM, for example about 95 mM, about 90 mM, about 85 mM, about 80 mM, about 75 mM, about 70 mM, about 65 mM, about 60 mM, about 55 mM, about 50 mM, or, even less.

The osmolarity of the composition is maintained at near isotonic levels. For example, the osmolarity of the composition can be from about 100 mOsm to about 600 mOsm, such as from about 125 mOsm to about 500 mOsm, or, from about 130 mOsm to about 350 mOsm, or, from about 140 mOsm to about 400 mOsm, or, from about 140 mOsm to about 350 mOsm, or from about 200 mOsm to about 400 mOsm, or from about 500 mOsm to about 600 mOsm, or from about 200 mOsm to about 600 mOsm, or from about 300 mOsm to about 600 mOsm, or from about 200 mOsm to about 500 mOsm, or from about 300 mOsm to about 400 mOsm, or from about 150 mOsm to about 350 mOsm, or from about 175 mOsm to about 300 mOsm, or from about 300 mOsm to about 375 mOsm, or from about 200 mOsm to about 350 mOsm, or from about 225 mOsm to about 325 mOs, or from about 525 mOsm to about 590 mOsm. In some embodiments, the composition comprises an isotonic solution.

Generally, the composition has a pH of about 6.5 to about 8.0. For example, the composition has a pH of about 6.5 to about 7.5. In some embodiments, the composition has a pH of from about 7 to about 8. For example, the composition has a pH of from about 7.3 to about 7.9. In some other non-limiting example, the composition has a pH of from about 7.4 to about 7.8 or from about 7.4 to about 7.7. In some embodiments, the composition has a pH of from about 7.3 to about 7.6, e.g., from about 7.3 to about 7.55. In some preferred embodiments, the composition has a pH less than about 7.5. For example, the composition has a pH about 7.4 or lower, about 7.3 or lower, about 7.2 or lower, about 7.1 or lower, about 7.0 or lower, about 6.9 or lower, about 6.8 or lower, about 6.7 or lower, about 6.6 or lower, or about 6.5 or lower.

The composition can comprise one or more ions and/or salts thereof. Exemplary ions include, but are not limited to sodium, potassium, chloride, magnesium ammonium, carbonate, nitrate, chlorate, chlorite, and calcium. The ions can be provided as a salt, such as a halide (F, Cl, Br, I) salt of sodium, potassium, magnesium, and/or calcium, non-limiting examples of which include NaCl, KCl, MgCl₂, CaCl₂, and combinations thereof. Additional exemplary salts that can be used include, but are not limited to, carboxylic acid salts, such as acetates, propionates, pyrrol idonecarboxylates (or pidolates) or sorbates; poly hydroxylated carboxylic acid salts, such as gluconates, heptagluconates, ketogluconates, lactate gluconates, ascorbates or pantothenates; mono- or polycarboxyl hydroxy acid salts, such as citrates or lactates; amino acid salts, such as aspartates or glutamates; and fulvate salts. The salts are individually included at a concentration of from about 500 M to about 500 mM.

In some embodiments, the composition comprises one or more multivalent ions and/or salts thereof. Exemplary multivalent ions include, but are not limited to, calcium, citrate, sulfate, magnesium, and phosphate. Multivalent ions and/or salts thereof can be individually included in the composition at a concentration of from about 500 M to about 500 mM, for example, at a concentration of about 500 M, about 750 M, about 1 mM, about 1.3 mM, about 1.5 mM, about 1.7 mM, about 2.3 mM, about 2.5 mM, about 2.7 mM, about 3.3 mM, about 3.5 mM, about 3.7 mM, about 4.3 mM, about 4.5 mM, about 4.7 mM, about 5 mM, about 10 mM, about 25 mM, about 50 mM, about 75 mM, about 80 mM, about 85 mM, about 90 mM, about 95 mM, about 100 mM, about 125 mM, about 150 mM, about 175 mM, about 200 mM, about 225 mM, about 250 mM, about 275 mM, about 300 mM, about 325 mM, about 350 mM, about 375 mM, about 400 mM, about 425 mM, about 450 mM, about 475 mM, or about 500 mM. Non limiting examples of salts are NaCl, KCl, CaCl₂, CaSO₄, MgSO₄, Na₃PO₄, CaCO₃, NaNO₃, Al₂(SO₄)₃.

In some embodiments, the composition comprises NaCl. When present, NaCl can be at a concentration from about 100 mM to about 500 mM, or from about 125 mM to about 450 mM, or from about 100 mM to about 200 mM, or from about 150 mM to about 200 mM. For example, the composition can comprise NaCl at a concentration from about 150 mM to about 425 mM, from about 175 mM to about 400 mM, or from about 175 mM to about 375 mM, or from about 200 mM to about 375 mM.

In some embodiments, the composition comprises KCl. When present, KCl can be at a concentration from about 1 mM to about 10 mM. For example, the composition can comprise KCl at a concentration from about 1.5 mM to about 7.5 mM.

In some embodiments, the composition comprises CaCl₂. When present, CaCl₂ can be at a concentration from about 0.1 mM to about 2 mM. For example, the composition can comprise CaCl₂ at a concentration from about 0.5 mM to about 1.5 mM. In some embodiments, the composition comprises CaCl₂ at a concentration from about 0.75 mM to about 1.25 mM.

In some embodiments, the composition comprises MgCl₂. When present, MgCl₂ can be at a concentration from about 0.1 mM to about 1.5 mM. For example, the composition can comprise MgCl₂ at a concentration from about 0.25 mM to about 1 mM or from about 0.25 mM to about 0.75 mM.

In some embodiments, the composition comprises MgSO₄. When present, MgSO₄ can be at a concentration from about 5 mM to about 150 mM. For example, the composition can comprise MgSO₄ at a concentration from about 10 mM to about 120 mM, or from about 10 mM to about 50 mM, or from about 15 mM to about 45 mM, or about 75 mM to about 125 mM, or from about 80 mM to about 100 mM, or from about 85 mM to about 95 mM, or from about 15 mM to about 100 mM.

In some embodiments, the composition comprises phosphate, e.g., mono basic or dibasic phosphate or a salt thereof. When present, the phosphate, e.g., mono basic or dibasic phosphate or a salt thereof can be at a concentration from about 5 mM to about 30 mM. For example, the composition can comprise phosphate, e.g., mono basic or dibasic phosphate or a salt thereof at a concentration from about 7.5 mM to about 25 mM. In some embodiments, the composition comprises phosphate, e.g., mono basic or dibasic phosphate or a salt thereof at a concentration from about 10 mM to about 20 mM.

In some embodiments, the composition comprises a mono basic phosphate or a salt thereof at a concentration from about 0.25 mM to about 3 mM. For example, the composition comprises a mono basic phosphate or a salt thereof at a concentration from about 0.5 mM to about 2.75 mM, or from about 0.75 mM to about 2.5 mM or from about 1 mM to about 2.25 mM. In some embodiments, the mono basic phosphate or salt thereof is potassium phosphate monobasic.

In some embodiments, the composition comprises a dibasic phosphate or a salt thereof at a concentration from about 5 mM to about 15 mM. For example, the composition comprises a dibasic phosphate or a salt thereof at a concentration from about 7.5 mM to about 12.5 mM or from about 8 mM to about 10 mM. In some embodiments, the dibasic phosphate or a salt thereof is sodium phosphate dibasic. In some embodiments, the composition is substantially free of dibasic phosphate, e.g., sodium phosphate dibasic.

In some embodiments, the composition comprises Tris (e.g., Tris·HCl) or a salt thereof at a concentration from about 1 mM to about 50 mM. For example, the composition comprises Tris (e.g., Tris·HCl) or a salt thereof at a concentration of from about 5 mM to about 40 mM, or from about 7.5 mM to about 35 mM, or from about 10 mM to about 30 mM or from about 15 mM to about 25 mM.

In some embodiments, the composition comprises histidine or a salt thereof at a concentration from about 1 mM to about 50 mM. For example, the composition comprises histidine or a salt thereof at a concentration of from about 5 mM to about 40 mM, or from about 7.5 mM to about 35 mM, or from about 10 mM to about 30 mM or from about 15 mM to about 25 mM.

The pharmaceutical composition can also comprise a bulking agent. Exemplary bulking agents include, but are not limited to sugars, polyols and (PVP K24). Exemplary polyols include, but are not limited to, polyhydroxy hydrocarbons, monosaccharides, disaccharides, and trisaccharides. Some exemplary polyols include but are not limited to, sorbitol, mannitol, glycerol, propylene glycol, polyethylene glycol, dulcitol, sucrose, lactose, maltose, trehalose and dextran. In some embodiments, polyol is sorbitol, sucrose or mannitol. In some embodiments, the bulking agent is sorbitol. In some embodiments, the bulking agent is sucrose. In some embodiments, the bulking agent is mannitol. In some embodiments, the bulking agent is trehalose, e.g., trehalose dehydrate. In some embodiments, the bulking agent is a dextran, e.g., Dextran T40 and/or Dextran T10.

When present, the bulking agent can be present at a concentration of from about 0.5% (w/v) to about 10% (w/v). For example, the composition can comprise a bulking agent, e.g., a polyol or providone (PVP K24) at a concentration from about from about 1% (w/v) to about 7.5% (w/v), e.g., from about 1% (w/v) to about 4% (w/v) or from about 4% (w/v) to about 6% (w/v).

In some embodiments, the composition comprises glycerol, sorbitol, sucrose, or mannitol at a concentration from about 1% (w/v) to about 10% (w/v). In some embodiments, the composition comprises glycerol, sorbitol, sucrose, or mannitol at a concentration from about 1% (w/v) to about 10% (w/v). In some embodiments, the composition comprises sorbitol at concentration from about 3% (w/v) to about 6% (w/v). In some embodiments, the composition comprises sorbitol at concentration of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In some embodiments, the composition comprises sucrose at concentration from about 3% (w/v) to about 6% (w/v). In some embodiments, the composition comprises sucrose at concentration of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v). In some embodiments, the composition comprises mannitol at concentration from about 3% (w/v) to about 6% (w/v). In some embodiments, the composition comprises mannitol at concentration of about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v).

The composition can also comprise a non-ionic surfactant. The non-ionic surfactant can be selected from the group consisting of polyoxyethylene fatty alcohol ethers, polyoxyethylene alkyl phenyl ethers, polyoxyethylene-polyoxypropylene block copolymers, alkylglucosides, alkyl phenol ethoxylates, preferably polysorbates, polyoxyethylene alkyl phenyl ethers, and any combinations thereof. Non-limiting examples of suitable non-ionic surfactants include polyoxyethylene (12) isooctylphenyl ether (e.g., IGEPAL® CA-270 polyoxyethylene (12) isooctylphenyl ether), polyoxyethylenesorbitan monooleate (e.g., TWEEN® 80 polyoxyethylenesorbitan monooleate), polyethylene glycol octadecyl ether (e.g., Brij® S20 polyethylene glycol octadecyl ether), seed oil surfactant (e.g., Ecosurf™ SA-15 seed oil surfactant), poloxamer 188 (a copolymer of polyoxyethylene and polyoxypropylene), nonylphenol ethoxylate (e.g., Tergitol™ NP-10 nonylphenol ethoxylate), and combinaitons thereof. In some embodiments, the non-ionic surfactant is selected from the group consisting of TWEEN 60 nonionic detergent, PPG-PEG-PPG Pluronic 10R5, Pluronic F-68 (PF 68), Polyoxyethylene (18) tridecyl ether, Polyoxyethylene (12) tridecyl ether, MERPOL SH surfactant, MERPOL OJ surfactant, MERPOL HCS surfactant, Poloxamer P188, Poloxamer P407, Poloxamer P 338, IGEPAL CO-720, IGEPAL CO-630, IGEPAL CA-720, Brij S20, BrijS10, Brij 010, Brij C10, BRIJ 020, ECOSURF EH-9, ECOSURF EH-14, TERGITOL 15-S-7, ECOSURF SA-15, TERGITOL15-S-9, TERGITOL 15-S-12, TERGITOL L-64, TERGITOLNP-7, TERGITOL NP-8, TERGITOL NP-9, TERGITOL NP-9.5, TERGITOL NP-10, TERGITOL NP-11, TERGITOL NP-12, TERGITOLNP-13, polysorbate 20, and any combinations thereof. In some embodiments, the non-ionic surfactant is Poloxamer P 188, Poloxamer P407, Pluronic F-68, Ecosurf SA-15, Brij S20, Tergitol NP-10, IGEPAL CA 720 or Tween 80. In some embodiments, the composition is substantially free of a non-ionic surfactant. In some embodiments, the non-ionic surfactant is not a polysorbate, e.g., Tween 80 (also referred to as polysorbate 80 or PS80).

When present, the non-ionic surfactant can be present at a concentration from about 0.0001% (w/v) to about 0.01% (w/v). For example, the composition can comprise a non-ionic surfactant at a concentration from about 0.0005% (w/v) to about 0.0015% (w/v). In some embodiments, the composition can comprise a non-ionic surfactant at a concentration of about 0.0001% (w/v), about 0.0002% (w/v), about 0.0003% (w/v), about 0.0004% (w/v), about 0.0005% (w/v), about 0.0006% (w/v), about 0.0007% (w/v), about 0.0008% (w/v), about 0.0009% (w/v), about 0.001% (w/v), about 0.002% (w/v), about 0.003% (w/v), about 0.004% (w/v), about 0.005% (w/v), about 0.006% (w/v), about 0.007% (w/v), about 0.008% (w/v), about 0.009% (w/v), or about 0.01%. (w/v). In some preferred embodiments, the composition comprises a non-ionic surfactant at a concentration of about 0.0005% (w/v) or about 0.001% (w/v).

In some embodiments, the composition comprises, in addition to the rAAV, a buffer (e.g., PBS, Tris·HCl, phosphate, citric acid, histidine, tromethamine, succinic acid, malic acid, α-ketoglutaric acid, carbonate buffer), a bulking agent (e.g., a polyol such as sorbitol, mannitol, glycerol, propylene glycol, polyethylene glycol, dulcitol, sucrose, lactose, maltose, trehalose and dextran) and a non-ionic surfactant (e.g., Poloxamer P 188, Poloxamer P407, Pluronic F-68, Ecosurf SA-15, Brij S20, Tergitol NP-10, IGEPAL CA 720 or Tween 80).

In some embodiments, the composition comprises, in addition to the rAAV, a buffer (e.g., PBS, Tris·HCl, phosphate, citric acid, histidine, tromethamine, succinic acid, malic acid, α-ketoglutaric acid, carbonate buffer), a bulking agent (e.g., a polyol such as sorbitol, mannitol, glycerol, propylene glycol, polyethylene glycol, dulcitol, sucrose, lactose, maltose, trehalose and dextran), a non-ionic surfactant (e.g., Poloxamer P 188, Poloxamer P407, Pluronic F-68, Ecosurf SA-15, Brij S20, Tergitol NP-10, IGEPAL CA 720 or Tween 80), and a multivalent ion (e.g., a multivalent ion selected from the group consisting of calcium, citrate, sulfate, and magnesium).

In some embodiments, the composition comprises, in addition to the rAAV, a buffer (e.g., PBS, Tris·HCl, phosphate, citric acid, histidine, tromethamine, succinic acid, malic acid, α-ketoglutaric acid, carbonate buffer), a bulking agent (e.g., a polyol such as sorbitol, mannitol, glycerol, propylene glycol, polyethylene glycol, dulcitol, sucrose, lactose, maltose, trehalose and dextran), and a multivalent ion (e.g., a multivalent ion selected from the group consisting of calcium, citrate, sulfate, and magnesium).

It is noted that any one of the specific buffers or group of buffers listed in the description of the compositions can be used with any one of the specific bulking agents or group of bulking agents listed in the description of the compositions and with any of the specific non-ionic surfactants or group of surfactants listed in the description of the compositions and with any of the specific multivalent ions and multivalent ion group listed in the description of the compositions. Similarly, any one of the specific bulking agents or group of bulking agents listed in the description of the compositions can be used with any one of the specific buffers or group of buffers listed in the description of the compositions and with any of the specific non-ionic surfactants or group of surfactants listed in the description of the compositions and with any of the specific multivalent ions and multivalent ion group listed in the description of the compositions. Likewise, any of the specific non-ionic surfactants or group of surfactants listed in the description of the compositions can be used with any one of the specific buffers or group of buffers listed in the description of the compositions and with any one of the specific bulking agents or group of bulking agents listed in the description of the compositions and with any of the specific multivalent ions and multivalent ion group listed in the description of the compositions. As well, any of the specific multivalent ions and multivalent ion group listed in the description of the compositions can be used with any one of the specific buffers or group of buffers listed in the description of the compositions and with any one of the specific bulking agents or group of bulking agents listed in the description of the compositions and with any of the specific non-ionic surfactants or group of surfactants listed in the description of the compositions. In other words, all individual specific combinations of buffers, buffer group, bulking agents, bulking agent groups, non-ionic surfactants, non-ionic surfactant groups, multivalent ions and multivalent ion groups listed in the description of the compositions are specifically contemplated.

### Exemplary Compositions

In some embodiments, the composition, e.g., the pharmaceutical composition comprises, in addition to the rAAV comprising human GAA (hGAA) e.g, AAV8-LSP-hGAA, about 10 mM Phosphate pH 7.4, about 200 mM NaCl, about 5 mM KCl, about 1% (w/v) mannitol, and about 0.0005% (w/v) IGEPAL CA 720. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises, in addition to the rAAV comprising human GAA (hGAA) e.g, AAV8-LSP-hGAA, about 20 mM Phosphate pH 7.4, about 300 mM NaCl, about 3 mM KCl, about 3% (w/v) mannitol, and about 0.001% (w/v) Brij S20. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises, in addition to the rAAV comprising human GAA (hGAA) e.g, AAV8-LSP-hGAA, about 20 mM Phosphate pH 7.4, about 300 mM NaCl, about 3 mM KCl, about 3% (w/v) sorbitol, and about 0.001% (w/v) Ecosurf SA-15. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises, in addition to the rAAV comprising human GAA (hGAA) e.g, AAV8-LSP-hGAA, about 10 mM Phosphate pH 7.4, about 350 mM NaCl, about 2.7 mM KCl, about 5% (w/v) sorbitol, and about 0.001% (w/v) poloxamer 188. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises, in addition to the rAAV comprising human GAA (hGAA) e.g, AAV8-LSP-hGAA, about 10 mM Phosphate pH 6.95-7.2, about 137 mM NaCl, about 2.7 mM KCl, about 0.9 mM CaCl₂, about 0.5 mM MgCl₂, and about 0.001% (w/v) Pluronic F-68. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises, in addition to the rAAV comprising human GAA (hGAA) e.g, AAV8-LSP-hGAA, about 10 mM Phosphate pH 7.3, about 180 mM NaCl, about 2.7 mM KCl, about 5% (w/v) sorbitol, and about 0.001% (w/v) Poloxamer 188. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises, in addition to the rAAV comprising human GAA (hGAA) e.g, AAV8-LSP-hGAA, about 15 mM Phosphate pH 7.4, about 375 mM NaCl, about 3.5 mM KCl, about 5% (w/v) sorbitol, and about 0.0005% (w/v) Tergitol NP-10. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises, in addition to the rAAV comprising human GAA (hGAA) e.g, AAV8-LSP-hGAA, about 15 mM Phosphate pH 7.4, about 375 mM NaCl, about 3.5 mM KCl, about 3% (w/v) glycerol, and about 0.0005% (w/v) Tween 80. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises, in addition to the rAAV comprising human GAA (hGAA) e.g, AAV8-LSP-hGAA, about 10 mM Phosphate pH 7.6, about 137 mM NaCl, about 2.7 mM KCl, about 5% (w/v) sorbitol, and about 0.010% Pluronic F-68. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises, in addition to the rAAV comprising human GAA (hGAA) e.g, AAV8-LSP-hGAA, about 10 mM Phosphate pH 7.4, about 137 mM NaCl, about 2.7 mM KCl, about 5% (w/v) sorbitol, about 0.01% Pluronic F-68, and about 20 mM MgSO₄. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises, in addition to the rAAV comprising human GAA (hGAA) e.g, AAV8-LSP-hGAA, about 10 mM Phosphate pH 7.6, about 137 mM NaCl, about 2.7 mM KCl, about 5% (w/v) mannitol, and about 0.010% Pluronic F-68. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises, in addition to the rAAV comprising human GAA (hGAA) e.g, AAV8-LSP-hGAA, about 10 mM Phosphate pH 7.3, about 137 mM NaCl, about 2.7 mM KCl, about 5% (w/v) mannitol, about 0.01% Pluronic F-68, and about 20 mM MgSO₄. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises, in addition to the rAAV comprising human GAA (hGAA) e.g, AAV8-LSP-hGAA, about 10 mM Phosphate pH 7.4, about 137 mM NaCl, about 2.7 mM KCl, about 5% (w/v) sorbitol, and about 20 mM MgSO₄. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises, in addition to the rAAV comprising human GAA (hGAA) e.g, AAV8-LSP-hGAA, about 10 mM Phosphate pH 7.4, about 137 mM NaCl, about 2.7 mM KCl, about 5% (w/v) mannitol, and about 20 mM MgSO₄. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises recombinant AAV vector (rAAV) comprising human GAA (hGAA) e.g, AAV8-LSP-hGAA, in 10 mM Phosphate pH 7.4, 200 mM NaCl, 5 mM KCl, 1% (w/v) mannitol, 0.0005% (w/v) IGEPAL CA 720 to a fill volume of 5 ml. In some embodiments, the fill volume is 1 ml, 2 ml, 3 ml, 4 ml, 5 ml, 6 ml, 7 ml, 8 ml, 9 ml, or, 10 ml. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises recombinant AAV vector (rAAV) comprising human GAA (hGAA) e.g, AAV8-LSP-hGAA, in 20 mM Phosphate pH 7.4, 300 mM NaCl, 3 mM KCl, 3% (w/v) mannitol, 0.001% (w/v) Brij S20 to a fill volume of 5 ml. In some embodiments, the fill volume is 1 ml, 2 ml, 3 ml, 4 ml, 5 ml, 6 ml, 7 ml, 8 ml, 9 ml, or, 10 ml. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises recombinant AAV vector (rAAV) comprising human GAA (hGAA) e.g, AAV8-LSP-hGAA, in 20 mM Phosphate pH 7.4, 300 mM NaCl, 3 mM KCl, 3% (w/v) sorbitol, 0.001% (w/v) Ecosurf SA-15 to a fill volume of 5 ml. In some embodiments, the fill volume is 1 ml, 2 ml, 3 ml, 4 ml, 5 ml, 6 ml, 7 ml, 8 ml, 9 ml, or, 10 ml. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises recombinant AAV vector (rAAV) comprising human GAA (hGAA) e.g, AAV8-LSP-hGAA, in 10 mM Phosphate pH 7.4, 350 mM NaCl, 2.7 mM KCl, 5% (w/v) sorbitol, 0.001% (w/v) poloxamer 188 to a fill volume of 5 ml. In some embodiments, the fill volume is 1 ml, 2 ml, 3 ml, 4 ml, 5 ml, 6 ml, 7 ml, 8 ml, 9 ml, or, 10 ml. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises recombinant AAV vector (rAAV) comprising human GAA (hGAA) e.g, AAV8-LSP-hGAA, in 15 mM Phosphate pH 7.4, 375 mM NaCl, 3.5 mM KCl, 5% (w/v) sorbitol, 0.0005% (w/v) Tergitol NP-10 to a fill volume of 5 ml. In some embodiments, the fill volume is 1 ml, 2 ml, 3 ml, 4 ml, 5 ml, 6 ml, 7 ml, 8 ml, 9 ml, or, 10 ml. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

In some embodiments, the composition, e.g., the pharmaceutical composition comprises recombinant AAV vector (rAAV) comprising human GAA (hGAA) e.g, AAV8-LSP-hGAA, in 15 mM Phosphate pH 7.4, 375 mM NaCl, 3.5 mM KCl, 3% (w/v) glycerol, 0.0005% (w/v) Tween 80 to a fill volume of 5 ml. In some embodiments, the fill volume is 1 ml, 2 ml, 3 ml, 4 ml, 5 ml, 6 ml, 7 ml, 8 ml, 9 ml, or, 10 ml. The particle concentration of rAAV as described herein is from about 1.0×10¹³ vg/mL to about 1.0×10¹⁴ vg/mL, or, from about 2.0×10¹³ vg/ml to about 1.0×10¹⁴ vg/mL, or, from about 2.5×10¹³ to about 1.0×10¹⁴ vg/mL.

Additional exemplary compositions/compositions comprising rAAV are described in International Publication number WO2022159679, the content of which is incorporated herein by reference in its entirety.

All compositions may be stored at −60° C. or, below.

### II. Subjects Amenable to Treatment

### A. Pompe Disease

The recombinant AAV expressing GAA protein as disclosed herein can be used in methods to treat Pompe disease. Pompe disease is a rare genetic disorder caused by a deficiency in the enzyme acid alpha-glucosidase (GAA), which is needed to break down glycogen, a stored form of sugar used for energy. Pompe disease is also known as glycogen storage disease type II, GSD II, type II glycogen storage disease, glycogenosis type II, acid maltase deficiency, alpha-1,4-glucosidase deficiency, cardiomegalia glycogenic diffusa, and cardiac form of generalized glycogenosis. The build-up of glycogen causes progressive muscle weakness (myopathy) throughout the body and affects various body tissues, particularly in the heart, skeletal muscles, liver, respiratory and nervous system.

Glycogen storage disease type II, also referred to as Pompe disease, is a rare disorder of metabolism inherited in an autosomal recessive manner, caused by deficiency of the lysosomal enzyme acid α-glucosidase (GAA). This disorder leads to the accumulation of lysosomal glycogen and destruction of skeletal, smooth and cardiac muscle. Pompe disease ranges in severity from a severe, infantile-onset myopathy accompanied by severe hypotonia and hypertrophic cardiomyopathy (infantile-onset Pompe disease [IOPD]) to a late-onset myopathy (LOPD). The defect in GAA can vary from complete to partial deficiency of GAA, which correlates with clinical severity. LOPD presents with proximal leg weakness and in some cases respiratory insufficiency without significant cardiac involvement and may progress to fatal respiratory failure.

Early onset (or the infantile form, IOPD) is the result of complete or near complete deficiency of GAA. Symptoms begin in the first months of life and progress rapidly, with feeding problems, poor weight gain, muscle weakness, floppiness, and head lag. Respiratory difficulties are often complicated by lung infections. The heart is grossly enlarged. Many infants with Pompe disease also have enlarged tongues. If untreated with Lumizyme, most babies die from cardiac or respiratory complications before their first birthday.

Late onset (or juvenile/adult, LOPD) Pompe disease is the result of a partial deficiency of GAA. The onset can be as early as the first decade of childhood or as late as the sixth decade of adulthood and is therefore characterized as slowly progressive. The primary symptom is proximal muscle weakness progressing to respiratory weakness and death from respiratory failure after a course lasting several years. The heart is usually not involved.

The presenting clinical manifestations of Pompe disease can vary widely depending on the age of disease onset and residual GAA activity. Residual GAA activity correlates with both the amount and tissue distribution of glycogen accumulation as well as the severity of the disease. Infantile-onset Pompe disease (less than 1% of normal GAA activity) is the most severe form and is characterized by hypotonia, generalized muscle weakness, and hypertrophic cardiomyopathy, and massive glycogen accumulation in cardiac and other muscle tissues. Death usually occurs within one year of birth due to cardiorespiratory failure. Juvenile-onset (1-10% of normal GAA activity) and adult-onset (10-40% of normal GAA activity) Pompe disease are more clinically heterogeneous, with greater variation in age of onset, clinical presentation, and disease progression. Juvenile- and adult-onset Pompe disease are generally characterized by lack of severe cardiac involvement, later age of onset, and slower disease progression, but eventual respiratory or limb muscle involvement results in significant morbidity and mortality. While life expectancy can vary, death generally occurs due to respiratory failure.

In any embodiment of the methods as disclosed herein, a GAA enzyme suitable for treating Pompe disease includes a wild-type human GAA, or a fragment or sequence variant thereof which retains the ability to cleave al-4 linkages in linear oligosaccharides. In some embodiments of the methods and compositions as disclosed herein, the GAA protein encoded by a wild type GAA nucleic acid sequence, e.g., SEQ ID NO: 11 or SEQ ID NO: 72 as disclosed in International Application WO2021102107, which is incorporated herein in its reference. In some embodiments of the methods and compositions as disclosed herein, the GAA protein is encoded by a codon optimized GAA nucleic acid sequence, for example, for any one or more of: (1) enhanced expression in vivo, (2) to reduce CpG islands or (3) reduce the innate immune response. In some embodiments of the methods and compositions as disclosed herein, the GAA protein is encoded by a codon optimized GAA nucleic sequence, for example, any nucleic acid sequence selected from any of: SEQ ID NO: 73, SEQ ID NO: 74, SEQ ID NO: 75 and SEQ ID NO: 76 or SEQ ID NO: 182 as disclosed in International Application WO2021102107, which is incorporated herein in its reference, or a nucleic acid sequence having at least 60%, or 70%, or 80%, 85% or 90% or 95%, or 98%, or 99% sequence identity to SEQ ID NO: 73, SEQ ID NO: 74, SEQ ID NO: 75, SEQ ID NO: 76 or SEQ ID NO: 182 as disclosed in International Application WO2021102107, which is incorporated herein in its reference.

In some embodiments of the methods and compositions as disclosed herein, a rAAV vector as described herein transduces the liver of a subject and secretes the hGAA polypeptide into the blood, which perfuses patient tissues where the hGAA polypeptide, is taken up by cells and transported to the lysosome, where the GAA enzyme acts to eliminate material that has accumulated in the lysosomes due to the enzyme deficiency. For lysosomal enzyme replacement therapy to be effective, the therapeutic enzyme must be delivered to lysosomes in the appropriate cells in tissues where the storage defect is manifest.

In some embodiments, upon administration, the AAV vector selectively expresses and secretes GAA from transduced hepatocytes. The primary mechanism of action of a AAV vector expressing hGAA polypeptide as disclosed herein is to secrete continuous low levels of endogenous GAA from the liver into the systemic circulation in order to provide therapeutic exposure levels of GAA to tissue (e.g., the muscle, but not exclusively the muscle), resulting in glycogen removal and restoration of cellular architecture and function.

B. Increasing GAA Activity in a Subject with Pompe Disease

In any embodiment of the methods as disclosed herein, administration of a AAV vector expressing GAA results in delivery of the GAA to muscle, and can be by any suitable method including intravenous administration, intra-arterial administration, and/or intra-peritoneal administration. Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). The most suitable route in any given case will depend on the nature and severity of the condition being treated and/or prevented and on the nature of the particular vector that is being used.

In some embodiments of one or more aspects of the invention, the AAV vector expressing GAA is administered by IV catheter into the cephalic vein. In some embodiment the composition containing the AAV vector is infused in a volume of 20 mL over approximately 30 minutes. The infusion can be preceded by a 10 mL flush with lactated Ringer's solution and can be further followed by a 40 mL flush with lactated Ringer's solution. Flush and vector can be administered at the rate of 60 mL/hour.

In any embodiment of the methods as disclosed herein, administration of a AAV vector expressing GAA as disclosed herein is to skeletal muscle according to the present invention, and includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles that can be injected are disclosed in International Application WO2021102107, which is incorporated herein its entirety by reference.

In any embodiment of the methods as disclosed herein, the rAAV vectors and/or rAAV genome are administered to the skeletal muscle, liver, diaphragm, costal, and/or cardiac muscle cells of a subject. For example, a conventional syringe and needle can be used to inject a rAAV virion suspension into an animal. Parenteral administration of a the rAAV vectors and/or rAAV genome, by injection can be performed, for example, by bolus injection or continuous infusion. Formulations for injection may be presented in unit dosage form, for example, in ampoules or in multi-dose containers, with an added preservative. The compositions may take such forms as suspensions, solutions or emulsions in oily or aqueous vehicles, and may contain agents for a pharmaceutical formulation, such as suspending, stabilizing and/or dispersing agents. Alternatively, the rAAV vectors and/or rAAV genome as disclosed herein can be in powder form (e.g., lyophilized) for constitution with a suitable vehicle, for example, sterile pyrogen-free water, before use.

In particular embodiments, more than one administration (e.g., two, three, four, five, six, seven, eight, nine, 10, etc., or more administrations) may be employed to achieve the desired level of GAA expression over a period of various intervals, e.g., hourly, daily, weekly, monthly, yearly, etc. Dosing can be single dosage or cumulative (serial dosing), and can be readily determined by one skilled in the art. As disclosed herein, it is envisioned that treatment of Pompe Disease according to the methods as disclosed herein comprises a one-time administration of an effective dose of a pharmaceutical composition comprising a AAV vector encoding a GAA polypeptide.

However, in alternative embodiments, treatment of a subject with Pompe disease may comprise multiple administrations of a pharmaceutical composition comprising a AAV vector encoding a GAA polypeptide when the subject is not administered long-term ERT, where the multiple administrations can be carried out over a range of time periods, such as, e.g., once yearly, or every 6-months, or about every 2-years, or about every 3-years, or about every 4 years, or about every 5-years or longer than 5-year intervals. The timing of administration can vary from individual to individual, depending upon such factors as the severity of an individual's symptoms. For example, in some embodiments, an effective dose of a AAV vector encoding a GAA polypeptide as disclosed herein can be administered to an individual once every year, or once every two years, or every six months for an indefinite period of time, or until the individual no longer requires therapy. A person of ordinary skill in the art will recognize that the condition of the individual can be monitored throughout the course of treatment and that the effective amount of a AAV vector encoding a GAA polypeptide as disclosed herein that is administered can be adjusted accordingly.

Injectables comprising a AAV vector encoding a GAA polypeptide as disclosed herein can be prepared in conventional forms, either as liquid solutions or suspensions, solid forms suitable for solution or suspension in liquid prior to injection, or as emulsions. Alternatively, one may administer a AAV vector encoding a GAA polypeptide as disclosed herein in a local rather than systemic manner, for example, in a depot or sustained-release formulation. Further, the virus vector and/or virus capsid can be delivered adhered to a surgically implantable matrix (e.g., as described in U.S. Patent Publication No. US-2004-0013645-A1). In some embodiments, a AAV vector encoding a GAA polypeptide as disclosed herein can be administered to the lungs of a subject by any suitable means, optionally by administering an aerosol suspension of respirable particles comprised of the virus vectors and/or virus capsids, which the subject inhales. The respirable particles can be liquid or solid. Aerosols of liquid particles comprising the virus vectors and/or virus capsids may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the virus vectors and/or capsids may likewise be produced with any solid particulate medicament aerosol generator, by techniques known in the pharmaceutical art.

In some embodiments, a AAV vector encoding a GAA polypeptide as disclosed herein can be formulated in a solvent, emulsion or other diluent in an amount sufficient to dissolve an rAAV vector disclosed herein. In other aspects of this embodiment, the rAAV vectors and/or rAAV genome encoding GAA polypeptide as disclosed herein can herein may be formulated in a solvent, emulsion or a diluent in an amount of, e.g., less than about 90% (v/v), less than about 80% (v/v), less than about 70% (v/v), less than about 65% (v/v), less than about 60% (v/v), less than about 55% (v/v), less than about 50% (v/v), less than about 45% (v/v), less than about 40% (v/v), less than about 35% (v/v), less than about 30% (v/v), less than about 25% (v/v), less than about 20% (v/v), less than about 15% (v/v), less than about 10% (v/v), less than about 5% (v/v), or less than about 1% (v/v). In other aspects, the rAAV vectors and/or rAAV genome encoding a GAA polypeptide as disclosed herein can disclosed herein may comprise a solvent, emulsion or other diluent in an amount in a range of, e.g., about 1% (v/v) to 90% (v/v), about 1% (v/v) to 70% (v/v), about 1% (v/v) to 60% (v/v), about 1% (v/v) to 50% (v/v), about 1% (v/v) to 40% (v/v), about 1% (v/v) to 30% (v/v), about 1% (v/v) to 20% (v/v), about 1% (v/v) to 10% (v/v), about 2% (v/v) to 50% (v/v), about 2% (v/v) to 40% (v/v), about 2% (v/v) to 30% (v/v), about 2% (v/v) to 20% (v/v), about 2% (v/v) to 10% (v/v), about 4% (v/v) to 50% (v/v), about 4% (v/v) to 40% (v/v), about 4% (v/v) to 30% (v/v), about 4% (v/v) to 20% (v/v), about 4% (v/v) to 10% (v/v), about 6% (v/v) to 50% (v/v), about 6% (v/v) to 40% (v/v), about 6% (v/v) to 30% (v/v), about 6% (v/v) to 20% (v/v), about 6% (v/v) to 10% (v/v), about 8% (v/v) to 50% (v/v), about 8% (v/v) to 40% (v/v), about 8% (v/v) to 30% (v/v), about 8% (v/v) to 20% (v/v), about 8% (v/v) to 15% (v/v), or about 8% (v/v) to 12% (v/v).

In any embodiment of the methods as disclosed herein, a AAV vector encoding a GAA polypeptide can be an AAV of any serotype, including but not limited to encapsulated by any AAV3b capsid selected from: AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54) or AAV3bSASTG capsid (i.e., a AAV3b capsid comprising Q263A/T265 mutations), where SEQ ID NO: 44, 46, 48, 50, 52 and 54 are disclosed in International Application WO2021102107, which is incorporated herein in its reference.

To facilitate delivery of a AAV vector encoding a GAA polypeptide as disclosed herein, it can be mixed with a carrier or excipient. Carriers and excipients that might be used include saline (especially sterilized, pyrogen-free saline) saline buffers (for example, citrate buffer, phosphate buffer, acetate buffer, and bicarbonate buffer), amino acids, urea, alcohols, ascorbic acid, phospholipids, proteins (for example, serum albumin), EDTA, sodium chloride, liposomes, mannitol, sorbitol, and glycerol. USP grade carriers and excipients are particularly useful for delivery of virions to human subjects.

In addition to the formulations described previously, a AAV vector encoding a GAA polypeptide as disclosed herein can also be formulated as a depot preparation. Such long acting formulations may be administered by implantation (for example subcutaneously or intramuscularly) or by IM injection. Thus, for example, a rAAV vector and/or rAAV genome as disclosed herein may be formulated with suitable polymeric or hydrophobic materials (for example as an emulsion in an acceptable oil) or ion exchange resins, or as sparingly soluble derivatives.

In any embodiment of the methods as disclosed herein, the method is directed to treating Pompe Disease that results from a deficiency of GAA in a subject, wherein a AAV vector encoding a GAA polypeptide as disclosed herein is administered to a patient suffering from Pompe Disease, and following administration, GAA is secreted from cells in the liver and there is uptake of the secreted GAA by cells in skeletal muscle tissue, cardiac muscle tissue, diaphragm muscle tissue or a combination thereof, wherein uptake of the secreted GAA results in a reduction in lysosomal glycogen stores in the tissue(s), including but not limited to muscle. In some embodiments, a AAV vector encoding a GAA polypeptide as disclosed herein is encapsulated in a capsid, e.g., encapsulated by any AAV3b capsid selected from: AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), the sequences of which are disclosed in International Application WO2021102107, which is incorporated herein in its reference.

In a particular embodiment, at least about 1.6×10¹ to about 4.0×10¹² vg/kg will be administered per dose in a pharmaceutically acceptable carrier. In a further embodiment, dosages of the virus vector and/or capsid to be administered to a subject depend upon the mode of administration, the severity and type of Pompe disease (i.e., LOPD or IOPD) to be treated and/or prevented, the individual subject's condition, age and gender, and the particular virus vector or capsid, the nucleic acid encoding GAA polypeptide to be delivered, and the like, and can be determined in a routine manner. Exemplary doses for achieving therapeutic effects are titers of at least about 1.5×10¹¹ vg/kg, or at least about 1.5×10¹² vg/kg, or at least about 4.0×10¹² vg/kg. It is encompassed that the dose for achieving therapeutic effects as disclosed herein may also be determined by the strength of the liver specific promoter (LSP) operatively linked to the nucleic acid encoding the GAA polypeptide, as well as specific signal sequence, and ability of the cell to cleave the signal sequence when secreted from the cell. In contrast, the dose of the AAV encoding the GAA polypeptide as disclosed herein can be lower than about 1.6×10¹² when the liver specific promoter is stronger than the LPS used in the AAV8-LSPhGAA vector exemplified in the Examples herein, however, the dose of AAV should be titrated and determined based on the level of GAA expressed in the cell, as determined by transduction efficiency of the AAV capsid and the LSP, and the ability of the cell to secrete the expressed GAA polypeptide in order to avoid GAA accumulation in the transfected cell and any associated cell toxicity.

In another aspect, disclosed herein is a method of treating Pompe Disease by administering a nucleic acid encoding a GAA to a cell, comprising contacting the cell with a rAAV vector and/or rAAV genome as disclosed herein, under conditions for the nucleic acid to be introduced into the cell and expressed to produce GAA. In some embodiments, the cell is a cell in vivo. In some embodiments, the cell is a mammalian cell in vivo.

### C. Increasing Motoneuron Function in a Mammal

In any embodiment of the methods as disclosed herein, a AAV vector encoding a GAA polypeptide as disclosed herein is useful in methods to increase phrenic nerve activity in a mammal having Pompe disease and/or insufficient GAA levels. For example, a AAV vector encoding a GAA polypeptide as disclosed herein, e.g., a rAAV vector and/or rAAV genome encapsulated in a capsid, e.g., encapsulated by AAV8 or any AAV3b capsid selected from: AAV3b capsid (SEQ ID NO: 44); AAV3b265D capsid (SEQ ID NO: 46), AAV3b ST (S663V+T492V) capsid (SEQ ID NO: 48), AAV3b265D549A capsid (SEQ ID NO: 50); AAV3b549A capsid (SEQ ID NO: 52); AAV3bQ263Y capsid (SEQ ID NO: 54), sequences of which are disclosed in International Application WO2021102107, which is incorporated herein in its reference, can be administered to the central nervous system (e.g., neurons). In another embodiment, retrograde transport a AAV vector encoding a GAA polypeptide as disclosed herein from the diaphragm (or other muscle) to the phrenic nerve or other motor neurons can result in biochemical and physiological correction of Pompe disease. These same principles could be applied to other neurodegenerative disease.

In an embodiment, a rAAV capsid of the rAAV virion used to treat Pompe Disease is any of those listed in Table 1 as disclosed in International Applications WO2020/102645, and WO2020/102667, each of which are incorporated herein in their entirety, and includes any of AAV8 or AAV3, or AAV3b (including but not limited to AAV3b serotypes AAV3b265D, AAV3b265D549A, AAV3b549A, AAV3bQ263Y, AAV3bSASTG (i.e., a AAV3b capsid comprising Q263A/T265 mutations) serotypes) is capable of reducing any one or more of the symptoms of (i) the feeling of weakness in a patient's lower extremities, including, the legs, trunk and/or arms, (ii) a shortness of breath, a hard time exercising, lung infections, a big curve in the spine, trouble breathing while sleeping, an enlarged liver, an enlarged tongue and/or a stiff joint, (iii) in a patient suffering from Pompe Disease by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, an AAV GAA of any serotype is capable of reducing any one or more of the systems of (i) the feeling of weakness in a patient's lower extremities, including, the legs, trunk and/or arms, ii) a shortness of breath, a hard time exercising, lung infections, a big curve in the spine, trouble breathing while sleeping, an enlarged liver, an enlarged tongue and/or a stiff joint, (iii) in a patient suffering from Pompe Disease by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In any embodiment of the methods and compositions as disclosed herein, at least one symptom associated with Pompe Disease, or at least one adverse side effect associated with Pompe Disease are reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%, and the severity of at least one symptom associated with Pompe Disease, or at least one adverse side effect is reduced by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95%. In another embodiment, at least one symptom associated with Pompe Disease, or at least one adverse side effect associated with Pompe Disease is reduced by about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70%.

### III. Recombinant AAV Expressing GAA

In all aspects disclosed herein, a recombinant AAV (rAAV) vectors and constructs for rAAV for delivering a GAA polypeptide to a subject in the methods to treat Pompe Disease as disclosed herein are disclosed in International Patent Application WO2020102645 and WO2021102107 both of which are incorporated herein in their entirety by reference.

As disclosed herein, one aspect of the technology relates to a method to treat Pompe disease using a rAAV vector comprising a capsid, and within its capsid, a nucleotide sequence referred to as the “rAAV vector genome”. The rAAV vector genome (also referred to as “rAAV genome) includes multiple elements, including, but not limited to two inverted terminal repeats (ITRs, e.g., the 5′-ITR and the 3′-ITR), and located between the ITRs are additional elements, including a promoter, a heterologous gene encoding a GAA polypeptide and a poly-A tail.

In some embodiments, the rAAV genome disclosed herein comprises a 5′ ITR and 3′ ITR sequence, and located between the 5′ITR and the 3′ ITR, a promoter, e.g., a liver specific promoter sequence as disclosed herein, which operatively linked to a heterologous nucleic acid encoding a nucleic acid encoding an alpha-glucosidase (GAA) polypeptide. In one embodiment, the heterologous nucleic acid sequence can optionally further comprise one or more of the following elements: an intron sequence, a nucleic acid encoding a secretory signal peptide, and a poly A sequence.

### A. Alpha-Glucosidase (GAA) Polypeptide

The GAA gene (NM_000152.3) is approximately 18.3 kilobases (kb) long and contains 20 exons (Dasouki et al. 2014). Its complementary DNA has 2,859 nucleotides of coding sequence which encode the immature 952 amino acid enzyme. GAA is synthesized as a membrane bound, catalytically inactive (with respect to the natural substrate glycogen) precursor which is sequestered in the endoplasmic reticulum. It undergoes sugar chain modification in the Golgi complex, followed by transport into the (minor) secretory pathway, or into lysosomes where it is trimmed in a stepwise process at both the amino- and carboxyl-termini. Phosphorylation of mannose residues ensures efficient transport of the enzyme to the lysosomes via the mannose 6-phosphate receptor. In the lysosomes, GAA catalyzes the hydrolysis of α1→4 glucosidic linkages in glycogen in the low potential hydrogen (pH) environment to glucose. Specificity for the natural substrate (glycogen) is gained during its maturation.

Many normal allelic variants exist in GAA and are responsible for the three known alloenzymes (GAA1, GAA2, and GAA4). More than 450 mutations in GAA have been reported in individuals with Pompe disease. Nonsense mutations, large and small gene rearrangements, and splicing defects have been observed with many mutations being potentially specific to families, geographic regions, or ethnicities. Combinations of mutations that result in either complete or nearly complete absence of GAA enzyme activity (typically <1% of normal activity in skin fibroblasts) are seen more commonly in individuals with IOPD, whereas those combinations that allow partial enzyme activity (approximately 2-40% of normal activity in skin fibroblasts) typically have LOPD presentation. GAA mutations result in messenger RNA instability and/or severely truncated acid α-glucosidase or an enzyme with markedly decreased activity. Dysfunction or absence of GAA leads to the accumulation of glycogen in lysosomes and in the cytoplasm in multiple tissues, resulting in the destruction of skeletal, smooth and cardiac muscle. The effect of the enzyme deficiency may extend to vesicle systems that are linked to lysosomes and may also affect receptors, such as glucose transporter 4, that cycle through these organelles. Evidence has also shown a failure of productive autophagy and the progressive accumulation of autophagosomes that disrupt the contractile apparatus in muscle fibers, which correlated with a lack of correction of skeletal muscle during ERT.

Alpha-glucosidase (GAA) polypeptide is a member of family 31 of glycoside hydrolyases. Human GAA is synthesized as a 110 kDal precursor (Wisselaar et al. (1993) J. Biol. Chem. 268(3): 2223-31). The mature form of the enzyme is a mixture of monomers of 70 and 76 kDal (Wisselaar et al. (1993) J. Biol. Chem. 268(3): 2223-31). The precursor enzyme has seven potential glycosylation sites and four of these are retained in the mature enzyme (Wisselaar et al. (1993) J. Biol. Chem. 268(3): 2223-31). The proteolytic cleavage events which produce the mature enzyme occur in late endosomes or in the lysosome (Wisselaar et al. (1993) J. Biol. Chem. 268(3): 2223-31).

The rAAV vector genome can encode a GAA polypeptide can include, for example, amino acid residues 40-952 of human GAA, or a smaller portion, such as amino acid residues 40-790.

The C-terminal 160 amino acids are absent from the mature 70 and 76 kDal GAA polypeptide species. However, certain Pompe alleles resulting in the complete loss of GAA activity map to this region, for example Val949Asp (Becker et al. (1998) J. Hum. Genet. 62:991). The phenotype of this mutant indicates that the C-terminal portion of the protein, although not part of the 70 or 76 kDal species, plays an important role in the function of the protein. It has also been reported that the C-terminal portion of the protein, although cleaved from the rest of the protein during processing, remains associated with the major species (Moreland et al. (Nov. 1, 2004) J. Biol. Chem., Manuscript 404008200). Accordingly, the C-terminal residues could play a direct role in the catalytic activity of the protein, and/or may be involved in promoting proper folding of the N-terminal portions of the protein.

The native GAA gene encodes a precursor polypeptide which possesses a signal sequence and an adjacent putative trans-membrane domain, a trefoil domain (PFAM PF00088) which is a cysteine-rich domain of about 45 amino acids containing 3 disulfide linkages (Thim (1989) FEBS Lett. 250:85), the domain defined by the mature 70/76 kDal polypeptide, and the C-terminal domain. It has been reported that both the trefoil domain and the C-terminal domain are required for the production of functional GAA, and that it is possible that the C-terminal domain interacts with the trefoil domain during protein folding perhaps facilitating appropriate disulfide bond formation in the trefoil domain.

The GAA polypeptide is described in U.S. Pat. Nos. 5,962,313 and 6,537,785, which are incorporated herein in their entireties by reference. One of ordinary skill in the art can appreciate particular positions of GAA to which a secretory signal peptide (SS) can be fused. Accordingly, in one aspect the invention relates to a GAA fusion protein, where the SS is fused to amino acid 40, 68, 69, 70, 71, 72, 779, 787, 789, 790, 791, 792, 793, or 796 of human GAA of SEQ ID NO: 10, or a modified GAA protein of SEQ ID NO: 170-174 as disclosed in International Application WO2021102107, or a portion thereof.

In some embodiments of the methods and compositions as disclosed herein, the human GAA protein expressed by the AAV comprises amino acids of SEQ ID NO: 10, or fragments or variants thereof, for example a human GAA protein beginning at any of residues selected from: 40, 68, 69, 70, 71, 72, 779, 787, 789, 790, 791, 792, 793, or 796 of SEQ ID NO: 10 or SEQ ID NO: 170-174 as disclosed in International Application WO2021102107, which is incorporated herein in its reference. In some embodiments of the methods and compositions as disclosed herein, the human GAA protein expressed by the AAV comprises amino acids of SEQ ID NO: 10, or a protein at least 60%, or 70%, or 80%, 85% or 90% or 95%, or 98%, or 99% identical to SEQ ID NO: 10 or SEQ ID NO: 170-174 as disclosed in International Application WO2021102107, which is incorporated herein in its reference. In some embodiments of the methods and compositions as disclosed herein, the human GAA protein expressed by the AAV comprises amino acids is a human GAA protein beginning at any of residues selected from: 40, 68, 69, 70, 71, 72, 779, 787, 789, 790, 791, 792, 793, or 796 of SEQ ID NO: 10 or SEQ ID NO: 170-174 as disclosed in International Application WO2021102107, which is incorporated herein in its reference, or a protein at least 60%, or 70%, or 80%, 85% or 90% or 95%, or 98%, or 99% identical thereto. In some embodiments, the human GAA protein expressed by the AAV comprises amino acids beginning at any of residues selected from: 40, 68, 69, 70, 71, 72, 779, 787, 789, 790, 791, 792, 793, or 796 of any of SEQ ID NO: 170 (modGAA; H199R, R223H) or SEQ ID NO: 171 (modGAA; H199R, R223H, H201L) or a protein at least 60%, or 70%, or 80%, 85% or 90% or 95%, or 98%, or 99% identical thereto, where SEQ ID NOS: 170 and 171 are disclosed in International Application WO2021102107.

In some embodiments, the human GAA protein expressed by the AAV comprises a GAA polypeptide of SEQ ID NO: 600, or a protein at least 60%, or 70%, or 80%, 85% or 90% or 95%, or 98%, or 99% identical to SEQ ID NO: 600, or a fragment of SEQ ID NO: 600, wherein the fragment begins at any of residues selected from: 40, 68, 69, 70, 71, 72, 779, 787, 789, 790, 791, 792, 793, or 796 of any of SEQ ID NO: 600 (modGAA; H199R, R223H, V780I) or a protein at least 60%, or 70%, or 80%, 85% or 90% or 95%, or 98%, or 99% identical to SEQ ID NO: 600, wherein SEQ ID NO: 600 has the following amino acid sequence:

MGVRHPPCSHRLLAVCALVSLATAALLGHILLHDELLVPRELSGS
SPVLEETHPAHQQGASRPGPRDAQAHPGRPRAVPTQCDVPPNSRE
DCAPDKAITQEQCEARGCCYIPAKQGLQGAQMGQPWCFFPPSYPS
YKLENLSSSEMGYTATLTRTTPTFFPKDILTLRLDVMMETENRLH
ETIKDPANRRYEVPLETPRVHSRAPSPLYSVEFSEEPFGVIVHRQ
LDGRVLLNTTVAPLFFADQFLQLSTSLPSQYITGLAEHLSPLMLS
TSWTRITLWNRDLAPTPGANLYGSHPFYLALEDGGSAHGVELLNS
NAMDVVLQPSPALSWRSTGGILDVYIFLGPEPKSVVQQYLDVVGY
PFMPPYWGLGFHLCRWGYSSTAITRQVVENMTRAHFPLDVQWNDL
DYMDSRRDFTENKDGFRDFPAMVQELHQGGRRYMMIVDPAISSSG
PAGSYRPYDEGLRRGVFITNETGQPLIGKVWPGSTAFPDFTNPTA
LAWWEDMVAEFHDQVPFDGMWIDMNEPSNFIRGSEDGCPNNELEN
PPYVPGVVGGTLQAATICASSHQFLSTHYNLHNLYGLTEAIASHR
ALVKARGTRPFVISRSTFAGHGRYAGHWTGDVWSSWEQLASSVPE
ILQFNLLGVPLVGADVCGFLGNTSEELCVRWTQLGAFYPEMRNHN
SLLSLPQEPYSESEPAQQAMRKALTLRYALLPHLYTLFHQAHVAG
ETVARPLELEFPKDSSTWTVDHQLLWGEALLITPVLQAGKAEVTG
YFPLGTWYDLQTVPIEALGSLPPPPAAPREPAIHSEGQWVTLPAP
LDTINVHLRAGYIIPLQGPGLTTTESRQQPMALAVALTKGGEARG
ELFWDDGESLEVLERGAYTQVIFLARNNTIVNELVRVTSEGAGLQ
LQKVTVLGVATAPQQVLSNGVPVSNFTYSPDTKVLDICVSLLMGE
QFLVSWC*.

(i) Modified GAA (modGAA)

In some embodiments, the cognate leader sequence of GAA (i.e., SEQ ID NO: 175 or amino acids 1-27 of SEQ ID NO: 170 as disclosed in International Application WO2021102107) is replaced with a non-endogenous signal sequence (also referred to herein as a “leader sequence”) of SEQ ID NO: 176, or an IL2 wild type leader peptide (SEQ ID NO: 178), modified IL2 leader peptide (SEQ ID NO: 180) or leader peptides at least 90% sequence identity to SEQ ID Nos 176, 178 or 180, where SEQ ID Nos 176, 178 or 180 are disclosed in International Application WO2021102107, which is incorporated herein in its entirety.

In some embodiments, the modified human GAA protein comprises a polypeptide with at least one modification selected from: H199R, R223H, V780R, V780I, or H201L of SEQ ID NO: 10 as disclosed in International Application WO2021102107, which is incorporated herein in its entirety, or a variant of at least 80%, 90%, 95%, or 99% homology to at least 500, 550, 600, 650, 700, 750, 800, 850, or 900 amino acids of SEQ ID NO: 10 having at least one of these modification. In some embodiments, the modified human GAA protein comprises a polypeptide comprises at least two modifications selected from: H199R, R223H, V780R, V780I or H201L of SEQ ID NO: 10 as disclosed in International Application WO2021102107, or a variant of at least 80%, 90%, 95%, or 99% homology to at least 500, 550, 600, 650, 700, 750, 800, 850, or 900 amino acids of SEQ ID NO: 10 having at least two of these modification. In some embodiments, the modified human GAA protein comprises a polypeptide with three modifications selected from: H199R, R223H, V780R, V780I, and H201L of SEQ ID NO: 10 (GAA-H199R-H201L-R223H or GAA-H199R-H201L-V780R), or a variant of at least 80%, 90%, 95%, or 99% homology to at least 500, 550, 600, 650, 700, 750, 800, 850, or 900 amino acids of SEQ ID NO: 10 as disclosed in International Application WO2021102107 having these three modifications.

In some embodiments, the cognate GAA leader peptide (also referred to as “signal sequence”) of amino acids 1-27 of SEQ ID NO: 10 (i.e., MGVRHPPCSHRLLAVCALVSLATAALL, SEQ ID NO: 175 as disclosed in International Application WO2021102107) is replaced with a different signal peptide (leader peptide). For example, the cognate leader peptide of GAA (SEQ ID NO: 175) can be replaced with any of: (i) an IgG1 leader peptide (referred to herein as a “201 leader peptide” or “2011p” having an amino acid sequence of: MEFGLSWVFLVALLKGVQCE (SEQ ID NO: 176) encoded by nucleic acid sequence SEQ ID NO: 177, (ii) wtIL2 lp: MYRMQLLSCIALSLALVTNS (SEQ ID NO: 178) encoded by nucleic acid sequence SEQ ID NO: 179, or (iii) mutIL2 lp: MYRMQLLLLIALSLALVTNS (SEQ ID NO: 180) encoded by nucleic acid sequence SEQ ID NO: 181. In some embodiments, the cognate GAA leader peptide (SEQ ID NO: 175) remains present, and an additional signal peptide is added, e.g., any one or more of signal peptides AAT, FN1, an IgG1 leader peptide (referred to herein as a “201 leader peptide” or “201lp” having an amino acid sequence of: MEFGLSWVFLVALLKGVQCE (SEQ ID NO: 176) encoded by nucleic acid sequence SEQ ID NO: 177, (ii) wtIL2 lp: MYRMQLLSCIALSLALVTNS (SEQ ID NO: 178) encoded by nucleic acid sequence SEQ ID NO: 179, or (iii) mutIL2 lp: MYRMQLLLLIALSLALVTNS (SEQ ID NO: 180) encoded by nucleic acid sequence SEQ ID NO: 181 as disclosed in International Application WO2021102107.

In some embodiments, GAA is modified to add or remove glycosylation sites such as N-linked glycosylation sites, O-linked glycosylation sites or both. In certain embodiments, the addition or removal of glycosylation sites are achieved by N-terminal deletions, C-terminal deletions, internal deletions, random point mutagenesis, or, site directed mutagenesis. In some embodiments, the exemplary GAA modification involve addition of one or more Asparagine (Asn) residue/s or, one or more mutation to yield Asparagine (Asn) residue/s or, deletion of one or more Asparagine (Asn) residue/s. In certain embodiments, all or some of the N-linked, and/or, O-linked glycosylation sites present in GAA are mutated. In some embodiments, GAA modifications will yield information pertaining to the biological activity, physical structure and/or substrate binding potential of GAA.

### (ii) Nucleic Acid Encoding GAA

In some embodiments, the rAAV genome useful in the methods to treat Pompe Disease as disclosed herein comprises a heterologous nucleic acid sequence encoding a secretory signal peptide fused in frame to the 3′ terminus of a GAA nucleic acid sequence that encodes the entire GAA polypeptide (e.g., the N-terminal/catalytic and the C-terminal domain). For example, heterologous nucleic acid sequence encoding a secretory signal peptide is fused in frame to the 3′ terminus of a GAA nucleic acid sequence that encodes the 70 kDa and 76 kDa GAA polypeptides, such both polypeptides are expressed from the rAAV genome when the rAAV vector transduces a mammalian cell. In some embodiments, expression of the GAA nucleic acid can be driven by two promoters in the rAAV genome or by one promoter driving expression of a bicistronic construct.

In some embodiments of the methods and compositions as disclosed herein, the rAAV vector comprises a nucleic acid sequence encoding a GAA protein is a wild type GAA nucleic acid sequence, e.g., SEQ ID NO: 11 or SEQ ID NO: 72 or SEQ ID NO: 182 as disclosed in International Application WO2021102107, which is incorporated herein in its entirety by reference. In some embodiments of the methods and compositions as disclosed herein, the rAAV vector comprises a nucleic acid sequence encoding a GAA protein which is a codon optimized GAA nucleic acid sequence, for any one or more of (i) enhanced expression in vivo, (ii) to reduce CpG islands, (iii). to reduce the innate immune response. Exemplary codon optimized GAA nucleic sequences encompassed for use in the methods and rAAV compositions as disclosed herein can be selected from any of: SEQ ID NO: 73, SEQ ID NO: 74, SEQ ID NO: 75, SEQ ID NO: 76 or SEQ ID NO: 182 as disclosed in International Application WO2021102107, or a nucleic acid sequence having at least 60%, or 70%, or 80%, 85% or 90% or 95%, or 98%, or 99% sequence identity to SEQ ID NO: 73, SEQ ID NO: 74, SEQ ID NO: 75, SEQ ID NO: 76 or SEQ ID NO: 182.

### B. Secretory Signal Peptide

The native GAA signal peptide is not cleaved in the ER thereby causing native GAA polypeptide to be membrane bound in the ER (Tsuji et al. (1987) Biochem. Int. 15(5):945-952). Disruption of the membrane association of GAA can be accomplished by replacing the endogenous (or cognate) GAA signal peptide (and optionally adjacent sequences) with an alternate signal peptide for GAA.

Accordingly, in representative embodiments, the rAAV vector and rAAV genome useful in the methods to treat Pompe disease as disclosed herein further comprises a heterologous nucleic acid encoding a GAA polypeptide to be transferred to a target cell, attached to a heterologous nucleic acid sequence that encodes a secretory signal peptide in the place of the endogenous GAA signal peptide. The heterologous nucleic acid is operatively associated with the segment encoding the secretory signal peptide, such that upon transcription and translation a fusion polypeptide is produced containing the secretory signal sequence operably associated with (e.g., directing the secretion of) the GAA polypeptide.

In some embodiments, the AAV vector encodes a GAA polypeptide that comprises the endogenous GAA signal peptide (e.g., amino acids 1-27 of SEQ ID NO: 10 (also referred to as “innate GAA” or “cognate GAA” signal peptide). In some embodiments, the AAV vector encodes a GAA polypeptide that comprises the endogenous GAA signal peptide (e.g., amino acids 1-27 of SEQ ID NO: 10 as disclosed in International Application WO2021102107) (also referred to as “innate GAA” or “cognate GAA” signal peptide) and an additional heterologous (nonnative) signal sequence. In some embodiments, the GAA polypeptide that lacks the endogenous signal peptide of amino acids 1-27 of GAA is fused to a secretory signal. In some embodiments of the compositions and methods described herein, the secretory signal serves a general purpose of assisting the secretion of the GAA polypeptide from the liver cells into the blood, where it can travel and be targeted to the lysosomes of mammalian cells, for example, human cardiac and skeletal muscle cells, as described herein. In some embodiments, a heterologous secretory signal is selected from any of: a AAT signal peptide, a fibronectin signal peptide (FN1), a GAA signal peptide, or an active fragment of AAT, FN1 or GAA signal peptide having secretory signal activity.

In some embodiments, the secretory signal peptide is heterologous to (i.e., foreign or exogenous to) the polypeptide of interest. For example, a heterologous secretory signal peptide is a fibronectin secretory signal peptide, the polypeptide of interest is not fibronectin. In some embodiments, the secretory signal peptide is selected from any of: AAT signal peptide, a fibronectin signal peptide (FN1), or an active fragment of AAT, FN1 or GAA signal peptide having secretory signal activity. In alternative embodiments, the secretory signal peptide is not heterologous to GAA, i.e., the signal peptide is the GAA signal peptide (i.e., residues 1-27 of the native GAA polypeptide).

In some embodiments, the cognate GAA signal peptide of amino acids 1-27 of SEQ ID NO: 10 (i.e., MGVRHPPCSHRLLAVCALVSLATAALL, SEQ ID NO: 175) is replaced with a different or heterologous leader peptide. For example, the cognate leader peptide of GAA (SEQ ID NO: 175) can be replaced with any of the heterologous signal peptides selected from: (i) an IgG1 leader peptide (referred to herein as a “201 leader peptide” or “201lp” having an amino acid sequence of: MEFGLSWVFLVALLKGVQCE (SEQ ID NO: 176) encoded by nucleic acid sequence SEQ ID NO: 177, (ii) wtIL2 lp: MYRMQLLSCIALSLALVTNS (SEQ ID NO: 178) encoded by nucleic acid sequence SEQ ID NO: 179, or (iii) mutIL2 lp: MYRMQLLLLIALSLALVTNS (SEQ ID NO: 180) encoded by nucleic acid sequence SEQ ID NO: 181, or a leader peptide having at least 90% sequence identity to any of SEQ ID NOs 176, 178 or 180.

In general, the secretory signal peptide will be at the amino-terminus (N-terminus) of the GAA polypeptide (i.e., the nucleic acid segment encoding the secretory signal peptide is 5′ to the heterologous nucleic acid encoding the GAA peptide in the rAAV vector or rAAV genome as disclosed herein). Alternatively, the secretory signal may be at the carboxyl-terminus or embedded within the GAA polypeptide, as long as the secretory signal is operatively associated therewith and directs secretion of the GAA polypeptide or GAA fusion polypeptide of interest (either with or without cleavage of the signal peptide from the GAA polypeptide) from the cell.

The secretory signal is operatively associated with the GAA polypeptide is targeted to the secretory pathway. Alternatively stated, the secretory signal is operatively associated with the GAA polypeptide such that the GAA-polypeptide is secreted from the cell at a higher level (i.e., a greater quantity) than in the absence of the secretory signal peptide. In general, typically at least about 20%, 30%, 40%, 50%, 70%, 80%, 85%, 90%, 95% or more of the GAA-polypeptide is secreted from the cell when a signal peptide is attached as compared to in the absence of the attachment of a secretory signal peptide. In other embodiments, essentially all of the detectable polypeptide (alone and/or in the form of the fusion polypeptide) is secreted from the cell.

By the phrase “secreted from the cell”, the polypeptide may be secreted into any compartment (e.g., fluid or space) outside of the cell including but not limited to: the interstitial space, blood, lymph, cerebrospinal fluid, kidney tubules, airway passages (e.g., alveoli, bronchioles, bronchia, nasal passages, etc.), the gastrointestinal tract (e.g., esophagus, stomach, small intestine, colon, etc.), vitreous fluid in the eye, and the cochlear endolymph, and the like.

Accordingly, in some embodiments, a AAV expressing GAA useful in the methods to treat Pompe Disease as disclosed herein comprises a 5′ ITR and 3′ ITR sequence, and located between the 5′ITR and the 3′ ITR, a liver specific promoter operatively linked to a heterologous nucleic acid encoding a secretory peptide and nucleic acid encoding an alpha-glucosidase (GAA) polypeptide (i.e., the heterologous nucleic acid encodes a GAA polypeptide comprising a signal peptide-GAA polypeptide).

In alternative embodiments, a AAV expressing GAA useful in the methods to treat Pompe Disease as disclosed herein comprises a 5′ ITR and 3′ ITR sequence, and located between the 5′ITR and the 3′ ITR, a promoter operatively linked to a heterologous nucleic acid encoding a secretory peptide and nucleic acid encoding an alpha-glucosidase (GAA) polypeptide.

Generally, secretory signal peptides are cleaved within the endoplasmic reticulum and, in some embodiments, the secretory signal peptide is cleaved from the GAA polypeptide prior to secretion. It is not necessary, however, that the secretory signal peptide is cleaved as long as secretion of the GAA polypeptide from the cell is enhanced and the GAA polypeptide is functional. Thus, in some embodiments, the secretory signal peptide is partially or entirely retained.

In some embodiments, the rAAV genome, or an isolated nucleic acid as disclosed herein comprises a nucleic acid encoding a chimeric polypeptide comprising a GAA polypeptide operably linked to a secretory signal peptide, and the chimeric polypeptide is expressed and produced from a cell transduced with the rAAV vector and the GAA polypeptide is secreted from the cell. The GAA polypeptide can be secreted after cleavage of all or part of the secretory signal peptide. Alternatively, the GAA polypeptide can retain the secretory signal peptide (i.e., the secretory signal is not cleaved). Thus, in this context, the “GAA polypeptide” can be a chimeric polypeptide comprising the secretory peptide.

Numerous secreted proteins and sequences that direct secretion from the cell are known in the art, are disclosed in U.S. Pat. No. 9,873,868, which is incorporated herein in its entirety by reference. Exemplary secreted proteins (and their secretory signals) include but are not limited to: erythropoietin, coagulation Factor IX, cystatin, lactotransferrin, plasma protease C1 inhibitor, apolipoproteins (e.g., APO A, C, E), MCP-1, α-2-HS-glycoprotein, α-1-microgolubilin, complement (e.g., CIQ, C3), vitronectin, lymphotoxin-α, azurocidin, VIP, metalloproteinase inhibitor 2, glypican-1, pancreatic hormone, clusterin, hepatocyte growth factor, insulin, α-1-antichymotrypsin, growth hormone, type IV collagenase, guanylin, properdin, proenkephalin A, inhibin β (e.g., A chain), prealbumin, angiocenin, lutropin (e.g., β chain), insulin-like growth factor binding protein 1 and 2, proactivator polypeptide, fibrinogen (e.g., β chain), gastric triacylglycerol lipase, midkine, neutrophil defensins 1, 2, and 3, α-1-antitrypsin, matrix gla-protein, α-tryptase, bile-salt-activated lipase, chymotrypsinogen B, elastin, IG lambda chain V region, platelet factor 4 variant, chromogranin A, WNT-1 proto-oncogene protein, oncostatin M, β-neoendorphin-dynorphin, von Willebrand factor, plasma serine protease inhibitor, serum amyloid A protein, nidogen, fibronectin, rennin, osteonectin, histatin 3, phospholipase A2, cartilage matrix Protein, GM-CSF, matrilysin, neuroendocrine protein 7B2, placental protein 11, gelsolin, M-CSF, transcobalamin I, lactase-phlorizin hydrolase, elastase 2B, pepsinogen A, MIP 1-β, prolactin, trypsinogen II, gastrin-releasing peptide II, atrial natriuretic factor, secreted alkaline phosphatase, pancreatic α-amylase, secretogranin I, β-casein, serotransferrin, tissue factor pathway inhibitor, follitropin β-chain, coagulation factor XII, growth hormone-releasing factor, prostate seminal plasma protein, interleukins (e.g., 2, 3, 4, 5, 9, 11), inhibin (e.g., alpha chain), angiotensinogen, thyroglobulin, IG heavy or light chains, plasminogen activator inhibitor-1, lysozyme C, plasminogen activator, antileukoproteinase 1, statherin, fibulin-1, isoform B, uromodulin, thyroxine-binding globulin, axonin-1, endometrial α-2 globulin, interferon (e.g., alpha, beta, gamma), β-2-microglobulin, procholecystokinin, progastricsin, prostatic acid phosphatase, bone sialoprotein II, colipase, Alzheimer's amyloid A4 protein, PDGF (e.g., A or B chain), coagulation factor V, triacylglycerol lipase, haptoglobuin-2, corticosteroid-binding globulin, triacylglycerol lipase, prorelaxin H2, follistatin 1 and 2, platelet glycoprotein IX, GCSF, VEGF, heparin cofactor II, antithrombin-III, leukemia inhibitory factor, interstitial collagenase, pleiotrophin, small inducible cytokine A1, melanin-concentrating hormone, angiotensin-converting enzyme, pancreatic trypsin inhibitor, coagulation factor VIII, α-fetoprotein, α-lactalbumin, senogelin II, kappa casein, glucagon, thyrotropin beta chain, transcobalamin II, thrombospondin 1, parathyroid hormone, vasopressin copeptin, tissue factor, motilin, MPIF-1, kininogen, neuroendocrine convertase 2, stem cell factor procollagen al chain, plasma kallikrein keratinocyte growth factor, as well as any other secreted hormone, growth factor, cytokine, enzyme, coagulation factor, milk protein, immunoglobulin chain, and the like.

In some embodiments, other secretory signal peptides encoded by the rAAV genome and in the rAAV vector as disclosed herein can be selected from, but are not limited to, the secretory signal sequences from prepro-cathepsin L (e.g., GenBank Accession Nos. KHRTL, NP_037288; NP_034114, AAB81616, AAA39984, P07154, CAA68691; the disclosures of which are incorporated by reference in their entireties herein) and prepro-alpha 2 type collagen (e.g., GenBank Accession Nos. CAA98969, CAA26320, CGHU2S, NP_000080, BAA25383, P08123; the disclosures of which are incorporated by reference in their entireties herein) as well as allelic variations, modifications and functional fragments thereof (as discussed above with respect to the fibronectin secretory signal sequence). Exemplary secretory signal sequences include for preprocathepsin L (Rattus norvegicus, MTPLLLLAVLCLGTALA [SEQ ID NO: 27]; Accession No. CAA68691) and for prepro-alpha 2 type collagen (Homo sapiens, MLSFVDTRTLLLLAVTLCLATC [SEQ ID NO: 28]; Accession No. CAA98969). Also encompassed are longer amino acid sequences comprising the full-length secretory signal sequence from preprocathepsin L and prepro-alpha 2 type collagen or functional fragments thereof (as discussed above with respect to the fibronectin secretory signal sequence).

In some embodiments, the secretory signal peptide is derived in part or in whole from a secreted polypeptide that is produced by liver cells. In some embodiments, a secretory signal peptide can further be in whole or in part synthetic or artificial. Synthetic or artificial secretory signal peptides are known in the art, see e.g., Barash et al., “Human secretory signal peptide description by hidden Markov model and generation of a strong artificial signal peptide for secreted protein expression,” Biochem. Biophys. Res. Comm. 294:835-42 (2002); the disclosure of which is incorporated herein in its entirety. In particular embodiments, the secretory signal peptide comprises, consists essentially of, or consists of the artificial secretory signal: MWWRLWWLLLLLLLLWPMVWA (SEQ ID NO: 29) or variations thereof having 1, 2, 3, 4, or 5 amino acid substitutions (optionally, conservative amino acid substitutions, conservative amino acid substitutions are known in the art).

Exemplary signal peptides for use in the methods and compositions as disclosed herein can be selected from any signal peptide disclosed in Table 2, or functional variants thereof. Exemplary signal peptides are Fibronectin (FN1), or AAT. In some embodiments of the methods and compositions disclosed herein, the rAAV vector composition comprises the nucleic acid encoding a secretory signal peptide, e.g., encoding a secretory signal peptide selected from an AAT signal peptide (e.g., SEQ ID NO: 17), a fibronectin signal peptide (FN1) (e.g., SEQ ID NO: 18-21), a GAA signal peptide, an hIGF2 signal peptide (e.g., SEQ ID NO: 22) or an active fragment thereof having secretory signal activity, e.g., a nucleic acid encoding an amino acid sequence that has at least about 75%, or 80%, or 85%, or 90%, or 95%, or 98%, or 99% sequence identity to SEQ ID NOs: 17-22, where SEQ ID NO: 17-22 are disclosed in International Application WO2021102107, which is incorporated herein in its reference.

In some embodiments of the methods and compositions as disclosed herein, the nucleic acid encoding the secretory signal is selected from any of SEQ ID NO: 17, 81-21, 22-26, or a nucleic acid sequence at least about 75%, or 80%, or 85%, or 90%, or 95%, or 98%, or 99% sequence identity to any of SEQ ID NOs: 17 or 22-26, where SEQ ID NO: 17 and 22-26 are disclosed in International Application WO2021102107, which is incorporated herein in its reference

### Fibronectin Secretory Signal Peptide:

In some embodiments, the secretory signal peptide is a fibronectin secretory signal peptide, which term includes modifications of naturally occurring sequences (as described in more detail below).

In some embodiments, the secretory signal peptide is a fibronectin signal peptide, e.g., a signal sequence of human fibronectin or a signal sequence from rat fibronectin. Fibronectin (FN1) signal sequences and modified FN1 signal peptides encompassed for use in the rAAV genome and rAAV vectors described herein are disclosed in U.S. Pat. No. 7,071,172, which is incorporated herein in its entirety by reference, and in Table 3 of provisional application 62/937,556, filed on Nov. 19, 2019 or International Application WO2021102107, which is incorporated herein in its reference. Examples of exemplary fibronectin secretory signal sequences include, but are not limited to those listed in Table 1 of U.S. Pat. No. 7,071,172, which is incorporated herein in its entirety by reference.

TABLE 2
Exemplary Fibronectin (FN1) secretory signal peptides

| Species | Secretory Signal sequence | Nucleic acid sequence |
|---|---|---|
| H. Sapiens | MLRGPGPGLLLLAVQCLGTAV | ATG CTT AGG GGT CCG GGG CCC GGG CTG |
| | PSTGA (SEQ ID NO: 20) | CTG CTG CTG GCC GTC CAG TGC CTG GGG |
| | | ACA GCG GTG CCC TCC ACG GGA GCC |
| | | (SEQ ID NO: 25) |
| R. Norvegicus | MLRGPGPGRLLLLAVLCLGTSV | 5′- |
| | RCTETGKSKR | ATGCTCAGGGGTCCGGGACCCGGGCGGCT |
| | (SEQ ID NO: 18) | GCTGCTGCTAGCAGTCCTGTGCCTGGGGAC |
| | | ATCGGTGCGCTGCACCGAAACCGGGAAGA |
| | | GCAAGAGG-3 (SEQ ID NO: 23) |
| | | (nucleotides 208-303) |
| R. Norvegicus | MLRGPGPGRLLLLAVLCLGTSV | 5′-ATG CTC AGG GGT CCG GGA CCC GGG |
| | RCTETGKSKR ↑ LALQIV | CGG CTG CTG CTG CTA GCA GTC CTG TGC |
| | (SEQ ID NO: 19) | CTG GGG ACA TCG GTG CGC TGC |
| | | ACC GAA ACC GGG AAG AGC AAG AGG ↑ |
| | | CAG GCT CAG CAA ATC GTG-3′. (SEQ ID |
| | | NO: 24) (↑ denotes the cleavage site) |
| X. laevis | MRRGALTGLLLVLCLSVVLRA | ATG CGC CGG GGG GCC CTG ACC GGG CTG |
| | APSATSKKRR (SEQ ID NO: 21) | CTC CTG GTC CTG TGC CTG AGT GTT GTG |
| | | CTA CGT GCA GCC CCC TCT GCA ACA AGC |
| | | AAG AAG CGC AGG (SEQ ID NO: 26) |

### Peptidase Cleavage Sites

In some embodiments, one or more exogenous peptidase cleavage site may be inserted into the secretory signal peptide -GAA polypeptide, e.g., between the secretory signal peptide and the GAA polypeptide. In particular embodiments, an autoprotease (e.g., the foot and mouth disease virus 2A autoprotease) is inserted between the secretory signal peptide and the GAA polypeptide. In other embodiments, a protease recognition site that can be controlled by addition of exogenous protease is employed (e.g., Lys-Arg recognition site for trypsin, the Lys-Arg recognition site of the Aspergillus KEX2-like protease, the recognition site for a metalloprotease, the recognition site for a senne protease, and the like). Modification of the GAA polypeptide to delete or inactivate native protease sites is encompassed herein and disclosed in U.S. Provisional Application 62,937,556, filed on Nov. 19, 2019 and International Application WO2021102107, which is incorporated herein in its reference.

### C. Spacer and Fusion Junction of the GAA Polypeptide

Where GAA is expressed with a non-GAA secretory signal peptide (e.g., SS-GAA polypeptide), the signal peptide can be fused directly to the GAA polypeptide or can be separated from the GAA polypeptide by a linker. An amino acid linker (also referred to herein as a “spacer”) incorporates one or more amino acids other than that appearing at that position in the natural protein. Spacers can be generally designed to be flexible or to interpose a structure, such as an a-helix, between the two protein moieties.

Accordingly, in some embodiments of the methods and compositions disclosed herein, a recombinant AAV vector comprises a heterologous nucleic acid sequence encoding an GAA polypeptide, wherein the GAA protein further comprises a spacer comprising a nucleotide sequence of at least 1 amino acid in length, which is located N-terminal to the GAA polypeptide.

In one embodiment, the spacer at least 50% identical to the sequence GGGTVGDDDDK (SEQ ID NO: 35).

In some embodiments, a spacer or linker can be relatively short, e.g., at least 1, 2, 3, 4 or 5 amino acids, or such as the sequence Gly-Ala-Pro (SEQ ID NO: 31) or Gly-Gly-Gly-Gly-Gly-Pro (SEQ ID NO: 32), or can be longer, such as, for example, 5-10 amino acids in length or 10-25 amino acids in length. For example, flexible repeating linkers of 3-4 copies of the sequence (GGGGS (SEQ ID NO:33)) and a-helical repeating linkers of 2-5 copies of the sequence (EAAAK (SEQ ID NO:34)) have been described (Arai et al. (2004) Proteins: Structure, Function and Bioinformatics 57:829-838).

The use of another linker, GGGTVGDDDDK (SEQ ID NO: 35) has also been reported (DiFalco et al. (1997) Biochem. J. 326:407-413) and is encompassed for use. Linkers incorporating an a-helical portion of a human serum protein can be used to minimize immunogenicity of the linker region.

In some embodiments, the spacer is encoded by nucleic acids GGC GCG CCG (SEQ ID NO: 30) which encodes the amino acid spacer comprising amino acids GAP or Gly-Ala-Pro (SEQ ID NO: 31).

The site of a fusion junction in the GAA polypeptide to fuse with either the signal peptide (to generate a SS-GAA fusion protein) should be selected with care to promote proper folding and activity of each polypeptide in the fusion protein and to prevent premature separation of a signal peptide from a GAA polypeptide.

In some embodiments, a spacer has a helical structure. In another specific embodiment, a spacer is at least 50% identical to the sequence GGGTVGDDDDK (SEQ ID NO: 35). In some embodiments of the methods and compositions as disclosed herein, the spacer is SEQ ID NO: 31 (encoded by nucleic acids of SEQ ID NO: 30). In some embodiments of the methods and compositions as disclosed herein, the spacer is selected from any of: SEQ ID NO: 31, SEQ ID NO: 32, SEQ ID NO: 33, SEQ ID NO: 34 or SEQ ID NO: 35.

In some embodiments, a signal peptide can be fused, directly or by a spacer, to amino acid 40 or amino acid 70 of GAA, a position permitting expression of the protein, catalytic activity of the GAA protein, and proper secretion of the GAA polypeptide as described herein in the Examples. Alternatively, a signal sequence or signal peptide can be fused at or near the cleavage site separating the C-terminal domain of GAA from the mature polypeptide. This permits synthesis of a GAA protein with an internal signal peptide, which optionally can be cleaved to liberate the mature polypeptide or the C-terminal domain from the signal sequence, depending on placement of cleavage sites. Alternatively, the mature polypeptide can be synthesized as a fusion protein at about position 791 without incorporating C-terminal signal peptide sequences in the open reading frame of the expression construct.

In order to facilitate folding of the signal peptide, GAA amino acid residues adjacent to the fusion junction can be modified. For example, since it is possible that GAA cysteine residues may interfere with proper folding of the signal peptide, the terminal GAA cysteine 952 can be deleted or substituted with serine to accommodate a C-terminal signal peptide. The signal peptide can also be fused immediately preceding the final Cys952. The penultimate cys938 can be changed to proline in conjunction with a mutation of the final Cys952 to serine.

### D. CS Sequence

In some embodiments of the methods and compositions disclosed herein, a recombinant AAV vector comprises a heterologous nucleic acid sequence that further comprises at collagen stability (CS) sequence located 3′ of the nucleic acid encoding the GAA polypeptide and 5′ of the 3′ ITR sequence. In some embodiments, the rAAV genome disclosed herein comprises a heterologous nucleic acid sequence that can optionally comprise a Collagen stability sequence (CS or CSS), which is positioned 3′ of the GAA gene and 5′ of a polyA signal. In some embodiments, the CS sequence can be replaced by a 3′ UTR sequence as disclosed herein.

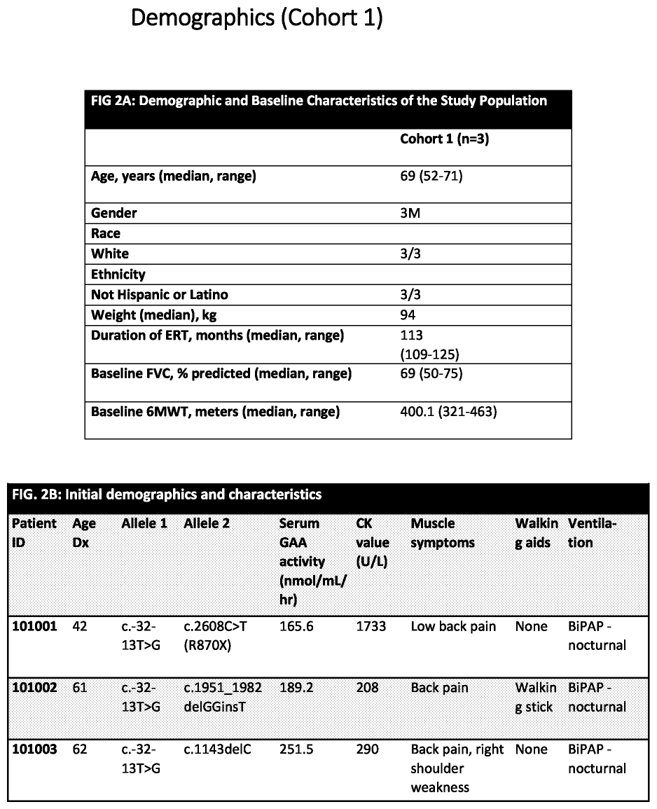

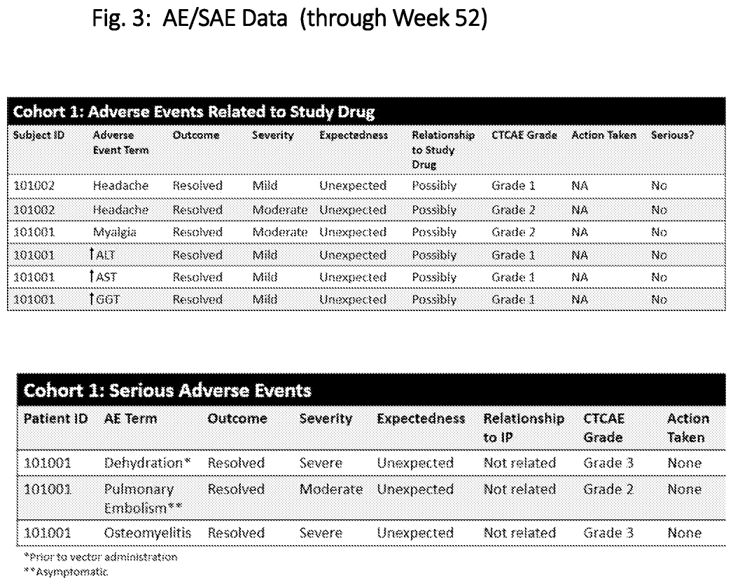

Exemplary collagen stability sequences include CCCAGCCCACTTTTCCCCAA (SEQ ID NO: 65) or a sequence at least 85%, 90%, 95%, 96%, 97%, 98% or 99% sequence identity thereto. An exemplary collagen stability sequence can have an amino acid sequence of P S P L F P (SEQ ID NO: 66) or an amino acid sequence having at least 85%, 90%, 95%, 96%, 97%, 98% or 99% sequence identity thereto. CS sequences are disclosed in Holick and Liebhaber, Proc. Nat. Acad. Sci. 94: 2410-2414, 1997 (See, e.g. FIG. 3, p. 5205), which is incorporated herein its entirety by reference.

### E. Liver Specific Promoters (LSP)

In some embodiments, to achieve appropriate levels of GAA expression, the rAAV genotype comprises a liver specific promoter (LSP). A LSP enables expression of the operatively linked gene in the liver, and can in some embodiments, be and inducible LSP. In an embodiment, a LSP is located upstream 5′ and is operatively linked to the heterologous nucleic acid sequence encoding the GAA protein. Exemplary liver-specific promoters useful in the AAV to treat Pompe according to the method disclosed herein are disclosed in International WO 2020102645 and WO2021102107, which are incorporated herein in their entirety by reference. Exemplary liver specific promoters include, but are not limited to, transthyretin promoter (TTR), LSP promoter (LSP), a synthetic liver specific promoter.

In some embodiments of the methods and compositions as disclosed herein, the promoter is a liver specific promoter (LSP), and can be selected from any liver specific promoters including, but not limited to, a transthyretin promoter (TTR), a Liver specific promoter (LSP), for example, as disclosed in U.S. Pat. No. 5,863,541 (TTR promoter), or LSP promoter (PNAS; 96: 3906-3910, 1999. See e.g. p. 3906, Materials and Methods, rAAV construction), a synthetic liver promoter, the references which are incorporated herein in their entireties by reference. Other liver promoters can be used, for example, synthetic liver promoters.

In some embodiments, the promoter is a LP1 promoter (which is SEQ ID NO: 432 as disclosed in WO2021102107, which is incorporated herein in its entirety by reference), or a variant having at least sequence at least 85%, 90%, 95%, 96%, 97%, 98% or 99% nucleotide sequence identity thereto.

In some embodiments, the TTR promoter is a truncated TTR promoter, e.g., comprising SEQ ID NO: 12 as disclosed in International WO 2020102645, which is incorporated herein in its entirety by reference, or a variant having at least sequence at least 85%, 90%, 95%, 96%, 97%, 98% or 99% nucleotide sequence identity thereto.

In some embodiments, the LSP is a TBG promoter, e.g., comprising SEQ ID NO: 435 as disclosed in International WO2021102107, which is incorporated herein in its entirety by reference, or a variant having at least sequence at least 85%, 90%, 95%, 96%, 97%, 98% or 99% nucleotide sequence identity thereto.

Other liver specific promoters include, but are not limited to promoters for the LDL receptor, Factor VIII, Factor IX, phenylalanine hydroxylase (PAH), ornithine transcarbamylase (OTC), and a 1-antitrypsin (hAAT), and HCB promoter. In Other liver specific promoters include the AFP (alpha fetal protein) gene promoter and the albumin gene promoter, as disclosed in EP Patent Publication 0 415 731, the a-1 antitrypsin gene promoter, as disclosed in Rettenger, Proc. Natl. Acad. Sci. 91 (1994) 1460-1464, the fibrinogen gene promoter, the APO-A1 (Apolipoprotein A1) gene promoter, and the promoter genes for liver transference enzymes such as, for example, SGOT, SGPT and g-glutamyle transferase. See also 2001/0051611 and PCT Patent Publications WO 90/07936 and WO 91/02805, which are incorporated herein in their entirety by reference. In some embodiments, the liver specific promoter is a recombinant liver specific promoter, e.g., as disclosed in US20170326256A1, which is incorporated herein in its entirety by reference.

In some embodiments, a liver specific promoter is the hepatitis B X-gene promoter and the hepatitis B core protein promoter. In some embodiments, liver specific promoters can be used with their respective enhancers. The enhancer element can be linked at either the 5′ or the 3′ end of the nucleic acid encoding the GAA polypeptide. The hepatitis B X gene promoter and its enhancer can be obtained from the viral genome as a 332 base pair EcoRV-NcoI DNA fragment employing the methods described in Twu, J Virol. 61 (1987) 3448-3453. The hepatitis B core protein promoter can be obtained from the viral genome as a 584 base pair BamHI-Bglll DNA fragment employing the methods described in Gerlach, Virol 189 (1992) 59-66. It may be necessary to remove the negative regulatory sequence in the BamHI-Bglll fragment prior to inserting it.

In some embodiments, a synthetic liver specific promoter is selected from any of: SEQ ID NOS: 86, 91-96, or 146-150, or 270-430 as disclosed in International Application WO2021102107 which is incorporated herein in its entirety by reference, or nucleic acid sequence that is at least 80%, or at least 90% or 95% identical thereto or to the source regulatory nucleic acid sequence.

In some embodiments, a liver-specific promoter (LSP) in a AAV expressing GAA useful in the methods to treat Pompe disease as disclosed herein comprises a nucleic acid sequence selected from any promoter listed from SEQ ID NOS: 86 (CRM 0412), SEQ ID NO: 91 (SP0412) or SEQ ID NO: 92 (SP0422), SEQ ID NOS: 93 (SP0239), SEQ ID NO: 94 (SP0265), SEQ ID NO: 95 (SP0240) or SEQ ID NO: 96 (SP0246), or SEQ ID NO: 146 (SP0265-UTR), SEQ ID NO: 147 (SP0239-UTR), SEQ ID NO: 148 (SP0240-UTR), SEQ ID NO: 149 (SP0246-UTR) or SEQ ID NO: 150 (SP0131-A1-UTR) as disclosed in International Application or a functional fragment or variant thereof, or any LSP selected from SEQ ID NO: 270-341 or 342-430, or a functional fragment or variant thereof, where SEQ ID NOS: 86, 91-96, or 146-150, or 270-430 are disclosed in International Application WO2021102107 which is incorporated herein in its entirety by reference.

In some embodiments, an rAAV vector genome can include one or more constitutive promoters, such as viral promoters or promoters from mammalian genes that are generally active in promoting transcription.

It is envisioned that the liver-specific promoter used to express the GAA polypeptide is selected in combination with, or in conjunction with the selection of the signal sequence. In particular, without wishing to be bound by theory, if a strong liver-specific promoter is selected, the signal sequence should be selected that is sufficient to secrete the expressed GAA out of the cell, in order to avoid GAA accumulation in the cell and any associated cell toxicity, and/or to avoid the generation of anti-GAA antibodies.

It is encompassed that the LSP is selected in conjunction with the signal sequence, so that the strength of the liver specific promoter (LSP) that is operatively linked to the nucleic acid encoding the GAA polypeptide can be counter-balanced with the ability of the cell to secrete the expressed GAA protein. Thus, if the liver specific promoter is strong, the specific signal sequence must be sufficiently effective to allow for the expressed GAA can be secreted from the cell so that GAA does not accumulate and create cell toxicity and/or induce an immune response. Thus, the cell secretory pathway, and the selected signal sequence must be able to match the level of GAA expressed by the AAV, where the level of GAA expression is dependent on both the AAV transduction efficiency (determined by AAV dose and capsid) and the strength of the liver specific promoter.

In some embodiments of the methods to treat Pompe disease disclosed herein, a synthetic liver-specific promoter useful in the AAV vector is disclosed in Table 4 of International Application WO2021102107, or any LSP promoter selected from SEQ ID NOS: 86, 91-96, 146-150 of International Application WO2021102107, or any LSP selected from SEQ ID NO: 270-341 or 342-430 of International Application WO2021102107, a synthetic liver-specific promoter is able to promote liver-specific transgene expression and has an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TTR promoter comprising SEQ ID NO: 431 as International Application WO2021102107.

In some embodiments of the methods to treat Pompe disease disclosed herein, a synthetic liver-specific promoter is selected from any of the LSP disclosed herein in Table 4 of International Application WO2021102107, or any LSP promoter selected from SEQ ID NOS: 86, 91-96, 146-150 disclosed in International Application WO2021102107, or any LSP selected from SEQ ID NO: 270-341 or 342-430 disclosed in International Application WO2021102107, where the synthetic liver-specific promoter is able to promote liver-specific transgene expression and has an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TBG promoter of SEQ ID NO: 435 as disclosed in International Application WO2021102107.

In some embodiments, a liver-specific promoter which is a functional variant of a given promoter element preferably retains at least 80% of its activity, more preferably at least 90% of its activity, more preferably at least 95% of its activity, and yet more preferably 100% of its activity (compared to the reference promoter comprising the unmodified promoter element). Suitable assays for assessing liver-specific promoter activity are disclosed in Examples 12 and 13 of International Application WO2021102107 which is incorporated herein in its entirety by reference.

### F. Regulatory Sequences and Intron Sequences

In some embodiments, the liver-specific promoters as set out above are operably linked to one or more additional regulatory sequences. An additional regulatory sequence can, for example, enhance expression compared to the liver-specific promoter which is not operably linked the additional regulatory sequence. Generally, it is preferred that the additional regulatory sequence does not substantively reduce the specificity of the liver-specific promoter.

For example, the liver-specific promoter can be operably linked to a sequence encoding a UTR (e.g. a 5′ and/or 3′ UTR), an intron, or such. In some embodiments, the liver-specific promoter is operably linked to sequence encoding a UTR, e.g. a 5′ UTR. A 5′ UTR can contain various elements that can regulate gene expression. The 5′ UTR in a natural gene begins at the transcription start site and ends one nucleotide before the start codon of the coding region. It should be noted that 5′ UTRs as referred to herein may be an entire naturally occurring 5′ UTR or it may be a portion of a naturally occurring 5′ UTR. The 5′ UTR can also be partially or entirely synthetic. In eukaryotes, 5′ UTRs have a median length of approximately 150 nt, but in some cases they can be considerably longer. Regulatory sequences that can be found in 5′ UTRs are disclosed in International Application WO2021102107 which is incorporated herein in its entirety by reference.

Introns within 5′ UTRs have been linked to regulation of gene expression and mRNA export. In some embodiments, a liver-specific promoter as set out above is operably linked to a sequence encoding a 5′ UTR derived from the CMV major immediate gene (CMV-IE gene). For example, the 5′ UTR from the CMV-IE gene suitably comprises the CMV-IE gene exon 1 and the CMV-IE gene exon 1, or portions thereof. In some cases, the promoter element may be modified in view of the linkage to the 5′ UTR, for example sequences downstream of the transcription start site (TSS) in the promoter element can be removed (e.g. replaced with the 5′ UTR).

The CMV-IE 5′UTR is described in Simari, et al, Molecular Medicine 4: 700-706, 1998 “Requirements for Enhanced Transgene Expression by Untranslated Sequences from the Human Cytomegalovirus Immediate-Early Gene”, which is incorporated herein by reference. Variants of the CMV-IE 5′ UTR sequences discussed in Simari, et al. are also set out in WO2002/031137, incorporated by reference, and the regulatory sequences disclosed therein can also be used. Other UTRs that can be used in combination with a promoter are known in the art, e.g. in Leppek, K., Das, R. & Bama, M. “Functional 5′ UTR mRNA structures in eukaryotic translation regulation and how to find them”. Nat Rev Mol Cell Biol 19, 158-174 (2018), incorporated by reference.

In some embodiments the sequence encoding the 5′ UTR comprises SEQ ID NO: 145 as disclosed in disclosed in International Application WO2021102107, or a functional variant thereof. In some embodiments, functional variants may have a sequence that is at least 60%, 65%, 70%, 75%, 80%, 85%, 90%, 95%, 96%, 97%, 98%, or 99% identical thereto. SEQ ID NO: 145 encodes a CMV-IE 5′ UTR.

In some embodiments the 5′ UTR comprises a nucleic acid motif that functions as the protein translation initiation site, e.g. sequences that define a Kozak sequence in the mRNA produced. For example, in some embodiments, the sequence encoding the 5′ UTR comprises the sequence motif GCCACC (SEQ ID NO: 153) at or near its 3′ end. Other Kozak sequences or other protein translation initiation sites can be used, as is known in the art (e.g. Marilyn Kozak, “Point Mutations Define a Sequence Flanking the AUG Initiator Codon That Modulates Translation by Eukaryotic Ribosomes” Cell, Vol. 44, 283-292, Jan. 31, 1986; Marilyn Kozak “At Least Six Nucleotides Preceding the AUG Initiator Codon Enhance Translation in Mammalian Cells” J. Mol. Rid. (1987) 196, 947-950; Marilyn Kozak “An analysis of 5′-noncoding sequences from 699 vertebrate messenger RNAs” Nucleic Acids Research. Vol. 15 (20) 1987, all of which are incorporated herein by reference). The protein translation initiation site (e.g. Kozak sequence) is preferably positioned immediately adjacent to the start codon.

In some embodiments the sequence encoding the 5′ UTR comprises SEQ ID NO: 438 as disclosed in International Application WO2021102107, or a functional variant thereof. In some embodiments, functional variants may have a sequence that is at least 60%, 65%, 70%, 75%, 80%, 85%, 90%, 95%, 96%, 97%, 98%, or 99% identical thereto. This 5′ UTR comprises six nucleotides of SEQ ID NO: 153 as disclosed in International Application WO2021102107, which define a Kozak sequence at the 3′ end of the CMV-IE 5′ UTR.

In some embodiments, the rAAV expressing GAA for use in the methods to treat Pompe as disclosed herein comprises an intron sequence located 3′ of the promoter sequence and 5′ of the secretory signal peptide. Intron sequences serve to increase one or more of: mRNA stability, mRNA transport out of nucleus and/or expression and/or regulation of the expressed GAA polypeptide. In alternative embodiments, a rAAV genotype does not comprise an intron sequence.

In some embodiments, the intron sequence is a MVM intron sequence, for example, but not limited to and intron sequence of SEQ ID NO: 13 as disclosed in International WO 2020102645, or nucleic acid sequence having at least 80%, 85%, 90%, 95%, 96%, 97%, 98% or 99% nucleotide sequence identity thereto.

In some embodiments, the intron sequence is a HBB2 intron sequence, for example, but not limited to and intron sequence of SEQ ID NO: 14 as disclosed in International WO 2020102645 or nucleic acid sequence having at least 80%, 85%, 90%, 95%, 96%, 97%, 98% or 99% nucleotide sequence identity thereto.

In some embodiments of the methods and compositions disclosed herein, a recombinant AAV vector comprises a heterologous nucleic acid sequence that further comprises an intron sequence located 5′ of the sequence encoding the secretory signal peptide, and 3′ of the promoter. In some embodiments, the intron sequence comprises a MVM sequence or a HBB2 sequence, wherein the MVM sequence comprises the nucleic acid sequence of SEQ ID NO: 13 as disclosed in International WO 2020102645, or a nucleic acid sequence at least about 75%, or 80%, or 85%, or 90%, or 95%, or 98%, or 99% sequence identity to SEQ ID NO: 13, and the HBB2 sequence comprises the nucleic acid sequence of SEQ ID NO: 14 as disclosed in International WO 2020102645, or a nucleic acid sequence at least about 75%, or 80%, or 85%, or 90%, or 95%, or 98%, or 99% sequence identity to SEQ ID NO: 14.

In some embodiments, the rAAV genotype comprises an intron sequence selected in the group consisting of a human beta globin b2 (or HBB2) intron, a FIX intron, a chicken beta-globin intron, and a SV40 intron. In some embodiments, the intron is optionally a modified intron such as a modified HBB2 intron (see, e.g., SEQ ID NO: 17 in of WO2018046774A1): a modified FIX intron (see., e.g., SEQ ID NO: 19 in WO2018046774A1), or a modified chicken beta-globin intron (e.g., see SEQ ID NO: 21 in WO2018046774A1), or modified HBB2 or FIX introns disclosed in WO2015/162302, which are incorporated herein in their entirety by reference.

### G. Poly-A

In some embodiments, an rAAV vector genome includes at least one poly-A tail that is located 3′ and downstream from the heterologous nucleic acid gene encoding the GAA polypeptide. In some embodiments, the polyA signal is 3′ of a stability sequence or CS sequence as defined herein. Any polyA sequence can be used, including but not limited to hGH poly A, synpA polyA and the like. In some embodiments, the polyA is a synthetic polyA sequence. In some embodiments, the rAAV vector genome comprises two poly-A tails, e.g., a hGH poly A sequence and another polyA sequence, where a spacer nucleic acid sequence is located between the two poly A sequences. In some embodiments, the rAAV genome comprises 3′ of the nucleic acid encoding the GAA polypeptide, or alternatively, 3′ of the CS sequence the following elements; a first polyA sequence, a spacer nucleic acid sequence (of between 100-400 bp, or about 250 bp), a second poly A sequence, a spacer nucleic acid sequence, and the 3′ ITR. In some embodiments, the first and second poly A sequence is a hGH poly A sequence, and in some embodiments, the first and second poly A sequences are a synthetic poly A sequence. In some embodiments, the first poly A sequence is a hGH poly A sequence and the second poly A sequence is a synthetic sequence, or vice versa—that is, in alternative embodiments, the first poly A sequence is a synthetic poly A sequence and the second poly A sequence is a hGH polyA sequence. An exemplary poly A sequence is, for example, SEQ ID NO: 15 as disclosed in International WO2021102107 (hGH poly A sequence), or a poly A nucleic acid sequence having at least 80%, 85%, 90%, 95%, 96%, 97%, 98% or 99% nucleotide sequence identity to SEQ ID NO: 15 as disclosed in International Application WO2021102107. In some embodiments, the hGHpoly sequence encompassed for use is described in Anderson et al. J. Biol. Chem 264(14); 8222-8229, 1989 (See, e.g. p. 8223, 2nd column, first paragraph) which is incorporated herein in its entirety by reference. In one embodiment, the recombinant AAV disclosed herein comprises in its genome a transcriptional terminator signal sequence or a transcriptional pause signal sequence in the reverse orientation between polyA and 3′ITR. In one embodiment, the recombinant AAV disclosed herein comprises in its genome a transcriptional terminator signal sequence or a transcriptional pause signal sequence in the 3′-5′ orientation between polyA and 3′ITR.

In some embodiments, a poly-A tail can be engineered to stabilize the RNA transcript that is transcribed from an rAAV vector genome, including a transcript for a heterologous gene, which in one embodiment is a GAA, and in alternative embodiments, the poly-A tail can be engineered to include elements that are destabilizing.

In some embodiments of the methods to treat Pompe disease as disclosed herein, a recombinant AAV vector comprises at least one polyA sequence located 3′ of the nucleic acid encoding the GAA gene and 5′ of the 3′ ITR sequence. In some embodiments, the poly A is a full length poly A (fl-polyA) sequence. In some embodiments, the polyA is a truncated polyA sequence as disclosed in International WO2021102107, which is incorporated herein in its entirety.

In an embodiment, a poly-A tail can be engineered to become a destabilizing element by altering the length of the poly-A tail. In an embodiment, the poly-A tail can be lengthened or shortened. In some embodiments, the 3′ untranslated region comprises GAA 3′ UTR (SEQ ID NO: 85) or a 3′ UTR (SEQ ID NO: 77) as disclosed in International Application WO2021102107, which is incorporated herein in its entirety by reference.

In another embodiment, a destabilizing element is a microRNA (miRNA) that has the ability to silence (repress translation and promote degradation) the RNA transcripts the miRNA binds to that encode a heterologous gene. In an embodiment, addition or deletion of seed regions within the poly-A tail can increase or decrease expression of a protein, such as the GAA protein or modified GAA polypeptide.

In another embodiment, seed regions can also be engineered into the 3′ untranslated regions located between the heterologous gene and the poly-A tail. In a further embodiment, the destabilizing agent can be an siRNA. The coding region of the siRNA can be included in an rAAV vector genome and is generally located downstream, 3′ of the poly-A tail.

In all aspects of the methods for treating Pompe disease as disclosed herein, the rAAV genome may also comprise a Stuffer DNA nucleic sequence. An exemplary stuffer DNA sequence is SEQ ID NO: 71 as disclosed in International Application WO2021102107, or a nucleic acid sequence having at least 80%, 85%, 90%, 95%, 96%, 97%, 98% or 99% nucleotide sequence identity thereto. In some embodiments, the stuffer sequence is located 3′ of the poly A tail, for example, and is located 5‘ of the’3 ITR sequence. In some embodiments, the stuffer DNA sequence comprises a synthetic polyadenylation signal in the reverse orientation.

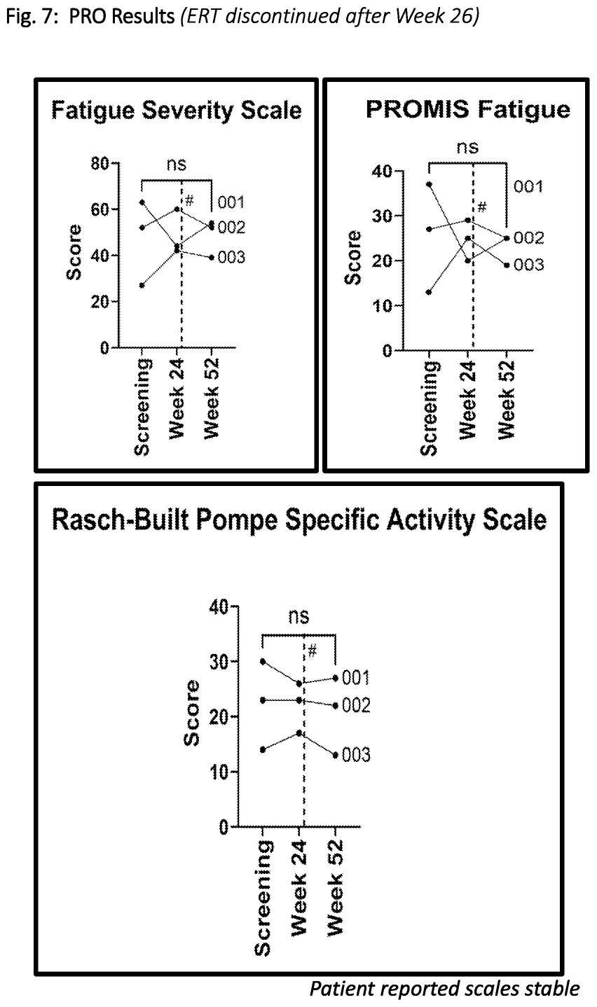

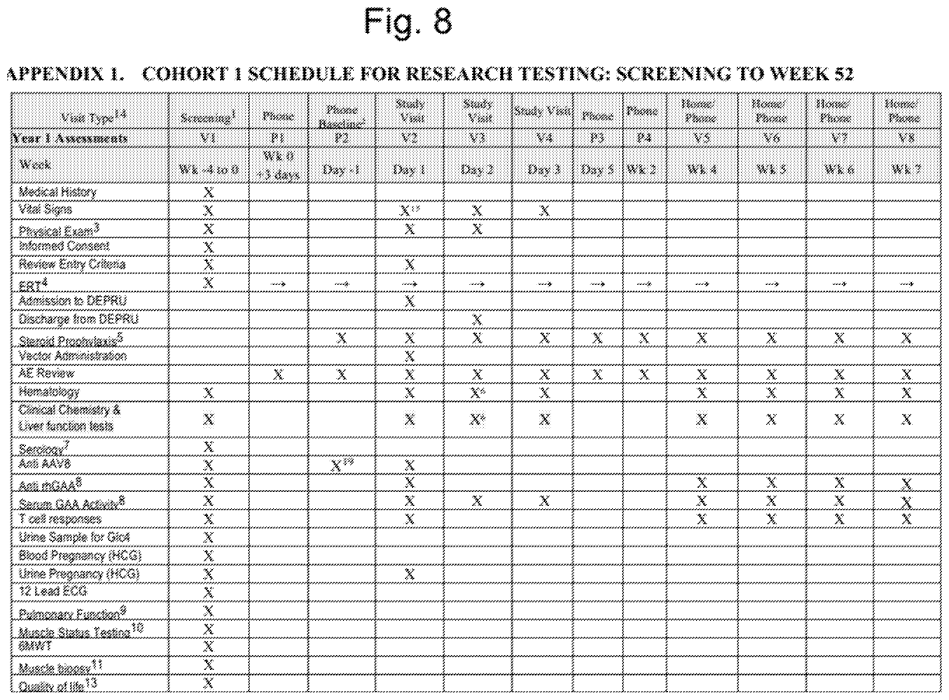

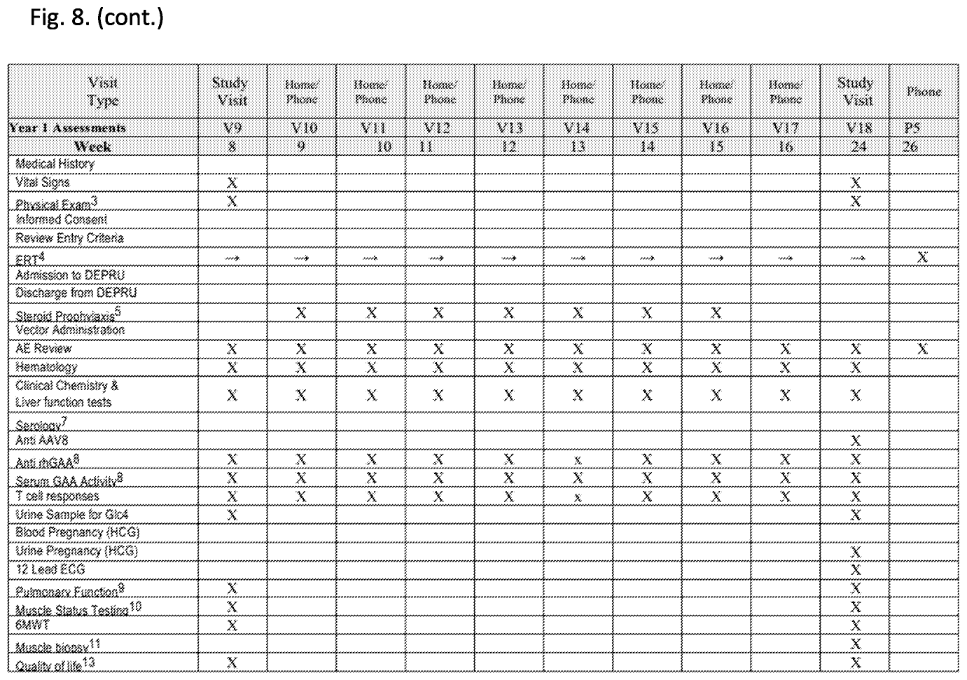

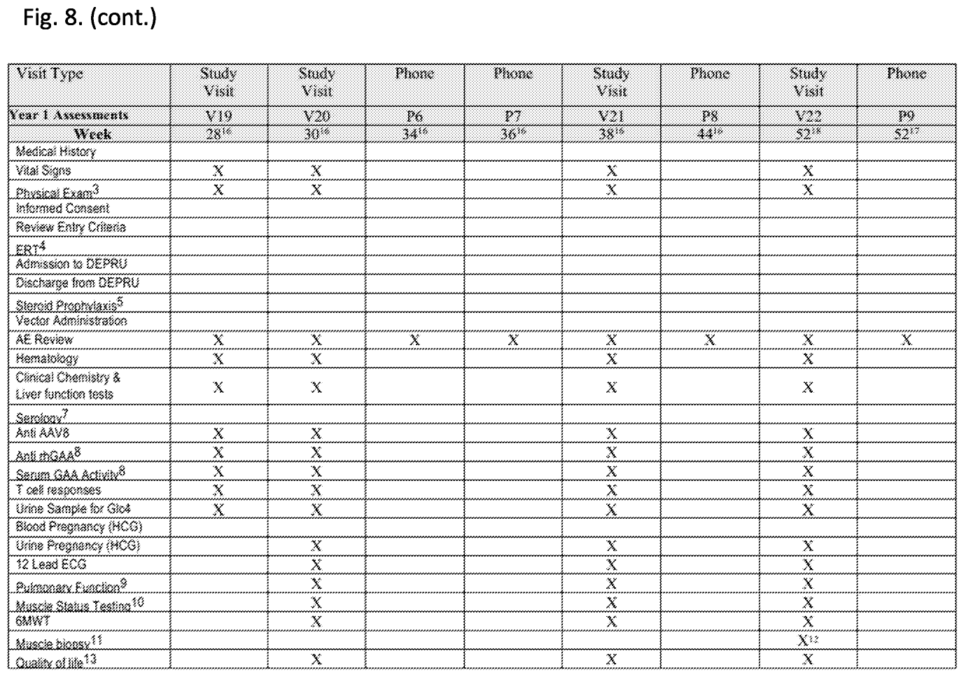

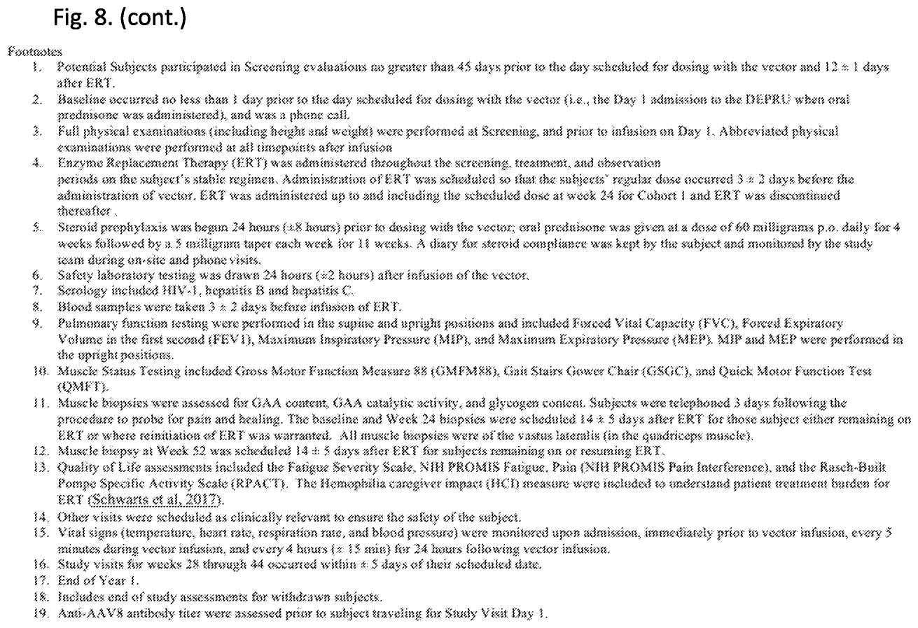

In some embodiments, a stuffer nucleic acid sequence (also referred to as a “spacer” nucleic acid fragment) can be located between the poly A sequence and the 3′ ITR (i.e., a stuffer nucleic acid sequence is located 3′ of the polyA sequence and 5′ of the 3′ ITR) (see, e.g., FIG. 7-8). Such a stuffer nucleic acid sequence can be about 30 bp, 50 pb, 75 bp, 100 bp, 150 bp, 200 bp, 250 bp, 300 bp or longer than 300 bp. In some embodiments of the methods and compositions as disclosed herein, a stuffer nucleic acid fragment is between 20-50 bp, 50-100 bp, 100-200 bp, 200-300 bp, 300-500 bp, or any integer between 20-500 bp. Exemplary stuffer (or spacer) nucleic acid sequence can be selected from any of: SEQ ID NO: 16, SEQ ID NO: 71 or SEQ ID NO: 78 as disclosed in International Application WO2021102107, or a nucleic acid sequence at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, identical to SEQ ID NO: 16 or SEQ ID NO: 71 or SEQ ID NO: 78 as disclosed in International Application WO2021102107.

### H. AAV ITRs

The rAAV vector or genome as disclosed herein for use in the methods to treat Pompe disease can comprise AAV ITRs that have desirable characteristics and can be designed to modulate the activities of, and cellular responses to vectors that incorporate the ITRs. In another embodiment, the AAV ITRs are synthetic AAV ITRs that has desirable characteristics and can be designed to manipulate the activities of and cellular responses to vectors comprising one or two synthetic ITRs, including, as set forth in U.S. Pat. No. 9,447,433, which is incorporated herein by reference.

In some embodiments, the 5′ ITR of the AAV-GAA vector of the compositions and methods described herein has the nucleotide sequence TGGGCCACTCCCTCTCTGCGCGCTCGCTCGCTCACTGAGGCCGCCCGGGCAAAGCCCGGG CGTCGGGCGACCTTTGGTCGCCCGGCCTCAGTGAGCGAGCGAGCGCGCAGAGAGGGAGT GGCCAACTCCATCACTAGGGGTTCCT (SEQ ID NO: 601). In some embodiments, the 3′ ITR of the AAV-GAA vector of the compositions and methods described herein has the nucleotide sequence AGGAACCCCTAGTGATGGAGTTGGCCACTCCCTCTCTGCGCGCTCGCTCGCTCACTGAGG CCGGGCGACCAAAGGTCGCCCGACGCCCGGGCTTTGCCCGGGCGGCCTCAGTGAGCGAG CGAGCGCGCAGAGAGGGACAGATCCG (SEQ ID NO: 602).

In another embodiment, an ITR exhibits modified transcription activity relative to a naturally occurring ITR, e.g., ITR2 from AAV2. It is known that the ITR2 sequence inherently has promoter activity. It also inherently has termination activity, similar to a poly(A) sequence. The minimal functional ITR of the present invention exhibits transcription activity as shown in the examples, although at a diminished level relative to ITR2. Thus, in some embodiments, the ITR is functional for transcription. In other embodiments, the ITR is defective for transcription. In certain embodiments, the ITR can act as a transcription insulator, e.g., preventing transcription of a transgenic cassette present in the vector when the vector is integrated into a host chromosome.

One aspect of the invention relates to an rAAV vector genome comprising at least one synthetic AAV ITR, wherein the nucleotide sequence of one or more transcription factor binding sites in the ITR is deleted and/or substituted, relative to the sequence of a naturally occurring AAV ITR such as ITR2. In some embodiments, it is the minimal functional ITR in which one or more transcription factor binding sites are deleted and/or substituted. In some embodiments at least 1 transcription factor binding site is deleted and/or substituted, e.g., at least 5 or more or 10 or more transcription factor binding sites, e.g., at least 1, 2, 3, 4, 5, 6, 7, 8, 9, 10, 11, 12, 13, 14, 15, 16, 17, 18, 19, 20, or 21 transcription factor binding sites.

Another embodiment, a rAAV vector, including an rAAV vector genome as described herein comprises a polynucleotide comprising at least one synthetic AAV ITR, wherein one or more CpG islands (a cytosine base followed immediately by a guanine base (a CpG) in which the cytosines in such arrangement tend to be methylated) that typically occur at, or near the transcription start site in an ITR are deleted and/or substituted. In an embodiment, deletion or reduction in the number of CpG islands can reduce the immunogenicity of the rAAV vector. This results from a reduction or complete inhibition in TLR-9 binding to the rAAV vector DNA sequence, which occurs at CpG islands. It is also well known that methylation of CpG motifs results in transcriptional silencing. Removal of CpG motifs in the ITR is expected to result in decreased TLR-9 recognition and/or decreased methylation and therefore decreased transgene silencing. In some embodiments, it is the minimal functional ITR in which one or more CpG islands are deleted and/or substituted. In an embodiment, AAV ITR2 is known to contain 16 CpG islands of which one or more, or all 16 can be deleted.

In some embodiments, at least 1 CpG motif is deleted and/or substituted, e.g., at least 4 or more or 8 or more CpG motifs, e.g., at least 1, 2, 3, 4, 5, 6, 7, 8, 9, 10, 11, 12, 13, 14, 15, or 16 CpG motifs.

In another embodiment, the synthetic ITR comprises, consists essentially of, or consists of one of the nucleotide sequences listed in Table 7. In other embodiments, the synthetic ITR comprises, consist essentially of, or consist of a nucleotide sequence that is at least 80% identical, e.g., at least 85%, 90%, 95%, 96%, 97%, 98%, or 99% identical to any one of the nucleotide sequences listed in Table 7. In some embodiments, the ITR is a sequence is disclosed in FIG. 1 of Samulski et al., 1983, Cell, 33; 135-143 (referred to “Samulski et al, 1983” as which is incorporated herein in its entirety by reference), which discloses modified ITR sequences in FIG. 1. In some embodiments, the ITR sequence comprises, or consists of a nucleotide sequence that is at least 80% identical, e.g., at least 85%, 90%, 95%, 96%, 97%, 98%, or 99% identical to one of the ITR sequences in FIG. 1 as disclosed in Samulski et al, 1993. In some embodiments, the ITR comprises, or consists of a nucleotide sequence that is at least 80% identical, e.g., at least 85%, 90%, 95%, 96%, 97%, 98%, or 99% or 99.5% identical to the ITR sequence of pSM 609 right disclosed in the middle panel of FIG. 1 (that lacks the 9 bp) disclosed in Samulski et al, 1983. In some embodiments, the ITR comprises a nucleotide sequence that is at least 80% identical, e.g., at least 85%, 90%, 95%, 96%, 97%, 98%, or 99% or 99.5% identical to the ITR sequence of any of SEQ ID NOs: 446-449.

In some embodiments, the ITR sequence, e.g., Right ITR (or 3′ ITR) is SEQ ID NO: 442 or a nucleotide sequence that is at least 80% identical, e.g., at least 85%, 90%, 95%, 96%, 97%, 98%, or 99% or 99.5% identical to SEQ ID NO: 442. In some embodiments, the ITR sequence, e.g., left ITR (or 5′ ITR) is SEQ ID NO: 446 or a nucleotide sequence that is at least 80% identical, e.g., at least 85%, 90%, 95%, 96%, 97%, 98%, or 99% or 99.5% identical to SEQ ID NO: 446.

TABLE 7
Exemplary synthetic ITR sequences

| | |
|---|---|
| MH-257 | AGGAACCCCTAGTGATGGAGTTGGCCACTCCCTCTCTGCGCGCTCGCTCG |
| | CTCACTGAGGCAATTTGATAAAAATCGTCAAATTATAAACAGGCTTTGCC |
| | TGTTTAGCCTCAGTGAGCGAGCGAGCGCGCAGAGAGGGAGTGGCCAACT |
| | CCATCACTAGGGGTTCCT (SEQ ID NO: 36) |
| MH-258 | AGGAACCCCTAGTGATGGAGTTGGCCACTCCCTCTCTGCGCGCTCGCTCG |
| | CTCACTGAGGGATAAAAATCCAGGCTTTGCCTGCCTCAGTGAGCGAGCG |
| | AGCGCGCAGAGAGGGAGTGGCCAACTCCATCACTAGGGGTTCCT (SEQ |
| | ID NO: 37) |
| MH Delta | AGGAACCCCTAGTGATGGAGTTGGCCACTCCCTCTCTGCGCGCTCGCTCG |
| 258 | CTCACTGAGGGATAAAAATCCAGGCTTTGCCTGCCTCAGTGAGCGAGCG |
| | AGCGCGCAGAGAGGGAGTGGCCAACTCCATCACTAGGGGTTCCT (SEQ |
| | ID NO: 38) |
| MH Telomere- | AGGAACCCCTAGTGATGGAGTTGGCCACTCCCTCTCTGGGATTGGGATTG |
| 1 ITR | CGCGCTCGCTCGCGGGATTGGGATTGGGATTGGGATTGGGATTGGGATTG |
| | ATAAAAATCAATCCCAATCCCAATCCCAATCCCAATCCCAATCCCGCGAG |
| | CGAGCGCGCAATCCCAATCCCAGAGAGGGAGTGGCCAACTCCATCACTA |
| | GGGGTTCCT (SEQ ID NO: 39) |
| MH Telomere- | AGGAACCCCTAGTGATGGAGTTGGCCACTCCCTCTCTGCGCGCTCGCTCG |
| 2 ITR | CTCGGGATTGGGATTGGGATTGGGATTGGGATTGGGATTGATAAAAATC |
| | AATCCCAATCCCAATCCCAATCCCAATCCCAATCCCGCGAGCGAGCGCGC |
| | AGGAGAGGGAGTGGCCAACTCCATCACTAGGGGTTCCTAAGCTTATTAT |
| | A (SEQ ID NO: 40) |
| MH PolII 258 | AGGAACCCCTAGTGATGGAGTTGGCCACTCCCTCTCTGCGCGCTCGCTCG |
| ITR | CTCACTGAGGGCGCCTATAAAGATAAAAATCCAGGCTTTGCCTGCCTCAG |
| | TTAGCGAGCGAGCGCGCAGAGAGGGAGTGGCCAACTCCATCACTAGGGG |
| | TTCCT (SEQ ID NO: 41) |
| MH 258 | CTAGTGATGGAGTTGGCCACTCCCTCTCTGCGCGCTCGCTCGCTCACTGA |
| Delta D | GGGATAAAAATCCAGGCTTTGCCTGCCTCAGTGAGCGAGCGAGCGCGCA |
| conservative | GAGAGGGAGTGGCCAACTCCATCACTAG (SEQ ID NO: 42) |
| 5′ ITR | TTGGCCACTCCCTCTCTGCGCGCTCGCTCGCTCACTGAGGCCGGGCGACC |
| | AAAGGTCGCCCGACGCCCGGGCTTTGCCCGGGCGGCCTCAGTGAGCGAG |
| | CGAGCGCGCAGAGAGGGAGTGGCCAACTCCATCACTAGGGGTTCCT |
| | (SEQ ID NO: 161) |
| 3′ ITR | AGGAACCCCTAGTGATGGAGTTGGCCACTCCCTCTCTGCGCGCTCGCTCG |
| | CTCACTGAGGCCGGGCGACCAAAGGTCGCCCGACGCCCGGGCTTTGCCC |
| | GGGCGGCCTCAGTGAGCGAGCGAGCGCGCAGAGAGGGAGTGGCCAA |
| | (SEQ ID NO: 165) |
| L-ITR | CTGCGCGCTCGCTCGCTCACTGAGGCCGCCCGGGCAAAGCCCGGGCGTC |
| | GGGCGACCTTTGGTCGCCCGGCCTCAGTGAGCGAGCGAGCGCGCAGAGA |
| | GGGAGTGGCCAACTCCATCACTAGGGGTTCCT (SEQ ID NO: 446) |
| R-ITR | AGGAACCCCTAGTGATGGAGTTGGCCACTCCCTCTCTGCGCGCTCGCTCG |
| | CTCACTGAGGCCGGGCGACCAAAGGTCGCCCGACGCCCGGGCTTTGCCC |
| | GGGCGGCCTCAGTGAGCGAGCGAGCGCGCAG (SEQ ID NO: 447) |
| ITR (145 bp) | AGGAACCCCTAGTGATGGAGTTGGCCACTCCCTCTCTGCGCGCTCGCTCG |
| | CTCACTGAGGCCGGGCGACCAAAGGTCGCCCGACGCCCGGGCTTTGCCC |
| | GGGCGGCCTCAGTGAGCGAGCGAGCGCGCAGAGAGGGAGTGGCCAA |
| | (SEQ ID NO: 448) |
| ITR (145 bp- | AGGAACCCCTAGTGATGGAGTTGGCCACTCCCTCTCTGCGCGCTCGCTCG |
| 1983, lacking | CTCACTGAGGCCGGGCGACCAAAGGTCGCCCGACGCCCGGGCTTTGCCC |
| 9 bp) | GGGCGGCCTCAGTGAGCGAGCGAGCGCGCAGAGAGGG (SEQ ID NO: 449) |

### IV. Vectors and Virions

In one embodiment, the rAAV vector (also referred to as a rAAV virion) as disclosed herein comprises a capsid protein, and a rAAV genome in the capsid protein. A rAAV capsid of the rAAV virion used to treat Pompe Disease is any of those listed in Table 1 disclosed in U.S. Ser. No. 62/937,556, filed on Nov. 19, 2019, (PCT/US2020/061223; WO 2021/102107) which is incorporated herein in its entirety by reference, or any combination thereof.

In one embodiment, a rAAV capsid of the rAAV virion used to treat Pompe Disease is any of those listed in Table 1 as disclosed in International Applications WO2020/102645, and WO2020/102667, each of which are incorporated herein in their entirety. In one embodiment, a rAAV capsid of the rAAV virion used to treat Pompe Disease is an AAV8 capsid. In one embodiment, a rAAV vector is an rAAV8 vector.

In one embodiment, the AAV vector (also referred to as a rAAV virion) as disclosed herein comprises a capsid protein from any of those disclosed in WO2019/241324, which is specifically incorporated herein in its entirety by reference. In some embodiments, the rAAV vector comprises a liver specific capsid, e.g., a liver specific capsid selected from XL32 and XL32.1, as disclosed in WO2019/241324, which is incorporated herein in its entirety by reference. In some embodiments, the rAAV vector is a AAVXL32 or AAVXL32.1 as disclosed in WO2019/241324, which is incorporated herein in its entirety by reference.

Exemplary chimeric or variant capsid proteins that can be used as the AAV capsid in the rAAV vector described herein can be selected from Table 2 from U.S. provisional application 62,937,556, filed on Nov. 19, 2019, which is specifically incorporated herein in its reference, or can be used with any combination with wild type capsid proteins and/or other chimeric or variant capsid proteins now known or later identified and each is incorporated herein. In some embodiments, the rAAV vector encompassed for use is a chimeric vector, e.g., as disclosed in 9,012,224 and U.S. Pat. No. 7,892,809, which are incorporated herein in their entirety by reference.

In some embodiments, the rAAV vector is a haploid rAAV vector, as disclosed in US application US2018/0371496 and PCT/US18/22725, or polyploid rAAV vector, e.g., as disclosed in PCT/US2018/044632 filed on Jul. 31, 2018 and in U.S. application Ser. No. 16/151,110, each of which are incorporated herein in their entirety by reference. In some embodiments, the rAAV vector is a rAAV3 vector, as disclosed in 9,012,224 and WO 2017/106236 which are incorporated herein in their entirety by reference.

In a particular embodiment, the rAAV is a AAVXL32 or AAVXL32.1 AAV vector as disclosed in WO2019/241324, which is incorporated herein in its entirety by reference. In some embodiments, the rAAV vector comprises a capsid disclosed in WO2019241324A1, or International Patent application PCT/US2019/036676, which are incorporated herein in their entirety by reference. In some embodiments, the AAV vector is a AAV8 vector or a rational haploid comprising an AAV8 capsid protein. In some embodiments, the recombinant AAV vector is a chimeric AAV vector, haploid AAV vector, a hybrid AAV vector or polyploid AAV vector. In some embodiments, the recombinant AAV vector is a rational haploid vector, a mosaic AAV vector, a chemically modified AAV vector, or a AAV vector from any AAV serotypes, for example, from any AAV serotype disclosed in Table 1 as disclosed in International Applications WO2020/102645, and WO2020/102667, each of which are incorporated herein in their entirety.

In an embodiment, an rAAV vector useful in the treatment of Pompe Disease as disclosed herein is an AAV3b capsid. AAV3b capsids encompassed for use are described in 2017/106236, and 9,012,224 and 7,892,809, and International application PCT/US19/61653, filed Nov. 15, 2019, and International Applications WO2020/102645, and WO2020/102667, each of which are incorporated herein in their entirety. In addition, AAV3b capsids of the AAV vector for use according to the methods as disclosed herein are disclosed in International Patent Applications WO 2020/102645 and WO2021102107, which are incorporated herein in its entirety by reference herein.

In some embodiments, the AAV3b capsid comprises SEQ ID NO: 44 as disclosed in International Patent Applications WO 2020/102645 and WO2021102107. In an embodiment, the AAV capsid used in the treatment of Pompe Disease can be a modified AAV capsid that is derived in whole or in part from the AAV capsid set forth in SEQ ID NO: 44. In some embodiments, the amino acids from an AAV3b capsid as set forth in SEQ ID NO: 44 can be, or are substituted with amino acids from another capsid of a different AAV serotype, wherein the substituted and/or inserted amino acids can be from any AAV serotype, and can include either naturally occurring or partially or completely synthetic amino acids.

In another embodiment, an AAV capsid used in the treatment of Pompe Disease is an AAV3b265D capsid. In this particular embodiment, an AAV3b265D capsid comprises a modification in the amino acid sequence of the two-fold axis loop of an AAV3b capsid via replacement of amino acid G265 of the AAV3b capsid with D265. In some embodiments, an AAV3b265D capsid comprises SEQ ID NO: 46. However, the modified virus capsids of the invention are not limited to AAV capsids set forth in SEQ ID NO: 46 as set forth in International Patent Applications WO 2020/102645 and WO2021102107. In some embodiments, the amino acids from AAV3b265D as set forth in SEQ ID NO. 46 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment an rAAV vector useful in the treatment of Pompe Disease as disclosed herein is an AAV3b265D549A capsid. In this particular embodiment, an AAV3b265D549A capsid comprises a modification in the amino acid sequence of the two-fold axis loop of an AAV3b capsid via replacement of amino acid G265 of the AAV3b capsid with D265 and replacement of amino acid T549 of the AAV3b capsid with A549. In some embodiments, an AAV3b265D549A capsid comprises SEQ ID NO: 50 as disclosed herein International Patent Applications WO 2020/102645 and WO2021102107. However, the modified virus capsids of the invention are not limited to AAV capsids set forth in SEQ ID NO: 50. In some embodiments, the amino acids from AAV3b265D549A as set forth in SEQ ID NO: 50 can be, or are substituted with amino acids from a capsid from an AAV of a different serotype, wherein the substituted and/or inserted amino acids can be from any AAV serotype, and can include either naturally occurring or partially or completely synthetic amino acids. In some embodiments, the amino acids from AAV3bSASTG (i.e., a AAV3b capsid comprising Q263A/T265 mutations) can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Pompe Disease as disclosed herein is an AAV3b549A capsid. In this particular embodiment, an AAV3b549A capsid comprises a modification in the amino acid sequence of the two-fold axis loop of an AAV3b capsid via replacement of amino acid T549 of the AAV3b capsid with A549. In some embodiments, an AAV3b549A capsid comprises SEQ ID NO: 52 as disclosed herein International Patent Applications WO 2020/102645 and WO2021102107. However, the modified virus capsids of the invention are not limited to AAV capsids set forth in SEQ ID NO: 52. In some embodiments, the amino acids from AAV3b549A as set forth in SEQ ID NO: 52 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Pompe Disease as disclosed herein is an AAV3bQ263Y capsid. In this particular embodiment, an AAV3bQ263Y capsid comprises a modification in the amino acid sequence of the two-fold axis loop of an AAV3b capsid via replacement of amino acid Q263 of the AAV3b capsid with Y263. In some embodiments, an AAV3b549A capsid comprises SEQ ID NO: 54 as disclosed herein International Patent Applications WO 2020/102645 and WO2021102107. However, the modified virus capsids of the invention are not limited to AAV capsids set forth in SEQ ID NO: 54. In some embodiments, the amino acids from AAV3bQ263Y as set forth in SEQ ID NO: 54 can be, or are substituted with amino acids from a capsid from an AAV of a different serotype, wherein the substituted and/or inserted amino acids can be from any AAV serotype, and can include either naturally occurring or partially or completely synthetic amino acids.

In another embodiment, an rAAV vector useful in the treatment of Pompe Disease as disclosed herein is AAV3bSASTG serotype or comprises a AAV3bSASTG capsid. In this particular embodiment, an AAV3bSASTG capsid comprises a modification in the amino acid sequence to comprise a SASTG mutation, in particular, the AAV3b capsid was modified to resemble AAV2 Q263A/T265 subvariant by introducing these modifications at similar positions in the AAV3b capsid (as disclosed in Messina E L, et al., Adeno-associated viral vectors based on serotype 3b use components of the fibroblast growth factor receptor signaling complex for efficient transduction. Hum. Gene Ther. 2012 October: 23(10):1031-4, Piacentino III, Valentino, et al. “X-linked inhibitor of apoptosis protein-mediated attenuation of apoptosis, using a novel cardiac-enhanced adeno-associated viral vector.” Human gene therapy 23.6 (2012): 635-646. which are both incorporated herein in their entirety by reference). Accordingly, in some embodiments, an rAAV vector useful in the treatment of Pompe Disease as disclosed herein is AAV3bSASTG serotype or comprises a AAV3bSASTG capsid comprising a AAV3b Q263A/T265 capsid. In some embodiments, the amino acids from AAV3bSASTG can be, or are substituted with amino acids from a capsid from an AAV of a different serotype, wherein the substituted and/or inserted amino acids can be from any AAV serotype, and can include either naturally occurring or partially or completely synthetic amino acids.

In order to facilitate their introduction into a cell, an rAAV vector genome useful in the invention are recombinant nucleic acid constructs that include (1) a heterologous sequence to be expressed (in one embodiment, a polynucleotide encoding a GAA polypeptide) and (2) viral sequence elements that facilitate integration and expression of the heterologous genes. The viral sequence elements may include those sequences of an AAV vector genome that are required in cis for replication and packaging (e.g., functional ITRs) of the DNA into an AAV capsid. In an embodiment, the heterologous gene encodes GAA, which is useful for correcting a GAA-deficiency in a patient suffering from Pompe Disease. In an embodiment, such an rAAV vector genome may also contain marker or reporter genes. In an embodiment, an rAAV vector genome can have one or more of the AAV3b wild-type (WT) cis genes replaced or deleted in whole or in part, but retain functional flanking ITR sequences.

### V. Optimized rAAV Vector Genome

In some embodiments of the methods and compositions as disclosed herein, an optimized rAAV vector genome is created from any of the elements disclosed herein and in any combination, including nucleic acid sequences encoding a promoter, an ITR, a poly-A tail, elements capable of increasing or decreasing expression of a heterologous gene, and in one embodiment, a nucleic acid sequence that is codon optimized for expression of GAA protein in vivo (i.e., coGAA or codon optimized GAA) and optionally, one or more element to reduce immunogenicity. Such an optimized rAAV vector genome can be used with any AAV capsid that has tropism for the tissue and cells in which the rAAV vector genome is to be transduced and expressed.

In some embodiments, rAAV genome lacks the AAV P5 promoter or, a fragment thereof, which is normally located upstream of the liver-specific promoter as disclosed herein. Normally, the P5 promoter controls expression of the AAV rep/cap proteins during AAV replication. In some embodiments, this P5 promoter fragment is present in the rAAV vector as disclosed herein which contains predicted transcription factor binding sites, e.g., cyclic AMP-responsive element-binding protein 3 (CREB3), which can be activated by endoplasmic reticulum (ER)/Golgi stress (Sampieri 2019), activating transcription factor 2 (ATF2), which is also involved in stress response (Watson 2017), Nuclear Receptor Subfamily 1 Group I Member 2 (NR1I2) (also known as Pregnane X receptor [PXR]) is known to be enriched in liver, and is activated by pregnane steroids, rifampin and other molecules including dexamethasone (NR1I2 HGNC) (Xing 2020). Accordingly, in some embodiments, a fragment of the AAV P5 promoter in the rAAV genome is removed without affecting the intended performance of the GAA cassette. In some embodiments, the rAAV vector also comprises a RNA polymerase II termination sequence located between the polyA signal and the 3′ ITR. An exemplary terminal sequence is SEQ ID NO: 450 which introduces two termination codons and one restriction site (e.g., XhoI) replaces TAG, and is located immediately downstream of the last coding amino acids of hGAA, and immediately located upstream of the 3′ UTR.

### VI. Definitions

The following terms are used in the description herein and the appended claims:

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Furthermore, the term “about,” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461,463 (CCPA 1976) (emphasis in the original); see also MPEP § 2111.03. Thus, the term “consisting essentially of” when used in a claim of this invention is not intended to be interpreted to be equivalent to “comprising.” Unless the context indicates otherwise, it is specifically intended that the various features of the invention described herein can be used in any combination.

When representing mathematical amounts, the use of E directly followed by a number should be interpreted as the same as if the number following the E were superscripted. For example, 5E11 is intended to represent the same amount as 5E”.

Moreover, the present invention also contemplates that in some embodiments of the invention, any feature or combination of features set forth herein can be excluded or omitted.

To illustrate further, if, for example, the specification indicates that a particular amino acid can be selected from A, G, I, Land/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed (e.g., by negative proviso). For example, in particular embodiments the amino acid is not A, G or I; is not A; is not G or V; etc. as if each such possible disclaimer is expressly set forth herein.

The term “parvovirus” as used herein encompasses the family Parvoviridae, including autonomously replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, Muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, volume 2, chapter 69 (4th ed., Lippincott-Raven Publishers).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of relatively new AAV serotypes and clades have been identified (see, e.g., Gao et al., (2004) J. Virology 78:6381-6388; Moris et al., (2004) Virology 33-:375-383); and also Table 1 as disclosed in U.S. Provisional Application 62,937,556, filed on Nov. 19, 2019 and Table 1 in International Applications WO2020/102645, and WO2020/102667, each of which is incorporated herein in their entirety.

The genomic sequences of various serotypes of AAV and the autonomous parvoviruses, as well as the sequences of the native inverted terminal repeats (ITRs), Rep proteins, and capsid subunits are known in the art. Such sequences may be found in the literature or in public databases such as GenBank. See, e.g., GenBank Accession Numbers NC_002077, NC_001401, NC_001729, NC_001863, NC 001829, NC 001862, NC 000883, NC_001701, NC 001510, NC_006152, NC_006261, AF063497, U89790, AF043303, AF028705, AF028704, J02275, J01901, J02275, X01457, AF288061, AH009962, AY028226, AY028223, NC 001358, NC_001540, AF513851, AF513852, AY530579; the disclosures of which are incorporated by reference herein for teaching parvovirus and AAV nucleic acid and amino acid sequences. See also, e.g., Srivistava et al., (1983) J Virology 45:555; Chiarini et al., (1998) J. Virology 71:6823; Chiarini et al., (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J Viral. 58:921; Gao et al., (2002) Proc. Nat. Acad. Sci. USA 99:11854; Morris et al., (2004) Virology 33-:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1 and Table 5 disclosed in U.S. Pat. No. 62,937,556, filed on Nov. 19, 2019 or Table 1 as disclosed in International Applications WO2020/102645, and WO2020/102667, each of which is incorporated herein in their entirety. The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99:10405-10), AAV4 (Padron et al., (2005) J. Viral. 79: 5047-58), AAV5 (Walters et al., (2004) J. Viral. 78: 3361-71) and CPV (Xie et al., (1996) J. Mal. Biol. 6:497-520 and Tsao et al., (1991) Science 251: 1456-64).

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

As used here, “systemic tropism” and “systemic transduction” (and equivalent terms) indicate that the virus capsid or virus vector of the invention exhibits tropism for and/or transduces tissues throughout the body (e.g., brain, lung, skeletal muscle, heart, liver, kidney and/or pancreas).

As used herein, “selective tropism” or “specific tropism” means delivery of virus vectors to and/or specific transduction of certain target cells and/or certain tissues.

Unless indicated otherwise, “efficient transduction” or “efficient tropism,” or similar terms, can be determined by reference to a suitable control (e.g., at least about 50%, 60%, 70%, 80%, 85%, 90%, 95%, 100%, 125%, 150%, 175%, 200%, 250%, 300%, 350%, 400%, 500% or more of the transduction or tropism, respectively, of the control). In particular embodiments, the virus vector efficiently transduces or has efficient tropism for liver cells and muscle cells. Suitable controls will depend on a variety of factors including the desired tropism and/or transduction profile.

Similarly, it can be determined if a virus “does not efficiently transduce” or “does not have efficient tropism” for a target tissue, or similar terms, by reference to a suitable control. In particular embodiments, the virus vector does not efficiently transduce (i.e., has does not have efficient tropism) for kidney, gonads and/or germ cells. In particular embodiments, transduction (e.g., undesirable transduction) of tissue(s) (e.g., kidney) is 20% or less, 10% or less, 5% or less, 1% or less, 0.1% or less of the level of transduction of the desired target tissue(s) (e.g., liver, skeletal muscle, diaphragm muscle, cardiac muscle and/or cells of the central nervous system).

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotides), but in representative embodiments are either single or double stranded DNA sequences.

The terms “heterologous nucleotide sequence” and “heterologous nucleic acid molecule” are used interchangeably herein and refer to a nucleic acid sequence that is not naturally occurring in the virus. Generally, the heterologous nucleic acid molecule or heterologous nucleotide sequence comprises an open reading frame that encodes a polypeptide and/or nontranslated RNA of interest (e.g., for delivery to a cell and/or subject).

A “chimeric nucleic acid” comprises two or more nucleic acid sequences covalently linked together to encode a fusion polypeptide. The nucleic acids may be DNA, RNA, or a hybrid thereof.

The term “fusion polypeptide” comprises two or more polypeptides covalently linked together, typically by peptide bonding.

As used herein, an “isolated” polynucleotide (e.g., an “isolated DNA” or an “isolated RNA”) means a polynucleotide at least partially separated from at least some of the other components of the naturally occurring organism or virus, for example; the cell or viral structural components or other polypeptides or nucleic acids commonly found associated with the polynucleotide. In representative embodiments an “isolated” nucleotide is enriched by at least about 10-fold, 100′-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

A population of virions can be generated by any of the methods described herein. In one embodiment, the population is at least 10¹ virions. In one embodiment, the population is at least 10² virions, at least 10³, virions, at least 10⁴ virions, at least 10⁵ virions, at least 10⁶ virions, at least 10⁷ virions, at least 10⁸ virions, at least 10⁹ virions, at least 10¹⁰ virions, at least 10¹¹ virions, at least 10¹² virions, at least 10¹³ virions, at least 10¹⁴ virions, at least 10¹⁵ virions, at least 10¹⁶ virions, or at least 10¹⁷ virions. A population of virions can be heterogeneous or can be homogeneous (e.g., substantially homogeneous or completely homogeneous).

A “substantially homogeneous population” as the term is used herein, refers to a population of virions that are mostly identical, with few to no contaminant virions (those that are not identical) therein. A substantially homogeneous population is at least 90% of identical virions (e.g., the desired virion), and can be at least 91%, at least 92%, at least 93%, at least 94%, at least 95%, at least 96%, at least 97%, at least 98%, at least 99%, at least 99.5%, at least 99.9% of identical virions.

A population of virions that is completely homogeneous contains only identical virions.

As used herein, by “isolate” or “purify” (or grammatical equivalents) a virus vector or virus particle or population of virus particles, it is meant that the virus vector or virus particle or population of virus particles is at least partially separated from at least some of the other components in the starting material. In representative embodiments an “isolated” or “purified” virus vector or virus particle or population of virus particles is enriched by at least about 10-fold, 100-fold, 1000-fold, 10,000-fold or more as compared with the starting material.

Unless indicated otherwise, “efficient transduction” or “efficient tropism,” or similar terms, can be determined by reference to a suitable control (e.g., at least about 10%, 15%, 20%, 25%, 30%, 35%, 40%, 45%, 50%, 55%, 60%, 65%, 70%, 75%, 80%, 85%, 90%, 95%, 100%, 125%, 150%, 175%, 200%, 250%, 300%, 350%, 400%, 500% or more of the transduction or tropism, respectively, of the control). In particular embodiments, the virus vector efficiently transduces or has efficient tropism for neuronal cells and cardiomyocytes. Suitable controls will depend on a variety of factors including the desired tropism and/or transduction profile.

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., enzyme replacement to reduce or eliminate symptoms of a disease, or improvement in transplant survivability or induction of an immune response.

The terms “heterologous nucleotide sequence” and “heterologous nucleic acid molecule” are used interchangeably herein and refer to a nucleic acid sequence that is not naturally occurring in the virus. Generally, the heterologous nucleic acid molecule or heterologous nucleotide sequence comprises an open reading frame that encodes a polypeptide and/or nontranslated RNA of interest (e.g., for delivery to a cell and/or subject), for example the GAA polypeptide.

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

An “rAAV vector genome” or “rAAV genome” is an AAV genome (i.e., vDNA) that comprises one or more heterologous nucleic acid sequences. rAAV vectors generally require only the inverted terminal repeat(s) (TR(s)) in cis to generate virus. All other viral sequences are dispensable and may be supplied in trans (Muzyczka, (1992) Curr. Topics Microbial. Immunol. 158:97). Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV TR sequence), optionally two ITRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., an ITR that mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR,” including an “AAV inverted terminal repeat” or “AAV ITR” may be from any AAV, including but not limited to serotypes 1, 2, 3, 4, 5, 6, 7, 8, 9, 10, 11 or 12 or any other AAV now known or later discovered. An AAV terminal repeat need not have the native terminal repeat sequence (e.g., a native AAV TR or AAV ITR sequence may be altered by insertion, deletion, truncation and/or missense mutations), as long as the terminal repeat mediates the desired functions, e.g., replication, virus packaging, integration, and/or provirus rescue, and the like.

AAV proteins VP1, VP2 and VP3 are capsid proteins that interact together to form an AAV capsid of an icosahedral symmetry. VP1.5 is an AAV capsid protein described in US Publication No. 2014/0037585.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

The recombinant AAV vector expressing human GAA can be produced by the triple transfection method that uses close ended linear duplexed DNA molecules that lack bacterial backbone sequences, for example, as described in PCT/US2021/013689, published as WO/2021/146591, which is incorporated herein by reference in its entirety.

In several embodiments of the invention the rAAV comprising SEQ ID NO: 606 that encodes hGAA (as set forth in SEQ ID NO: 600) is manufactured using plasmid DNA as starting material. In some embodiments of the invention, rAAV comprising SEQ ID NO: 606 that encodes hGAA (as set forth in SEQ ID NO: 600) is manufactured using close ended linear duplexed DNA as starting material.

Further, the viral capsid or genomic elements can contain other modifications, including insertions, deletions and/or substitutions.

A “chimeric’ capsid protein as used herein means an AAV capsid protein (e.g., any one or more of VP1, VP2 or VP3) that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the literature as well as herein that can be included in the capsid of this invention.

As used herein, the term “haploid AAV” shall mean that AAV as described in International Application WO2018/170310, or US Application US2018/037149, which are incorporated herein in their entirety by reference. In some embodiments, a population of virions is a haploid AAV population where a virion particle can be constructed wherein at least one viral protein from the group consisting of AAV capsid proteins, VP1, VP2 and VP3, is different from at least one of the other viral proteins, required to form the virion particle capable of encapsulating an AAV genome. For each viral protein present (VP1, VP2, and/or VP3), that protein is the same type (e.g., all AAV2 VP1). In one instance, at least one of the viral proteins is a chimeric viral protein and at least one of the other two viral proteins is not a chimeric. In one embodiment VP1 and VP2 are chimeric and only VP3 is non-chimeric. For example, only the viral particle composed of VP1/VP2 from the chimeric AAV2/8 (the N-terminus of AAV2 and the C-terminus of AAV8) paired with only VP3 from AAV2; or only the chimeric VP1/VP2 28m-2P3 (the N-terminal from AAV8 and the C-terminal from AAV2 without mutation of VP3 start codon) paired with only VP3 from AAV2. In another embodiment only VP3 is chimeric and VP1 and VP2 are non-chimeric. In another embodiment at least one of the viral proteins is from a completely different serotype. For example, only the chimeric VP1/VP2 28m-2P3 paired with VP3 from only AAV3. In another example, no chimeric is present.

The term a “hybrid” AAV vector or parvovirus refers to a rAAV vector where the viral TRs or ITRs and viral capsid are from different parvoviruses. Hybrid vectors are described in international patent publication WO 00/28004 and Chao et al., (2000) Molecular Therapy 2:619. For example, a hybrid AAV vector typically comprises the adenovirus 5′ and 3′ cis ITR sequences sufficient for adenovirus replication and packaging (i.e., the adenovirus terminal repeats and PAC sequence).

The term “polyploid AAV” refers to a AAV vector which is composed of capsids from two or more AAV serotypes, e.g., and can take advantages from individual serotypes for higher transduction but not in certain embodiments eliminate the tropism from the parents.

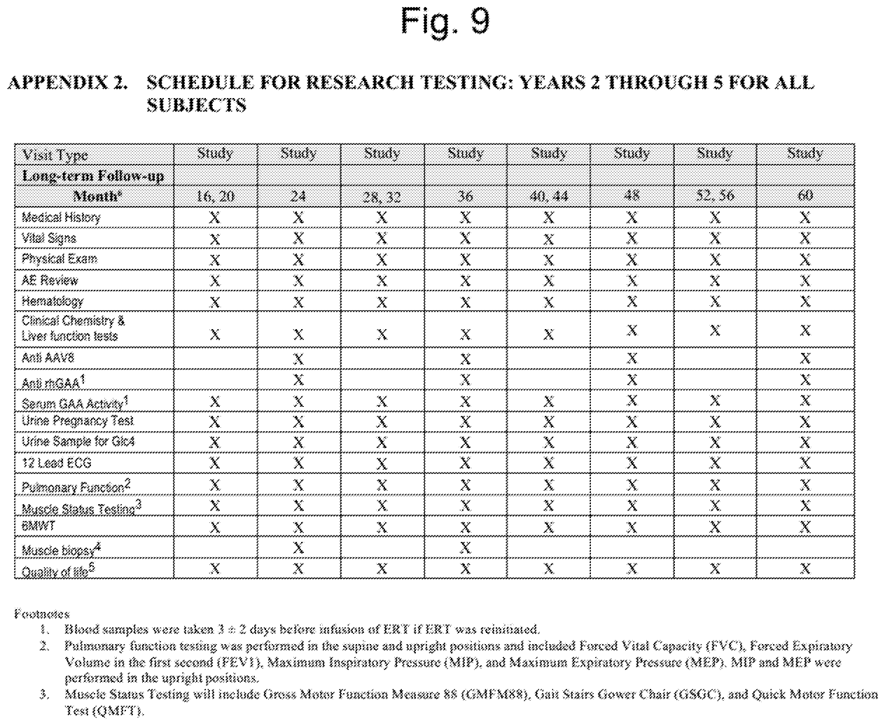

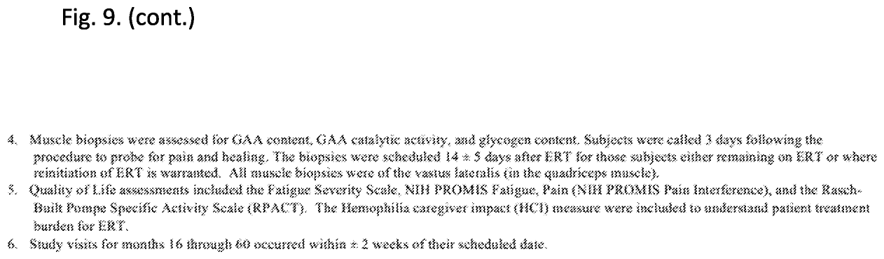

The term “GAA” or “GAA polypeptide,” as used herein, encompasses mature (^{˜}76 or ^{˜}67 kDa) and precursor (e.g., ^{˜}110 kDa) GAA as well as modified (e.g., truncated or mutated by insertion(s), deletion(s) and/or substitution(s)) GAA proteins or fragments thereof that retain biological function (i.e., have at least one biological activity of the native GAA protein, e.g., can hydrolyze glycogen, as defined above) and GAA variants (e.g., GAA II as described by Kunita et al., (1997) Biochemica et Biophysica Acta 1362:269; GAA polymorphisms and SNPs are described by Hirschhorn, R. and Reuser, A. J. (2001) in The Metabolic and Molecular Basis for Inherited Disease (Scriver, C. R., Beaudet. A. L., Sly, W. S. & Valle, D. Eds.), pp. 3389-3419, McGraw-Hill, New York, see pages 3403-3405; each incorporated herein by reference in its entirety). Any GAA coding sequence known in the art may be used, for example, see the coding sequences of FIGS. 8 and 9; GenBank Accession number NM_00152 and Hoefsloot et al., (1988) EMBO J. 7:1697 and Van Hove et al., (1996) Proc. Natl. Acad. Sci. USA 93:65 (human), GenBank Accession number NM_008064 (mouse), and Kunita et al., (1997) Biochemica et Biophysics Acta 1362:269 (quail); the disclosures of which are incorporated herein by reference for their teachings of GAA coding and noncoding sequences. The term “GAA”, or, “hGAA” or, “rhGAA” is used interchangeably to refer to nucleic acid encoding human GAA or, to human GAA polypeptide.

The term “targeting peptide” is also referred to as a “targeting sequence” as used herein is intended to refer to a peptide that targets a particular subcellular compartment, for example, a mammalian lysosome. A targeting peptide encompassed for use herein is a lysosome targeting peptide that is mannose-6-phosphate-independent. An exemplary targeting sequence is an IGF2 targeting peptide as disclosed herein.

The term “signal sequence” is used interchangeably herein with the term “secretory signal sequence” or “leader sequence” or “signal peptide” or variations thereof, and intended to refer to amino acid sequences that function to enhance (as defined above) secretion of an operably linked polypeptide, (e.g., a GAA peptide) from the cell as compared with the level of secretion seen with the native polypeptide. As defined above, by “enhanced” secretion, it is meant that the relative proportion of GAA polypeptide synthesized by the cell that is secreted from the cell is increased; it is not necessary that the absolute amount of secreted protein is also increased. In particular embodiments of the invention, essentially all (i.e., at least 95%, 97%, 98%, 99% or more) of the GAA-polypeptide is secreted. It is not necessary, however, that essentially all or even most of the GAA polypeptide is secreted, as long as the level of secretion is enhanced as compared with the native GAA polypeptide. Exemplary leader sequences include, but are not limited to the innate GAA signal sequence (also referred to cognate GAA leader sequence or endogenous GAA signal sequence), AAT sequence, IL2(1-3), IL2 leader sequence (IL2 wt), a modified IL2 leader sequence (IL2 mut), fibronectin (FN1, also referred to as FBN), or IgG leader sequence or functional variants thereof, as disclosed herein.

As used herein, the term “amino acid” encompasses any naturally occurring amino acid, modified forms thereof, and synthetic amino acids. Naturally occurring, levorotatory (L-) amino acids are disclosed in Table 2 of US Publication 2018/0371496, which is incorporated herein in its entirety. Alternatively, the amino acid can be a modified amino acid residue (nonlimiting examples are shown in Table 4 of US Publication of US Publication 2018/0371496) and/or can be an amino acid that is modified by post-translation modification (e.g., acetylation, amidation, formylation, hydroxylation, methylation, phosphorylation or sulfatation). Further, the non-naturally occurring amino acid can be an “unnatural” amino acid as described by Wang et al., Annu Rev Biophys Biomol Struct. 35:225-49 (2006). These unnatural amino acids can advantageously be used to chemically link molecules of interest to the AAV capsid protein.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed (e.g., by negative proviso). For example, in particular embodiments the amino acid is not A, G or I; is not A; is not G or V; etc. as if each such possible disclaimer is expressly set forth herein.

As used herein, the phrase “promoter” refers to a region of DNA that generally is located upstream of a nucleic acid sequence to be transcribed that is needed for transcription to occur, i.e. which initiates transcription. Promoters permit the proper activation or repression of transcription of a coding sequence under their control. A promoter typically contains specific sequences that are recognized and bound by plurality of TFs. TFs bind to the promoter sequences and result in the recruitment of RNA polymerase, an enzyme that synthesizes RNA from the coding region of the gene. A great many promoters are known in the art.

The term “synthetic promoter” as used herein relates to a promoter that does not occur in nature. Parts of the synthetic promoter may be naturally occurring (e.g. the minimal promoter), but the synthetic promoter as a complete entity is not naturally occurring.

As used herein, “minimal promoter” (also known as the “core promoter”) refers to a short DNA segment which is inactive or largely inactive by itself, but can mediate transcription when combined with other transcription regulatory elements. Minimum promoter sequence can be derived from various different sources, including prokaryotic and eukaryotic genes. Examples of minimal promoters are discussed above, and include the dopamine beta-hydroxylase gene minimum promoter, cytomegalovirus (CMV) immediate early gene minimum promoter (CMV-MP), and the herpes thymidine kinase minimal promoter (MinTK). A minimal promoter typically comprises the transcription start site (TSS) and elements directly upstream, a binding site for RNA polymerase II, and general transcription factor binding sites (often a TATA box).

As used herein, “proximal promoter” relates to the minimal promoter plus the proximal sequence upstream of the gene that tends to contain primary regulatory elements. It often extends approximately 250 base pairs upstream of the TSS, and includes specific TFBS. The proximal promoter can be a naturally occurring liver-specific proximal promoter. However, the proximal promoter can be synthetic.

A “functional variant” of a promoter or other nucleic acid sequence in the context of the present invention is a variant of a reference sequence that retains the ability to function in the same way as the reference sequence, e.g. as a liver-specific promoter. Alternative terms for such functional variants include “biological equivalents” or “equivalents”.

The terms “liver-specific” or “liver-specific expression” when in reference to a promoter refers to the ability of promoter to enhance or drive expression of a gene in the liver (or in liver-derived cells) in a preferential or predominant manner as compared to other tissues (e.g. spleen, muscle, heart, lung, and brain). Expression of the gene can be in the form of mRNA or protein. In some embodiments, liver-specific expression is such that there is negligible expression in other (i.e. non-liver) tissues or cells, i.e. expression is highly liver-specific. In some embodiments, while a liver-specific promoter drives expression preferentially in the liver, it can also drive expression of the gene in another tissue of interest at a lower level, e.g., muscle.

The skilled person can thus easily determine whether any variant of the liver-specific promoter recited above remains functional (i.e. it is a functional variant as defined above). For example, any given promoter to be assessed can be operably linked to a minimal promoter (e.g. positioned upstream of CMV-MP) and the ability of the promoter to drive liver-specific expression of a gene (typically a reporter gene) is measured. Similarly, the ability of a promoter to drive liver-specific expression can be readily assessed by the skilled person (e.g. as described in the examples below). Expression levels of a gene driven by a variant of a reference promoter can be compared to the expression levels driven by the reference sequence. In some embodiments, where liver-specific expression levels driven by a variant promoter are at least 50%, at least 60%, at least 70%, at least 80%, at least 90%, or at least 100% of the expression levels driven by the reference promoter, it can be said that the variant remains functional. Suitable nucleic acid constructs and reporter assays to assess liver-specific expression enhancement can easily be constructed, and the examples set out below give suitable methodologies.

Liver-specificity can be identified wherein the expression of a gene (e.g. a therapeutic or reporter gene) occurs preferentially or predominantly in liver-derived cells. Preferential or predominant expression can be defined, for example, where the level of expression is significantly greater in liver-derived cells than in other types of cells (i.e. non-liver-derived cells). For example, expression in liver-derived cells is suitably at least 5-fold higher than non-liver cells, preferably at least 10-fold higher than non-liver cells, and it may be 50-fold higher or more in some cases. For convenience, liver-specific expression can suitably be demonstrated via a comparison of expression levels in a hepatic cell line (e.g. liver-derived cell line such as Huh7 and/or HepG2 cells) or liver primary cells, compared with expression levels in a kidney-derived cell line (e.g. HEK-293), a cervical tissue-derived cell line (e.g. HeLa) and/or a lung-derived cell line (e.g. A549).

The synthetic liver-specific promoters of the present invention are preferably suitable for promoting expression in the liver of a subject, e.g., driving liver-specific expression of a transgene, preferably a therapeutic transgene.

Preferred synthetic liver-specific promoters of the present invention are suitable for promoting liver-specific transgene expression and have an activity in liver cells which is at least 15%, 20%, 30%, 40%, 50%, 60%, 70%, 80%, 90%, 100%, 125%, 150%, 175%, 200%, 250%, 300%, 350% or 400% of the activity of the TBG promoter (see, e.g., SEQ ID NO: 435 as disclosed in International Application WO2021102107).

The synthetic liver-specific promoters of the present invention are preferably suitable for promoting liver-specific expression at a level at least 1.5-fold greater than a CMV-IE promoter (see, e.g., SEQ ID NO: 433 as disclosed in International Application WO2021102107) in liver-derived cells, preferably at least 2-fold greater than a CMV promoter in liver-derived cells (e.g. HEK-293, HeLa, and/or A549 cells).

The terms “identity” and “identical” and the like refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

The term “synthetic” as used herein means a nucleic acid molecule that does not occur in nature. Synthetic nucleic acid expression constructs of the present invention are produced artificially, typically by recombinant technologies. Such synthetic nucleic acids may contain naturally occurring sequences (e.g. promoter, enhancer, intron, and other such regulatory sequences), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof.

A “spacer sequence” or “spacer” as used herein is a nucleic acid sequence that separates two functional nucleic acid sequences. It can have essentially any sequence, provided it does not prevent the functional nucleic acid sequence (e.g. cis-regulatory element) from functioning as desired (e.g. this could happen if it includes a silencer sequence, prevents binding of the desired transcription factor, or suchlike). Typically, it is non-functional, as in it is present only to space adjacent functional nucleic acid sequences from one another.

The term “pharmaceutically acceptable” as used herein is consistent with the art and means compatible with the other ingredients of the pharmaceutical composition and not deleterious to the recipient thereof.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is substantially less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

The phrase a “therapeutically effective amount” and like phrases mean a dose or plasma concentration in a subject that provides the desired specific pharmacological effect, e.g. to express a therapeutic gene in the liver, and secretion into the plasma. It is emphasized that a therapeutically effective amount may not always be effective in treating the conditions described herein, even though such dosage is deemed to be a therapeutically effective amount by those of skill in the art. The therapeutically effective amount may vary based on the route of administration and dosage form, the age and weight of the subject, and/or the disease or condition being treated.

The terms “individual,” “subject,” and “patient” are used interchangeably, and refer to any individual subject with a disease or condition in need of treatment. For the purposes of the present disclosure, the subject may be a primate, preferably a human, or another mammal, such as a dog, cat, horse, pig, goat, or bovine, and the like.

Additional patents incorporated for reference herein that are related to, disclose or describe an AAV or an aspect of an AAV, including the DNA vector that includes the gene of interest to be expressed are: U.S. Pat. Nos. 6,491,907; 7,229,823; 7,790,154; 7,201,898; 7,071,172; 7,892,809; 7,867,484; 8,889,641; 9,169,494; 9,169,492; 9,441,206; 9,409,953; and, 9,447,433; 9,592,247; and, 9,737,618.

The present invention may be as defined in any one of the following numbered paragraphs.

1. A method of treating Pompe disease in a subject, comprising administering to a subject who is being treated for Pompe disease with long-term GAA enzyme replacement therapy (ERT), a pharmaceutical composition comprising a recombinant adeno-associated virus (AAV) vector comprising in its genome, a heterologous nucleic acid sequence encoding a polypeptide comprising an alpha-glucosidase (GAA) polypeptide in expressible form at a dosage no more than 4.0E¹² vg/kg, wherein the heterologous nucleic acid is operatively linked to a liver-specific promoter, and wherein the administration of long-term GAA enzyme replacement therapy (ERT) is withdrawn by at least 26 weeks post administration of the recombinant AAV, and wherein the subject obtains a blood serum level of GAA expressed by the AAV at a pharmacological activity is at least 165 nmol/ml/hr of at least within two weeks of administration.
2. The method of paragraph 1, wherein the Pompe disease is late onset Pompe disease (LOPD) or infantile-onset Pompe disease (IOPD).
3. The method of paragraph 1 or 2, wherein the subject has previously undergone long-term administration of ERT for Pompe disease.
4. The method of paragraph 3, wherein the administration of ERT in the subject is withdrawn concurrently or prior to the administration of the AAV vector.
5. The method of paragraph 4, wherein the administration of ERT is withdrawn on the same day (d1), or at least the day before the administration of the AAV vector.
6. The method of paragraph 3, wherein the administration of ERT is withdrawn after the administration of the AAV vector.
7. The method of paragraph 6, wherein the administration of ERT is withdrawn during a period of about day 0 of the administration to about 26 weeks after the administration of the AAV vector.
8. The method of paragraph 6, wherein the administration of ERT is withdrawn at about 24 weeks or about 26 weeks following the administration of the AAV vector.
9. The method of any one of paragraphs 1-8, further comprising administering complementary ERT after an extended period of time following the administration of the AAV vector and further following withdrawal of the administration of the long term ERT.
10. The method of paragraph 9, wherein the extended period of time is about 25 weeks, about 97 weeks, about 107 weeks after the administration of the AAV vector.
11. The method of paragraph 9, wherein the extended period of time is at least 1 year following the administration of the AAV vector.
12. The method of paragraph 9, wherein the extended period of time is between 12-18 months after the administration of the AAV vector.
13. The method of paragraph 9, wherein the extended period of time is at least 6-months after the administration of the AAV vector.
14. The method of any one of paragraphs 9-13, wherein the complementary ERT is administered at a lower frequency and/or dosage than the administration of the long-term ERT.
15. The method of paragraph 14, wherein the complementary ERT is administered at regular intervals.
16. The method of paragraph 15, wherein the complementary ERT is suspended for an extended period of time of at least 10 weeks.
17. The method of paragraph 14, wherein the complementary ERT administration is sporadic.
18. The method of any one of the above paragraphs, wherein the pharmaceutical composition comprises a dose of rAAV of between 1.6E12 vg/kg and 3.2E12 vg/kg.
19. The method of paragraph 17, wherein the dose of rAAV is sufficient to express GAA to achieve clinical stability in the subject in one or more symptoms of Pompe disease, wherein clinical stability is selected from the group consisting of a) not exceeding a 15% decrease in Forced Vital Capacity (FVC) over two consecutive assessments, measured no less than 3-months apart, b) not exceeding a 12% decrease in the 6MWT over two consecutive assessment, measured no less than 3-months apart, and c) not exceeding a 43-meter decrease in the 6MWT over two consecutive assessments, measured no less than 3-months apart.
20. The method of any one of the above paragraphs, wherein the pharmaceutical composition comprises a dose of rAAV sufficient to achieve serum level of hGAA expressed by the rAAV at a pharmacological activity range from at least about 165 to ≤2,260 nmol/ml/hr.
21. The method of any one of the above paragraphs, wherein the heterologous nucleic acid sequence encoding the GAA polypeptide further comprises a nucleic acid encoding a secretory signal peptide located at the 5′ of the nucleic acid encoding the GAA polypeptide.
22. The method of paragraph 21, wherein the signal sequence is an endogenous GAA signal sequence fused to the 5′ end of the nucleic acid encoding the GAA polypeptide.
23. The method of paragraph 21, wherein the signal sequence is a heterologous signal sequence fused to the 5′ end of the nucleic acid encoding the GAA polypeptide.
24. The method of paragraph 23, wherein the heterologous signal sequence is selected from any of: fibronectin (FN1) IL2WT, 201IgG, IL2mut, AAT, preprocathepsin L, pre-pro-alpha 2 type collagen.
25. The method of any one of paragraphs 1-24, wherein the nucleic acid sequence encodes a wild-type GAA polypeptide or a modified GAA polypeptide.
26. The method of any one of paragraphs 1-25, wherein the nucleic acid sequence encoding the GAA polypeptide is the human GAA gene or a human codon optimized GAA gene (coGAA) or a modified GAA nucleic acid sequence.
27. The method of any one of paragraphs 1-26, wherein the nucleic acid sequence encoding the GAA polypeptide encodes a GAA polypeptide which comprises at least one, at least 2 or at least all three amino acid modifications selected from H201L, H199R, R233H, V780I or V780R of SEQ ID NO: 10.
28. The method of any one of paragraphs 1-22, wherein the heterologous nucleic acid sequence encodes a GAA polypeptide having the amino acid sequence of SEQ ID NO: 600 or a functional variant or functional fragment thereof having at least 90%, 95% or 99% activity to SEQ ID NO: 600.
29. The method of any one of paragraphs 1-28, wherein the heterologous nucleic acid sequence encoding a GAA polypeptide is located between a 5′ ITR and a 3′ ITR sequence.
30. The method of any one of paragraphs 1-29, wherein the AAV vector further comprises in its genome at least one polyA sequence located between 3′ end of the nucleic acid encoding the GAA gene and 5′ end of the 3′ ITR sequence.
31. The method of any one of paragraphs 29-30, wherein the ITR comprises an insertion, deletion or substitution.
32. The method of paragraph 31, wherein one or more CpG islands in the ITR are removed.
33. The method of any one of paragraphs 1-32, wherein the recombinant AAV vector is a chimeric AAV vector, haploid AAV vector, a hybrid AAV vector or polyploid AAV vector.
34. The method of any one of paragraphs 1-33, wherein the recombinant AAV vector is a rational haploid vector, a mosaic AAV vector, a chemically modified AAV vector, or a AAV vector from any AAV serotypes.
35. The method of any one of paragraphs 1-34, wherein the recombinant AAV vector is selected from the group consisting of: AAV3b, a AAVXL32 vector, a AAVXL32.1 vector, a AAV8 vector, or a haploid AAV8 vector comprising at least one AAV8 capsid protein.
36. The method of paragraph 35, wherein the AAV3b serotype comprises one or more mutations in a capsid protein selected from any of: 265D, 549A, Q263Y.
37. The method of paragraph 36, wherein the AAV3b serotype is selected from any of: AAV3b265D, AAV3b265D549A, AAV3b549A or AAV3bQ263Y, or AAV3bSASTG.
38. The method of any one of paragraphs 1-35, wherein the recombinant AAV vector is serotype AAV8, or comprises at least one AAV8 capsid protein.
39. The method of any one of paragraphs 1-36, further comprising administering to the subject an immunosuppressant for a defined period of time.
40. The method of paragraph 39, wherein the subject is administered an immunosuppressant at a first dose for a first time period, the first time period beginning the day before, or on the day of the administration of the AAV vector.
41. The method of paragraph 39, and wherein the subject is administered an immunosuppressant at incremental tapering doses, each incremental tapering dose is administered for a defined period of time after the first time period of administration of the immunosuppressant at the first dose.
42. The method of paragraph 41, and wherein the subject is administered an immunosuppressant at a first dose for 4 weeks, and administered each incremental tapering dose for 1 week thereafter.
43. The method of any one of paragraphs 1-38, wherein the subject is not administered an immunosuppressant.
44. The method of any one of paragraphs 1-43, wherein the pharmaceutical composition further comprises a pharmaceutically acceptable carrier, but lacks a GAA ERT.
45. The method of any one of paragraphs 1-44, wherein the GAA polypeptide is secreted from the subject's liver to thereby result in uptake of the secreted GAA by skeletal muscle tissue, cardiac muscle tissue, diaphragm muscle tissue or a combination thereof, that results in a reduction in lysosomal glycogen stores in the tissue(s).
46. The method of paragraph 45, wherein the reduction in lysosomal glycogen stores in the tissue(s) results in reduced symptoms of Pompe disease and/or clinically stable reduction in lysosomal glycogen stores in the subject.
47. The method of any one of paragraphs 1-46, wherein administration is selected from the group consisting of: intramuscular, sub-cutaneous, intraspinal, intracisternal, intrathecal, intravenous, and combinations thereof.
48. The method of any one of paragraphs 1-47, wherein T cell reactivity to vector capsid is substantially low.
49. The method of paragraph 48, wherein T cell reactivity is below a threshold range of >40 to <129/sfu/million.
50. A method of reducing or eliminating the clinical need for GAA enzyme replacement therapy (ERT) in a subject with Pompe disease, comprising or consisting essentially of, administering to the subject a pharmaceutical composition comprising a recombinant adeno-associated virus (AAV) vector comprising in its genome, a heterologous nucleic acid sequence encoding a polypeptide comprising an alpha-glucosidase (GAA) polypeptide in expressible form at a dosage no more than 4.0E¹² vg/kg, wherein the heterologous nucleic acid is operatively linked to a liver-specific promoter, and wherein the subject obtains a blood serum level of GAA expressed by the AAV at a pharmacological activity range from 165 to ≤2,260 nmol/ml/hr of at least within two weeks of administration.
51. The method of paragraph 50, wherein the frequency of administration of the GAA ERT and/or dosage of GAA ERT administered is reduced by at least 20% in the first year after administration of the AAV vector as compared to the year prior to said administration.
52. The method of paragraphs 50 or 51, wherein the level and/or rate of clinical decline of the subject over time upon cessation of ERT is reduced by at least 50%.
53. The method of any of paragraphs 50-52, wherein ERT can be withdrawn for an extended period of time without substantial clinical decline.
54. The method of paragraph 53, wherein the extended period of time is selected from the group consisting of 2-3 weeks, 1-3 months, 3-6 months, 6-12 months, 12 months-1.5 years, and 1 year-2 years.
55. The method of any of the preceeding paragraphs, wherein GAA activity in muscle is increased by at least 2 fold than the level before the AAV vector was administered.
56. The method of any of the preceeding paragraphs, wherein GAA activity in muscle is increased by at least 2 fold than the level after the long term ERT was withdrawn for at least about 24 weeks.
57. The method of any of the preceding paragraphs, wherein there is no substantial increase in serum level of liver alanine aminotransferase (ALT) enzyme following the AAV vector administration.
58. A method of treating Pompe disease in a subject, comprising administering to a subject who is being treated for Pompe disease with long-term GAA enzyme replacement therapy (ERT), a pharmaceutical composition comprising a recombinant adeno-associated virus (AAV) vector comprising in its genome, a heterologous nucleic acid sequence encoding a polypeptide comprising an alpha-glucosidase (GAA) polypeptide in expressible form at a dosage of between about 1.6 e¹² vg/kg to about 1.6e¹³ vg/kg, wherein the heterologous nucleic acid is operatively linked to a liver-specific promoter, and wherein the administration of long-term GAA enzyme replacement therapy (ERT) is withdrawn on the same day (d1), the day after, at least the day before, or anytime between a day and about 26 weeks, or anytime between a day and about 26 weeks after the administration of the recombinant AAV, and wherein the subject obtains a blood serum level of GAA expressed by the AAV at a pharmacological activity is at least 165 nmol/ml/hr of at least within two weeks of administration.
59. The method of paragraph 58, wherein the subject is administered methotrexate and prednisone for immune modulation.
60. the method of paragraph 59, where methotrexate is administered at an initial dose of 30 mg or less/week.
61. The method of paragraph 60, wherein methotrexate is administered at an initial dose of between about 5 and 30 mg/week.
62. The method of paragraph 61, wherein the methotrexate is administered at an initial dose of between 5 and 7.5 mg/week.
63. The method of any one of paragraphs 59-62, wherein the initial dose is tapered until the dose reaches 0.
64. The method of paragraph 63, wherein tapering is by a decrease of 2.5 mg/week to begin after 12 weeks.
65. The method of any one of paragraphs 59-64, wherein the methotrexate is administered once/week with 1 mg/day folic acid being administrated on non-methotrexate dosing days.
66. The method of any one of paragraphs 59-61, wherein the prednisone is administered at an initial dose of 60 mg or less.
67. The method of paragraph 66, wherein the prednisone administration is begun 24 hours prior to the AAV administration.
68. The method of any one of paragraphs 59-67, wherein the methotrexate administration is begun 1 hour prior to AAV administration.
69. The method of any one of paragraphs 59-68, wherein T cell reactivity to vector capsid is substantially low.
70. The method of paragraph 69, wherein T cell reactivity is below a threshold range of >40 to <129/sfu/million.
71. The method of any one of paragraphs 59-68, wherein there is no substantial increase in serum level of liver alanine aminotransferase (ALT) enzyme following the AAV vector administration.
72. The method of any one of paragraphs 59-71, wherein the pharmaceutical composition comprises a dose dosage of rAAV of 1.6 e¹² vg/kg, 5e¹² vg/kg, or 1.6e¹³ vg/kg.
73. The method of any one of the above paragraphs, wherein the pharmaceutical composition comprises a dose of rAAV sufficient to achieve serum level of hGAA expressed by the rAAV at a pharmacological activity range from at least about 165 to ≤2,260 nmol/ml/hr.
74. The method of any one of paragraphs 59-73, wherein the pharmaceutical composition comprises a dose of rAAV sufficient to achieve serum level of hGAA expressed by the rAAV at a pharmacological activity range from at least about 410 to 1386 nmol/ml/hr.
75. The method of any one of paragraphs 59-74, wherein the pharmaceutical composition comprises a dose of rAAV that is sufficient to express GAA to achieve clinical stability in the subject in one or more symptoms of Pompe disease, wherein clinical stability is selected from the group consisting of a) not exceeding a 15% decrease in Forced Vital Capacity (FVC) over two consecutive assessments, measured no less than 3-months apart, b) not exceeding a 12% decrease in the 6MWT over two consecutive assessment, measured no less than 3-months apart, c) not exceeding a 43-meter decrease in the 6MWT over two consecutive assessments, measured no less than 3-months apart; d) tissue GAA content at or above baseline by 24 weeks after administration; e) tissue glycogen content at or below baseline by 24 weeks post administration.
76. The method of any one of paragraphs 59-75, wherein the dose of rAAV is sufficient to improve clinical outcome in one or more symptoms of Pompe disease, where the clinical outcome is selected from the group consisting of a) an improved Forced Vital Capacity (FVC) over two consecutive assessments, measured no less than 3-months apart, and b) improved 6MWT over two consecutive assessment, measured no less than 3-months apart, and c) improved decrease in the 6MWT over two consecutive assessments, measured no less than 3-months apart.
77. The method of paragraph 76, wherein the dose of rAAV is between 5E12 vg/kg and 1.6E13 vg/kg, and the clinical outcome of improved 6MWT is an increase from baseline of between 3m and 86 m.
78. The method of paragraph 77, wherein the increase from baseline is observed by 8 weeks post administration
79. The method of any one of paragraphs 59-78, wherein GAA activity in muscle is increased by at least 2 fold than the level before the AAV vector was administered.
80. The method of any one of paragraphs 59-79, wherein GAA activity in muscle is increased by at least 2 fold than the level after the long term ERT was withdrawn for at least about 24 weeks.
81. The method of any one of paragraphs 59-80 wherein ERT is permanently ended.
82. The method of any one of paragraphs 59-81, further comprising administering complementary ERT after an extended period of time following the administration of the AAV vector.
83. The method of paragraph 82, wherein the extended period of time is about 25 weeks, about 97 weeks, about 107 weeks after the administration of the AAV vector.
84. The method of paragraph 82, wherein the extended period of time is at least 1 year following the administration of the AAV vector.
85. The method of paragraph 82, wherein the extended period of time is between 12-18 months after the administration of the AAV vector.
86. The method of paragraph 82, wherein the extended period of time is at least 6-months after the administration of the AAV vector.
87. The method of any one of paragraphs 82-86, wherein the complementary ERT is administered at a lower frequency and/or dosage than the administration of the long-term ERT.
88. The method of paragraph 82, wherein the complementary ERT is administered at regular intervals.
89. The method of paragraph 88, wherein the complementary ERT is suspended for an extended period of time of at least 10 weeks.
90. The method of paragraph 82, wherein the complementary ERT administration is sporadic.

### EXAMPLES

### Phase 1 Study of Gene Therapy in Late-Onset Pompe Disease: Analyses of Safety, Bioactivity, and Secondary Endpoints

### Example 1: Cohort 1

### Introduction

Presented herein are results of human clinical trial data designed to assess expression and therapeutic efficacy of AAV-mediated gene transfer to express GAA after systemic injection of AAV8 encoding GAA protein to treat Pompe disease in human subjects. The program was established to assess the safety and efficacy of treatment of Pompe disease with ACTUS-101. The results disclosed herein demonstrate that a single infusion of ACTUS-101 results in expression of GAA, which effectively removes glycogen and eliminates or reduces the need for costly and frequent ERT infusions by creating a stable liver depot for GAA production. These results provide significant advancements in the treatment of Pompe disease.

### Background

Long-term sustained benefit from a single injection of ACTUS-101 is strongly supported by data in GAA knockout (GAA KO) mice. ACTUS-101 injected by tail vein resulted in stable GAA secretion from the liver into the bloodstream for many months (Franco et al. 2005; Sun et al. 2006). Moreover, an analogous approach to gene transfer resulted in successful treatment of dogs with hemophilia B for up to 8 years (Niemeyer et al. 2009). If similarly effective in patients with Pompe disease, gene transfer will address the inherent limitations of ERT including the need for frequent infusions and the high cost of ERT (Desnick 2004). Lower doses of ACTUS-101 (2E10 vector genomes (vg), equivalent to 8E11 vg/kg bodyweight), induced immune tolerance to rhGAA and enhanced the efficacy of ERT in GAA KO mice (Sun et al. 2007; Sun et al. 2010).

From a clinical perspective, long-term treatment of Pompe patients with ERT has had limited success. The process or ERT is costly, unpleasant, the administration process carries risks and is time consuming for the patient. The potential for having or developing antibodies to GAA which ultimately effect the efficacy of the therapy also exists. In the IOPD subset of patients, many experience high, sustained anti-rhGAA antibody titers (HSAT) believed to reduce the effectiveness of enzyme delivery by receptor uptake. CRIM negative patients (patients who lack any residual GAA protein) develop HSAT, and a subset of CRIM positive patients who also develop high or sustained intermediate titers suffer greatly increased mortality (Banugaria et al. 2011). Furthermore, the use of immunosuppression to prevent antibody formation in patients at risk for HSAT significantly prolonged survival and confirmed the clinical relevance of HSAT (Mendelsohn et al. 2009; Banugaria et al. 2011).

Gene therapy with an adeno-associated virus serotype 8 (AAV8) vector (AAV8-LSPhGAA) could eliminate the need for enzyme replacement therapy (ERT) by creating a liver depot for GAA production.

### Objectives

The primary study objective was to evaluate the safety of AAV8-LSPhGAA (ACTUS 101) in adult subjects as assessed by the incidence of adverse events (AEs), serious AEs (SAEs), and clinical laboratory abnormalities. Secondary objectives included six-minute walk test distance (6MWT), forced vital capacity (FVC), serum GAA activity, and muscle GAA activity and glycogen content.

### Results

The study was a single site, prospective, non-randomized, open-label 52-week trial with the objectives of assessing safety, bioactivity, and immune responses following treatment with ACTUS-101 in adult subjects with LOPD (NCT03533673). Subjects who were eligible to participate included males and females who were at least 18 years of age or older, had received ERT for the 104-week period immediately preceding study day 1, and had received a stable dose of ERT for the 52-week period immediately preceding dosing on study day 1. Eligible subjects also had ability to walk at least 100 meters on the 6 minute walk test (6MWT). A total of 3 subjects were sequentially enrolled. The initial demographics and baseline characteristics of the cohort are shown in FIG. 2.

To minimize the potential for adverse immunological reactions to the investigational agent, all subjects were tested for anti-AAV neutralizing antibody activity and received immunosuppression (steroid prophylaxis with oral prednisone) 24 hours prior to receiving ACTUS-101 on Study Day 1, which was continued for 15 weeks of the study with a tapering dose of 5 mg/day beginning week 5 following the administration.

Subjects who met all entry criteria reported to the Electromyography Laboratory (EMG) within 45 days prior to study day 1 for a muscle biopsy which was obtained 14 (±5) days after their last ERT treatment. This biopsy was obtained at a time corresponding to their trough concentration of their previous dose of ERT, and prior to resumption of their regularly scheduled dose of ERT.

On Study Day 1, all subjects received a single dose 1.6E12 vg/kg of ACTUS-101 by controlled intravenous infusion through an indwelling catheter placed into a peripheral vein. Following dosing, subjects were closely monitored overnight. Blood was drawn for testing prior to being discharged and throughout the course of the study (once weekly from Weeks 4 through 16). Subjects were contacted regularly to query for adverse events (Summary shown in FIG. 3). Safety evaluations and assessments of bioactivity were performed at weeks 24, 28, 30, 38 and 52 (more if clinically indicated).

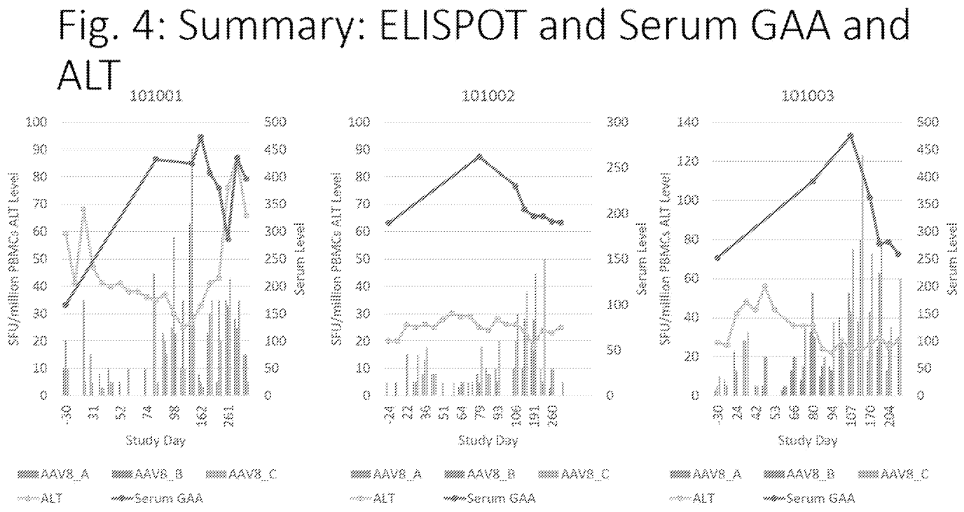

All subjects continued with their usual ERT for the initial 24 weeks of the study. Serum GAA was monitored at weekly intervals from Week 4 through Week 16 at the trough of ERT. ACTUS-101 activity was verified by detection of measurable levels of serum GAA at the ERT trough points (ie, weeks 4, 6, 8, 10, 12, 14 and 16). All subjects demonstrated sustained serum GAA levels from 101% to 235% above baseline through activity two weeks following the preceding ERT dose which confirmed bioactivity as GAA is not normally secreted into the blood. There were no treatment-related SAEs and laboratory assessments supported the safety of ACTUS-101 (AAV8-LSPhGAA) (FIG. 4 shows a summary of ELISPOT measuring reactivity to 3 polypeptides representing the AAV8 capsid protein, levels of serum GAA and alanine aminotransferase (ALT);

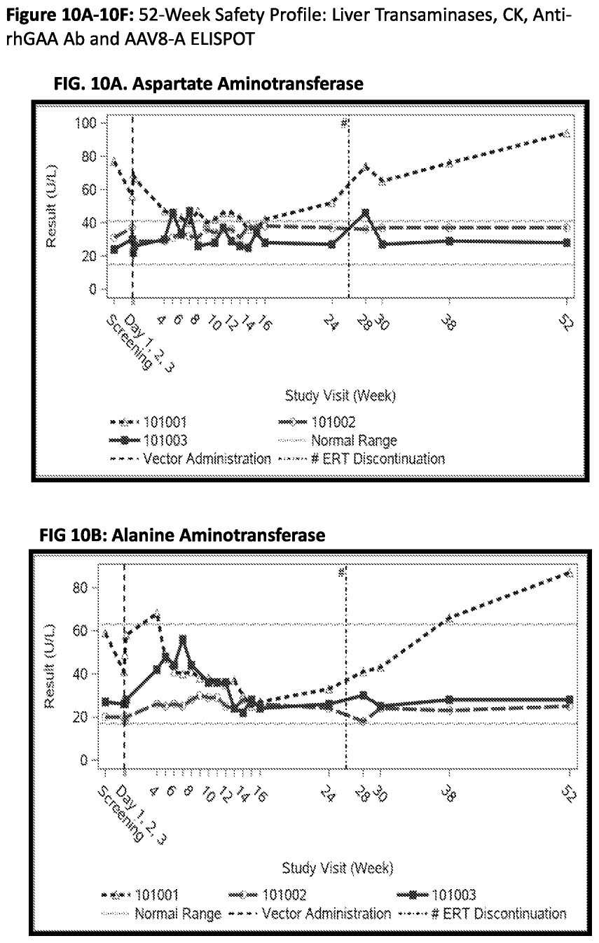

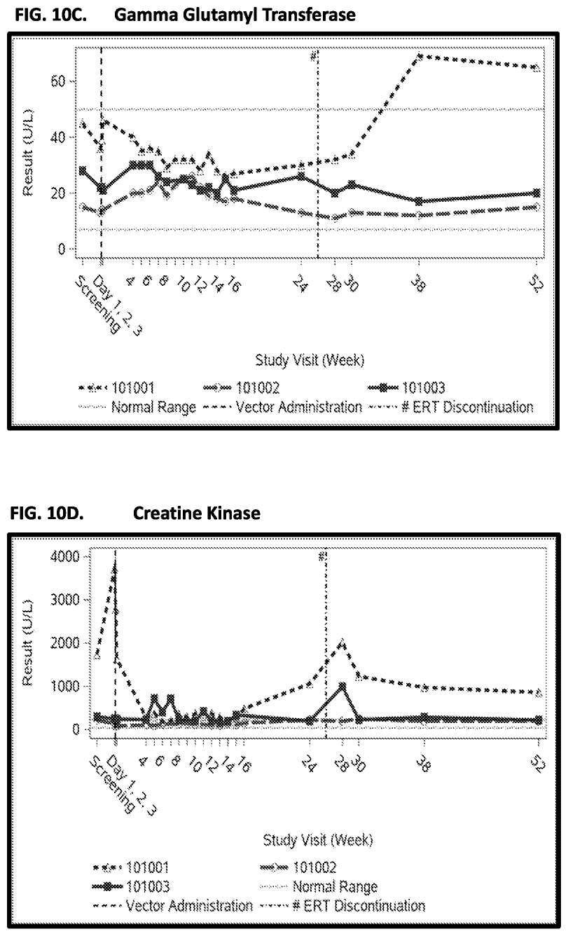

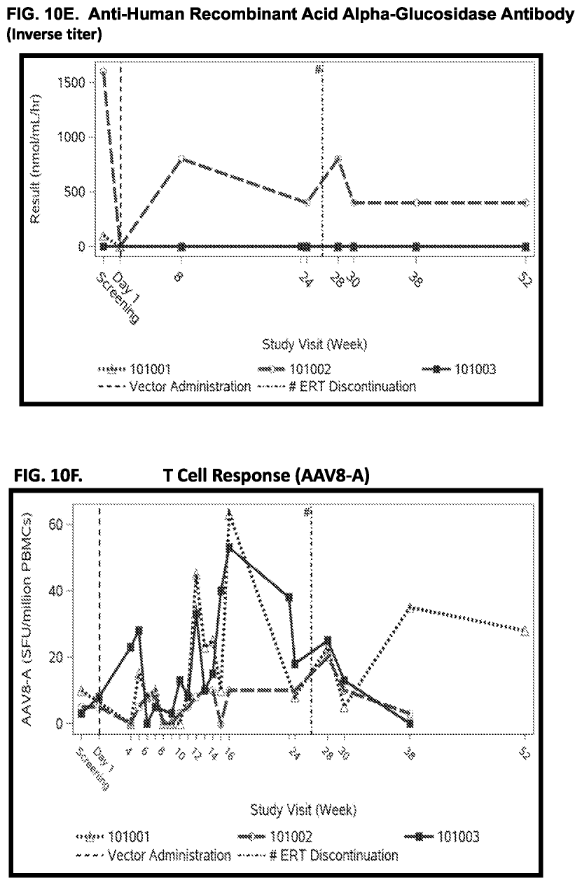

FIG. 10A-10F shows 52 week safety profile measuring liver transaminases, CK, anti-rhGAA antibody, and AAV8 A ELISPOT). No subject had elevated serum alanine aminotransferase, reflecting an absence of anti-AAV8 T-cell responses. Although there were no treatment-related SAEs, one subject experienced two moderate severity treatment-related AEs (headache).

The Week 24 visit occurred on the day prior to the subject's next scheduled dose of ERT (i.e., the day corresponding to the trough concentration of exogenous GAA activity supplied by ERT). At the Week 24 visit all subjects underwent a muscle biopsy for analysis of tissue GAA catalytic activity, GAA content and glycogen content. Blood samples were also taken and used to determine serum GAA catalytic activity. Subjects also underwent Pulmonary Function Testing, Muscle Status Testing, and Quality of Life Assessments. The results of a combination of tests (6MWT, GSGC, QMFT and FVC) indicated that all patients were eligible for withdrawal of ERT. The subjects then underwent their last usual ERT at week 24.

### Withdrawal of ERT

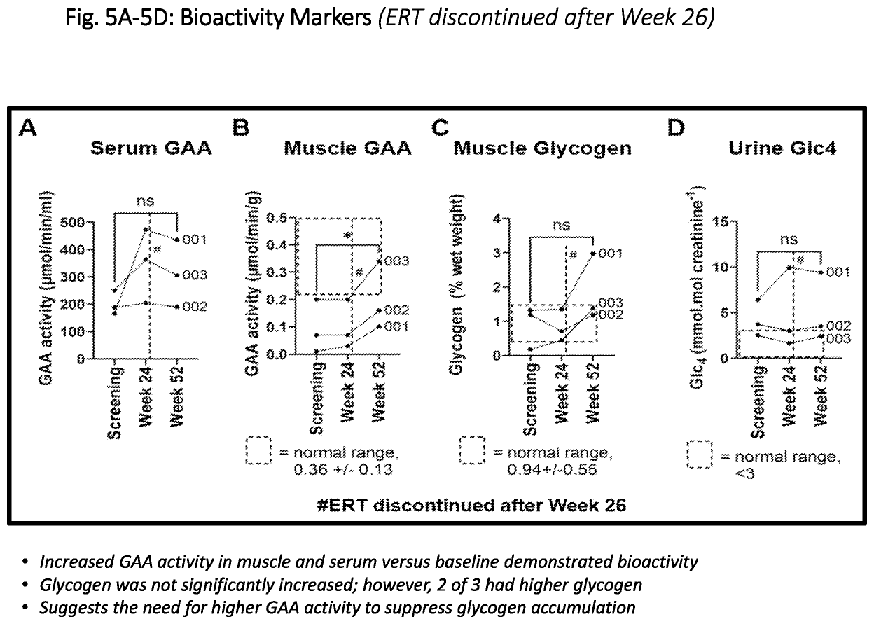

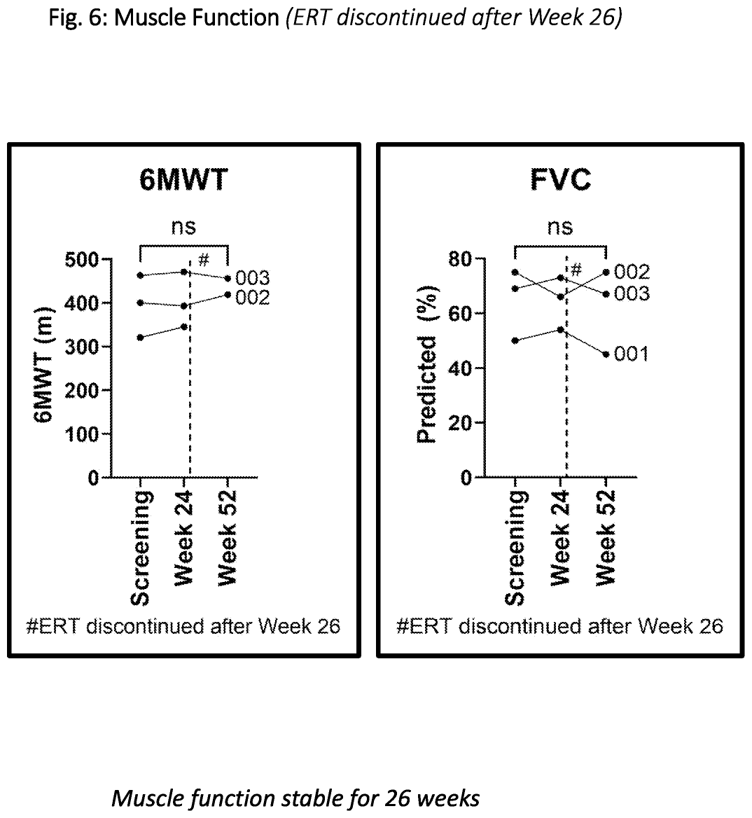

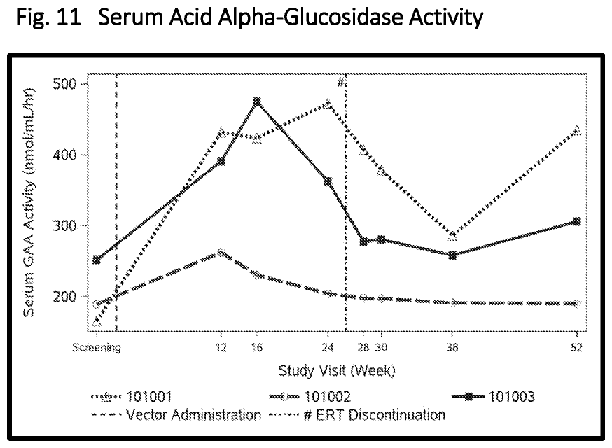

The data collected at Week 24 indicated all subjects met criteria for ERT withdrawal based on that data relative to their screening values (Table 2): (1) quantifiable GAA catalytic activity (i.e., endogenously expressed GAA activity as a result of dosing ACTUS-101) in their serum at Weeks 12, 16, and 24, and (2) no clinically significant declines in two or more parameters on two consecutive occasions (i.e., Week 8 and Week 24) in 6MWT and FVC from pulmonary function testing performed in the upright position. In light of this, all subjects had their ERT suspended at week 26. Increased GAA activity in muscle and serum versus baseline demonstrated continued bioactivity (FIGS. 5 and 11). Continued monitoring of the subjects indicated all subjects met the established criteria to remain off ERT (Table 2).

TABLE 2
Criteria for Withdrawal and Re-initiation of ERT in Cohort 1

| | Acceptable score | |
|---|---|---|
| | to withdraw | Score to |
| Test | ERT (range) | Re-initiate ERT |
| GAA Catalytic | Above | N/A |
| Activity in serum | screening/baseline | |
| | AND ONE OF | |
| 6MWT (Lachmann and | ≤30 m decline from | >43 m decrease |
| Schoser 2013; van der | baseline | from baseline in 2 |
| Ploeg et al. 2010) | | consecutive assessments |
| | | no less than 3 mo apart |
| PFT (FVC) (Lachmann | ≤3% decline from | >15% decrease |
| and Schoser 2013) | baseline | from baseline in 2 |
| | | consecutive assessments |
| | | no less than 3 mo apart |

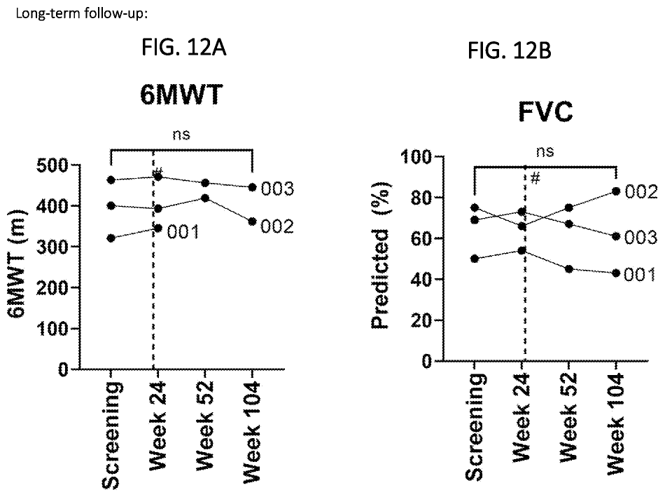

The eligibility to remain off ERT continued and was verified for all subjects at Weeks 52 and 104. One subject (subject 003) however opted to resume ERT at Week 97 (there was no measurable gain of function after ERT resumed for this subject, data not shown). Muscle biopsy at Week 24 revealed a large decrease (−40%) in muscle glycogen content for one subject (FIG. 5). At Week 52 serum GAA and muscle GAA activity for the entire cohort was significantly increased in comparison with baseline (p<0.05) (FIG. 5). One subject had a 10-fold increase in muscle GAA activity from 2.8% to 28% of normal, the second increased from 19% to 44% of normal, and the third increased from 56% to 94% of normal. Muscle glycogen was not significantly increased, although 2 of 3 had higher glycogen levels compared to initial screening levels (FIG. 5), suggesting a need for higher GAA activity to suppress glycogen accumulation. Measurements of possible detrimental effects from the vector were encouraging. T cell reactivity to the AAV capsid remained substantially low. Liver transaminase Alt maintained at low levels across the study period for two of the subjects. Taken together, these results demonstrate that the single dose of ACTUS-101 was safe and sufficient to allow a subject to eliminate long term ERT indefinitely based on the indicated criteria. Although a subject may elect to resume ERT for various reasons, these results demonstrate that even such subjects can have reduced frequency and/or reduced amounts of ERT administration, contributing significantly to quality of life. In particular, a long term follow-up of subjects that went without ERT (i.e. ERT withdrawal) for the period of 24 to 104 weeks had a stable 6MWT for up to 104 weeks (FIG. 12A), and stable FVC (forced vital capacity), or decrease in FVC at 104 weeks post administration, demonstrating that long-term ERT withdrawal is feasible in subjects who receive a single dose 1.6E12 vg/kg of ACTUS-101 (AAV8-LSPhGAA) (FIG. 12B).

Patient 101003 opted to resume ERT at 97 weeks, Patient 101001 opted to resume ERT at 110 weeks. Patient 101002 has elected to not receive any additional ERT to date (ERT withdrawal was Jul. 25, 2019), further indicating that complete ERT withdrawal is feasible in some subjects who receive a single dose 1.6E12 vg/kg of ACTUS-101 (AAV8-LSPhGAA).

### Discussion

Based on results obtained in nonclinical models, the 1.6E12 vg/kg dose used in this trial was not expected to support the permanent withdrawal of ERT. Information gained from humanized mice (Vercauteren et.al. 2016) indicated that there is a 20-fold differential in transduction efficiency with AAV8 between mouse and human hepatocytes. This difference was observed in multiple preclinical studies and in clinical trials using AAV8-based vectors for the treatment of hemophilia A and B (Nathwani et.al. 2014, 2011). Using a 20-fold differential, it was thought that the dose needed for ACTUS-101 to exert biochemical correction in humans and to allow the permanent withdrawal of ERT would be as high as 1.6E13 vg/kg (i.e., 20-fold above the 8E11 vg/kg dose that led to biochemical correction in the GAA KO mouse). In light of these expectations, the ability of the 1.6E12 vg/kg dose to allow suspension of the usual ERT (long term ERT) for an extended period of time, or even indefinitely were somewhat surprising. These results indicate that subjects who eventually resume ERT (e.g., out of personal choice or due to a future clinical indicator) will have the ability to significantly reduce the dosage and/or frequency of that ERT. Furthermore, the results indicate that the subjects who may resume ERT will not suffer any serious consequences, short term or long term, from an extended period of suspension of ERT (e.g., several weeks to months), allowing for an ERT holiday. The results of this study further support the cessation of ERT at the time of vector administration rather than continuing ERT for several weeks.

The dosing frequency of ERT carries a high treatment burden for the patient and caregiver, with both missing school and/or work days. Each infusion takes up to 8 h weekly or every other week and must be performed by a qualified practitioner typically at a medical treatment facility. A one-time treatment of ACTUS-101 can significantly reduce the need for infusions via central or peripheral catheters. Furthermore, the consequences of missing a scheduled ERT dose would be significantly mitigated. In addition, the available option of a holiday from the ERT provides a subject considerable flexibility in patient's lifestyle, and as such provide a substantial increase in their quality of life.

### Materials and Methods

Subject Selection based on Antibody Screening. Subjects were pre-screened for any antibodies against AAV8 as the presence of such antibodies would prevent ACTUS-101 vector from being effective. Preliminary data from screening protocol NCT03285126 indicated that the prevalence of pre-existing immunity against AAV8 among subjects followed at the Duke Metabolic Clinic is 68.8% (11/16). Therefore, potential subjects were screened to identify subjects with no antibodies against AAV8.

ACTUS-101 (Vector) Administration. There was a stagger of 4 weeks between each subject. Participants were admitted overnight to the DEPRU for vector administration. All subjects received a single dose 1.6E12 vg/kg of ACTUS-101 by controlled intravenous infusion through an indwelling catheter placed into a peripheral vein. Following administration of ACTUS-101 the subjects were closely monitored and remained overnight in the DEPRU and had blood drawn for safety testing prior to being discharged the following day (Day 2). All subjects returned to the outpatient clinic for safety testing on Day 3. They received a phone call at 5 days and two weeks later to query for adverse events. Adverse events and blood samples were collected by home health care nurse during weekly home visits at the start of Weeks 4 to week 16 to monitor for potential elevations in liver function tests. Home visits occurred any day (Monday-Thursday) during the specified week. All subjects returned to the outpatient clinic for safety evaluations and assessments of bioactivity at weeks 24, 28, 30, 38 and 52 and more if clinically indicated. The Week 24 visit occurred on the day prior to the subject's next scheduled dose of ERT (i.e., the day corresponding to the trough concentration of exogenous GAA activity supplied by ERT). At the Week 24 visit all subjects underwent a muscle biopsy (taken from the vastus lateralis) for the analysis of tissue GAA catalytic activity and GAA content (determined by western blot) and glycogen content. Subjects also donated a blood sample for the determination of serum GAA catalytic activity and underwent Pulmonary Function Testing, Muscle Status Testing, and Quality of Life Assessments; the 6MWT, GSGC, QMFT and FVC informed the decision to whether or not withdraw ERT as outlined in Table 2. All assessments were done prior to obtaining the muscle biopsy. Follow-up visits to assess long-term safety of gene transfer will occur for a total of 5 years. A detailed schedule of study visits, tests, and procedures is outlined in Appendix 1. The follow-up schedule post Week 52 is outlined in Appendix 2.

Doses of ACTUS-101 (Vector). Dosages of vector was 1.6E12 vg/kg with an N of 3.

ACTUS-101. ACTUS-101, also referred to as AAV8-LSPhGAA, is a gene therapy product with an AAV8 capsid, a liver specific promoter (LSP) and the transgene for hGAA. It was administered as a one-time single IV administration. Upon administration, the AAV vector selectively expressed and secreted GAA from transduced hepatocytes. The primary mechanism of action was to secrete continuous low levels of endogenous GAA from the liver into the systemic circulation in order to provide therapeutic exposure levels of GAA to tissue (predominantly muscle), resulting in glycogen removal and restoration of cellular architecture and function. A secondary mechanism of action of ACTUS-101 was to mediate a regulatory T cell response in the host resulting in immunotolerance to the secreted GAA which itself is a potent immunogen. Inducing immune tolerance combats the potentially negative effects of ERT such as inhibition of receptor-mediated uptake of GAA by neutralizing antibody.

Study Criteria. The criteria for withdrawal required that an initial decline in either motor or pulmonary function was confirmed by a subsequent assessment performed no less than 3 months later. The three month interval between assessments was used to minimize the potential to make an erroneous decision to reinitiate ERT due to the inherent variability of motor and pulmonary function assessments while ensuring subject safety and minimizing Type I and Type II errors.

Bioactivity. Bioactivity was evaluated by the analysis of GAA catalytic activity in serum and muscle and glycogen and GAA content in muscle. Blood (for the determination of GAA catalytic activity in serum) was taken as outlined in Appendix 1, and will be taken in future as outlined in Appendix 2 for the 2 to 5 year follow-up. Muscle biopsies (taken from the vastus lateralis) were performed at Screening, Week 24, and Week 52 (i.e., the day before next scheduled ERT, visit minus 1 day if ERT was still being received).

Antibody Formation. The effect of gene transfer on antibody production was assessed. The presence of anti-rhGAA IgG antibody titer was monitored by Enzyme Linked Immuno-Sorbant Assay (ELISA) as outlined in Appendix 1 (FIG. 8) and Appendix 2 (FIG. 9).

Immunosuppression. Recent data indicate a risk for anti-AAV immune responses in the liver accompanied by transaminase elevations that might be prevented by initial immunosuppression (Mendell 2016). Prophylactic immunosuppression was begun approximately 24 hours prior to receiving ACTUS-101 and consisted of oral prednisone at 60 mg daily for four weeks followed by a 5 milligram taper each week for 11 weeks based upon the published methods of immunosuppression (Nathwani, Rosales, et al. 2011; Mendell et al. 2017). All subjects received the immunosuppression.

Safety. The potential adverse impact of gene transfer on the signs and symptoms of disease were assessed by Pulmonary Function Testing, Muscle Status Testing (Gross Motor Function Measure GMFM88, Gait Stairs Gower Chair GSGC with Timed Functional Tests TFTs, Grading of Functional Tests, Quick Motor Function Test QMFT), and the 6-Minute Walk Test. All assessments were performed as outlined in Appendix 1, or will be performed as outlined in Appendix 2.

Adverse Event Reporting. AEs were reported as specified in M-I-C-4-a of the NIH Guidelines for Research Involving Recombinant DNA Molecules (https://osp.od.nih.gov/wp-content/uploads/NIH_Guidelines.html) to the NIH Office of Biotechnology Activities. All AEs and SAEs were attributed to either ACTUS-101, concomitant medications (e.g., ERT, oral prednisone) or procedures related to the administration of ACTUS-101 or concomitant medications or specific testing.

Quality of Life (QoL). Assessments included the Fatigue Severity Scale, Rasch Pompe Specific Activity Scale, and the National Institutes of Health Patient Reported Outcomes Measurement Information Systems (NIH PROMIS) short forms for fatigue, physical function, and pain interference that are relevant to the LOPD population. In addition, the Hemophilia Caregiver Impact (HCI) measure was included to understand patient treatment burden for ERT. Subjects completed or will complete these instruments as outlined in Appendix 1 and Appendix 2, respectively.

Blood Samples. Blood samples were obtained at all study visits for the following analyses: a) Serum GAA catalytic activity. b) Safety Labs: Hematology and Chemistry to consist of: Complete Blood Count (CBC), Chemistry panel (including electrolytes, BUN, creatinine, glucose and HbAlc), Liver function tests (including albumin, aspartate aminotransferase (AST), alanine aminotransferase (ALT), gamma-glutamyltransferase (GGT), and total bilirubin), Creatine kinase (CK) c) Anti-rhGAA Antibody (IgG) Titers/Inhibitory Antibody Monitoring. d) ELISA for antibodies to AAV8 capsid proteins e) T Cell Responses, performed by ELISpot assays using peptides (11 to 15 amino acids in length) that represent the length of the AAV8 capsid protein. (See Appendix 1 and 2 for timing). f) Serum Pregnancy test (females of childbearing potential).

Urine Samples. Urine was collected for Glc4, AAV8 viral capsid and for pregnancy testing (for females of childbearing potential).

Tissue Samples. Muscle biopsy for tissue morphology (histology), quantification of glycogen content, GAA catalytic activity and analysis of GAA precursors and active isoforms. Biopsies were taken from the thigh (vastus lateralis). The needle muscle biopsy was performed under local anesthesia. Risks associated with the procedure include hematoma, infection and scar formation.

Muscle strength and function, and pulmonary function testing in LOPD. Muscle strength and muscle function were evaluated with measures of impairment, function, participation, and quality of life. Functional strength measures included the GMFM88 (Russell et al. 1989), classic upper and lower extremity functional grades, (Brooke et al. 1983; Brooke et al. 1987), as well as timed (Moxley 1990), and graded functional tests which were included to allow scoring with the GSGC (Angelini et al. 2009) and the Quick Motor Function Test (van Capelle et al. 2012) all of which have been used with LOPD, and which offer quantitative assessment of activities of elevation against gravity and gait. Endurance was assessed with the 6MWT (ATS statement: guidelines for the six-minute walk test’ 2002). Pulmonary function was assessed in both the supine and upright positions to increase sensitivity for abnormalities detected in Pompe disease (van Capelle et al. 2010). Pulmonary function testing included measuring forced vital capacity (FVC), forced expiratory volume in the first second (FEV1), maximum expiratory pressure (MEP), and maximum inspiratory pressure (MIP), and was measured by electronic spirometer as previously described (Brooke et al. 1983). FVC and FEV1 were assessed in both the supine and upright positions to increase sensitivity for abnormalities detected in Pompe disease; only upright values were used for assessment of safety (van der Beek et al. 2011).

Data Collection for Long Term Follow-up Subjects. Subjects in this study of gene transfer for Pompe disease will be followed for 5 years. All subjects will be seen every 4 months for Years 2 through 5. These visits will include those outlined in the Schedule for Research Testing, Appendix 2.

Management of Presumed Vector-Related Hepatitis. Subclinical transient hepatitis (liver transaminase elevations up to 5×ULN) has been observed in subjects of liver-directed AAV trials for hemophilia B (2E12 vg/kg) (Manno et al. 2006; Nathwani, Tuddenham, et al. 2011). An apparent cytotoxic T lymphocyte (CTL) response directed against vector-transduced hepatocytes was dependent upon the dose of inciting antigen (the AAV capsid) and has not been seen at the low doses in either trial. Based upon the approach of Nathwani and colleagues, who administered an AAV8 vector to subjects with hemophilia (Nathwani, Tuddenham, et al. 2011), the following was planned: Although weekly measurements of ALT and AST will be monitored, patients with Pompe disease have chronic elevations of these enzymes due to muscle pathology. Thus, any subject who develops a GGT level>1.5×ULN during the study must have the test repeated, and a diagnostic workup of potential infectious and toxic/metabolic causes of hepatitis will be pursued as soon as possible, including consultation with a hepatologist. Safety laboratories including GGT, ALT, and AST were monitored weekly beginning at Week 4 and ending at Week 16. If no other cause for hepatitis is found, the presumed cause is CTL against the AAV8 capsid proteins. Treatment with oral prednisone will be initiated according to the published methods of immunosuppression (Nathwani, Rosales, et al. 2011; Mendell et al. 2017). The initial dose will be oral prednisone, 60 mg p.o. daily for 4 weeks, followed by a 5 mg taper each week for 11 weeks. Continuation of oral prednisone beyond 15 weeks will be guided by ongoing medical judgement.

Other Assessments. (See Appendix 1 and 2 for timing). Other Research Activities performed were Medical History, Vital Signs, Physical Examination, AE Review, and Electrocardiogram (ECG).

Safety and Clinical Analysis. The number of AEs and SAEs was summarized overall, by severity. Laboratory data, such as hematology and serum chemistry data were tabulated by dosing group. Summary statistics for changes from baseline will be presented. Continuous laboratory measurements were described at each visit using univariable descriptive statistics (mean, median, etc.); observed values and changes from baseline were summarized. Shift analysis for critical laboratory data were performed. Lab tests reflective of liver toxicity (e.g., GGT) were further summarized in terms of the most extreme values and largest changes from baseline (in the appropriate direction) observed from start of study drug through the completion of the Week 52 visit. For the primary hypothesis, a dose was defined to exceed the maximally tolerated dose (MTD) if either of the following occurred: a) At least two of three subjects on that dose have DLT or b) If exactly one of the first three subjects has DLT, and DLT occurred in one of a possible two additional subjects accrued to this dose. The operational definition of DLT was a grade 3 AE that was deemed to be related to ACTUS-101 and occurred within 16 weeks of vector administration. The study was to be temporarily closed if the dose for a potential subject could not be defined, that is, when the third subject at a dose level has been accrued, but DLT (yes vs. no) had not been established for all of the subjects. If the first level exceeded the MTD, the investigators were to consider the possibility of studying one lower level in the same way. For the secondary hypothesis, GAA catalytic activity in the subjects' serum were analyzed before versus after by McNemar's test. Any changes in repeated serum GAA catalytic levels were analyzed by linear regression with dose level as a covariate.

Adverse Event. An adverse event (AE) was considered to be any symptom, sign, illness or experience that develops or worsens in severity during the course of the study whether or not considered related to investigational product. Intercurrent illnesses or injuries were regarded as adverse events. Abnormal results of diagnostic procedures were considered to be adverse events if the abnormality:
Results in study withdrawal
was associated with a serious adverse event
was associated with clinical signs or symptoms
led to additional treatment or to further diagnostic tests
was considered by the investigator to be of clinical significance

Serious Adverse Event. Adverse events were classified as serious or non-serious. A serious adverse event was considered any AE that was:
fatal
life-threatening
required or prolonged hospital stay
resulted in persistent or significant disability or incapacity
a congenital anomaly or birth defect
an important medical event
   All adverse events that did not meet any of the criteria for serious were regarded as non-serious adverse events.

Important Medical Event. Important medical events were considered those that may not have been immediately life threatening but were clearly of major clinical significance. They may have jeopardized the subject and may have required intervention to prevent one of the other serious outcomes noted above. For example, drug overdose or abuse, a seizure that did not result in in-patient hospitalization or intensive treatment of bronchospasm in an emergency department was typically considered serious.

Adverse Event Reporting Period. The study period during which adverse events must be reported is normally defined as the period from the initiation of any study procedures to the end of the study treatment follow-up. For this study, the study treatment follow-up was defined as 5 years following the administration of study treatment.

Preexisting Condition. A preexisting condition not related to Pompe disease was considered to be one that was present at the start of the study (i.e., was recorded during screening and discovered prior to administration of study drug). A preexisting condition was recorded as an adverse event if the frequency, intensity, or the character of the condition worsened during the study period.

General Physical Examination Findings. At screening, any clinically significant abnormality not related to Pompe disease was recorded as a preexisting condition. At the end of the study, any new clinically significant findings/abnormalities that met the definition of an adverse event were also recorded and documented as an adverse event.

Post-study Adverse Event. All unresolved adverse events were and will be followed by the investigator until the events were/are resolved, the subject is lost to follow-up, or the adverse event is otherwise explained. At the last scheduled visit, the investigator will instruct each subject to report any subsequent event(s) that the subject, or the subject's personal physician, believes might reasonably be related to participation in this study. After the 5-year follow-up, the investigator should notify the study sponsor of any death or adverse event occurring at any time after a subject has discontinued or terminated study participation that may reasonably be related to this study. The sponsor will also be notified if the investigator becomes aware of the development of cancer or of a congenital anomaly in a subsequently conceived offspring of a subject that has participated in this study.

Abnormal Laboratory Values. A clinical laboratory abnormality was documented as an adverse event if any one of the following conditions were met: a) The laboratory abnormality was not otherwise refuted by a repeat test to confirm the abnormality, worsened over baseline, was unrelated to the underlying disease process and was regarded as clinically significant by the investigator, b) The abnormality suggested a disease and/or organ toxicity c) The abnormality was of a degree that required active management; e.g. more frequent follow-up assessments, further diagnostic investigation, etc.

Hospitalization, Prolonged Hospitalization or Surgery. Any adverse event that resulted in hospitalization or prolonged hospitalization were documented and reported as a serious adverse event unless specifically instructed otherwise in this protocol. Any condition responsible for surgery was documented as an adverse event if the condition met the criteria for an adverse event. Neither the condition, hospitalization, prolonged hospitalization, nor surgery were reported as an adverse event in the following circumstances: a) Hospitalization or prolonged hospitalization for diagnostic or elective surgical procedures for a preexisting condition. Surgery was not reported as an outcome of an adverse event if the purpose of the surgery was elective or diagnostic and the subject did not experience unexpected, and serious events related to the surgery, b) Hospitalization or prolonged hospitalization for therapy of the target disease of the study, unless it was a worsening or increase in frequency of hospital admissions as judged by the clinical investigator.

Recording and Attribution of Adverse Events. At each contact with the subject, the investigator sought or will seek information on adverse events by specific questioning and, as appropriate, by examination. Information on all adverse events was or will be recorded immediately in the source document, and in the case report form (CRF). All clearly related signs, symptoms, and abnormal diagnostic procedure results were or will be recorded in the source document, and grouped under one diagnosis. All AEs and SAEs were or will be attributed to either ACTUS-101, concomitant medications (e.g, ERT, oral prednisone) or procedures related to the administration of ACTUS-101 or specific testing. All adverse events occurring during the study period were recorded. The clinical course of each event was followed until resolution, stabilization, or until it has been determined that the study treatment or participation is not the cause. Serious adverse events that are still ongoing at the end of the study period will be followed up to determine the final outcome. Any serious adverse event that occurs after the 5 years and is considered to be possibly related to the study treatment or study participation should be recorded and reported to the investigator.

Stopping Rules. Dose-limiting toxicity was defined as any adverse event of grade 3 or higher that is possibly, probably, or definitely related to ACTUS-101. There was at least 4 weeks between dosing of the subjects within Cohort 1. The allowance of at least two weeks between dosing of subjects provided time for review of the safety analysis of all the subjects within the cohort. The appropriate DSMB and FDA were conferred with on all grade 3 or higher adverse events that possibly, probably, or definitely related to the study agent.

Resumption of Enzyme Replacement Therapy. To be considered for resumption of ERT, other than as requested by the subject, a subject must have exhibited consistent clinically significant declines as defined herein on two consecutive occasions for at least two parameters including 6MWT, GSGC, QMFT, and Forced Vital Capacity performed in the upright position. Resumption of ERT post Week 52 will be considered at each 4 month visit (or any unscheduled visits) through years 2 to 5 based on the same criteria, that is, clinically significant declines on two consecutive occasions.

### Example 2: Cohort 2

### Introduction

This cohort was tested for safety and effectiveness of a high dose of the AAV-GAA.

### Results

There were three subjects in this cohort. The same procedures were used for this second cohort as in Example 1, with the exception of dosage of AAV-GAA, timing of ERT withdrawal, and the use of methyl prednisolone instead of prednisone. In addition, subject 006 did not receive any corticosteroid (e.g., methyl prednisolone) due to observance of lack of tolerance in subjects 004 and 005. This cohort used a dosage of 1.6E13 vg/kg. ERT was withdrawn at or before the same day as the AAV administration (Day 1). Transaminitis was observed in all subjects beginning shortly after week 8. Prior to the peak in the observed transaminitis, GAA serum concentrations ranged from 410 to 1,386 nmol/ml/hr. All subjects exhibited gains in motor function, with a change in baseline in the 6 minute walk test of 3 m, 20 m, and 86 m respectively, at the 8 week testing point (shown below in Table 8).

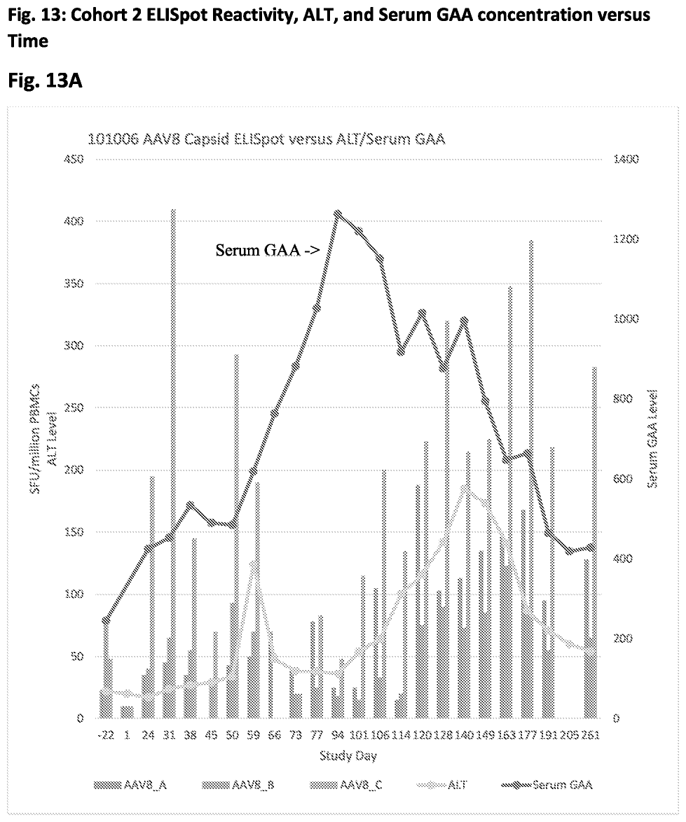

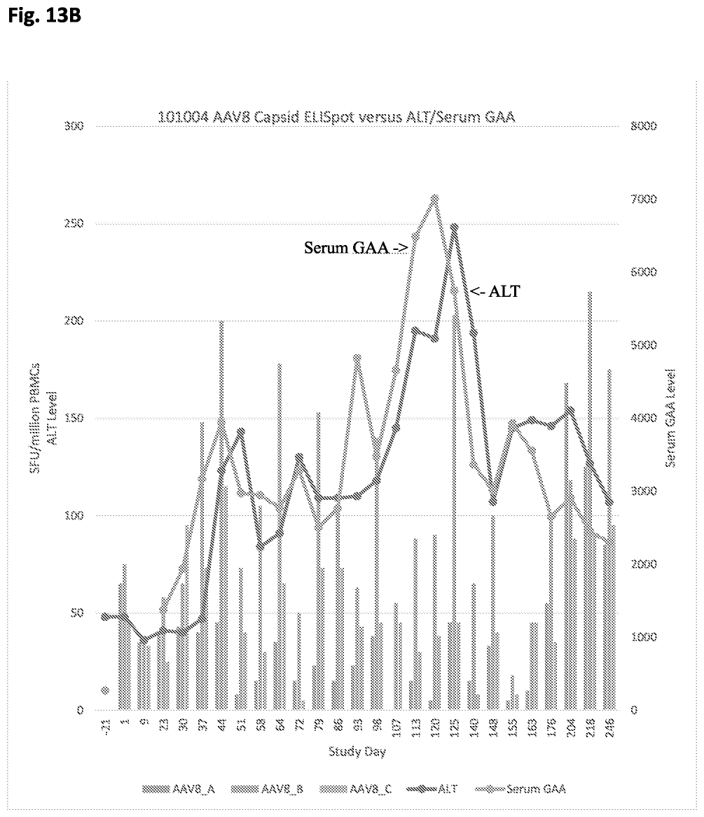

All subjects experienced transaminitis shortly after week 8 and began to lose transgene protein expression between weeks 13 and 16 (FIG. 13). Prior to the peak in the observed transaminitis, GAA serum concentrations ranged from 410 to 1,386 nmol/ml/hr. This loss in transgene protein expression led to declines in their 6 minute walk test performance, which was drastically reduced by the 16 week testing point, with one falling below baseline. However, two of the subject still were able to perform slightly above baseline at this point, performing at 20 and 18 meter over baseline, respectively. The reduction in the 6 minute walk test corresponded with the fall off of transgene expression brought about by the transaminitis.

TABLE 8

| Absolute Distance Walked (m) for | | | | | |
|---|---|---|---|---|---|
| Subjects in Cohort 2 (1.6E13 vg/kg) | | | | | |
| | | | 8-week | | 16-Week |
| | Baseline | 8-Week | Change v | 16-Week | Change v |
| Subject | Distance | Distance | Baseline | Distance | Baseline |
| Number | (m) | (m) | (m) | (m) | (m) |
| 004 | 501 | 521 | +20 | 481 | −20 |
| 005 | 221 | 307 | +86 | 241 | +20 |
| 006 | 107 | 110 | +3 | 125 | +18 |

All subjects eventually resumed ERT, with 004 resuming at 64 weeks, subject 005 resuming at 53 weeks and subject 6 resuming at 77 weeks.

### Discussion

All subjects gained in absolute distance walked from baseline at the week 8 analysis. All had been on ERT for 6 to 8 years. After this length of time on ERT no further gains in distance walked would be expected for heavily pre-treated adults with LOPD. This is based on the pharmaceutical literature and experience with alglucosidase alpha (lumizyme) in this population. To put things in perspective the 3 m gain by 006 is on pace with improvement seen in treatment naïve subjects who start ERT with lumizyme (the population giving the most robust response to ERT; clinical studies of 77 weeks in duration showed a gain of 31 m for the treated population during the primary observation period). The gains for 004 and especially 005 were completely unexpected and exceeded anything the physicians has seen to date. the next assessment was at week 16. Unfortunately, all experienced transaminitis shortly after week 8 and began to lose transgene protein expression which led to declines in walk test performance. However, even at week 16, with the transaminitis and fall off of transgene protein expression, subjects 005 and 006 still exhibited significant increase over baseline.

This higher dosage showed initially profound gains in motor function at the 8-week timepoint. This was prior to the development of transaminitis, which caused the serum GAA levels produced by the AAV-GAA to plummet, necessitating eventual resumption of ERT. The initially promising results indicate that the use of more effective methods of overcoming the subjects' immune response, e.g. with the use of different immune modulators such as methotrexate, will allow this treatment to provide patients with therapeutic effects that go well beyond ERT.

### Materials and Methods

Procedures were similar to those followed in Example 1 with the exception of the following.

Doses of ACTUS-101 (Vector). Dosages of vector was 1.6E13 vg/kg with an N of 3.

ERT Administration and Withdrawal. ERT was administered the same as in Example 1, except that it was ended just prior to the day of AAV administration (Day 1). ERT was eventually resumed at Day 53, 64 and 77.

Corticosteroid Administration. Methyl prednisolone was administered instead of prednisone. In addition, subject 006 did not receive any corticosteroid (e.g., methyl prednisolone) due to observance of lack of tolerance in subjects 004 and 005.

### Example 3: Cohort 3

### Introduction

A single subject was admitted into Cohort 3 for testing a mid-range dose of the AAV-GAA. The use of other immune modulators was also tested.

### Results

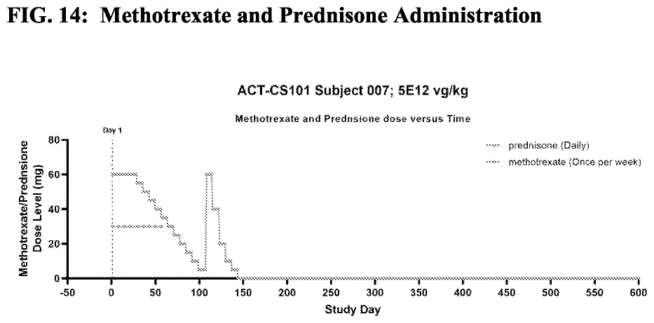

The same procedures were used as in Example 1, with the exception of cohort size, revised dosage, differing ERT withdrawal, and altered immunosuppressant. Cohort size was 1. Dosages of vector was 5E12 vg/kg. ERT was withdrawn the same day as the AAV administration (Day1). In addition, methotrexate was added to the prednisone as the immuno suppressant. The regimen of methotrexate and prednisone administration is represented in FIG. 14. Prednisone 60 mg/day was begun around 1 day prior to AAV-GAA administration, methotrexate 30 g/week was begun 1 hour prior to AAV-GAA administration. Folic acid (1 mg/day) was given orally on days that methotrexate was not administered to mitigate the methotrexate toxicity. Methotrexate (and folic acid) administration was continued for 8 weeks following the initial administration, at which time it was discontinued due to methotrexate associated pneumonitis, which resulted in the subject being hospitalized due to shortness of breath. Prednisone dose of 60 mg/day was continued until day 29, when a taper of 5 mg/week was begun, until day 106, where it reached 0. On day 110 the dose was increased from 0 back to 60 mg/day due to increased transaminitis. This was followed by a fairly rapid taper of 10 mg/week, until reaching 0 at day 145. The subject recovered completely from the methotrexate associated symptoms without further incident.

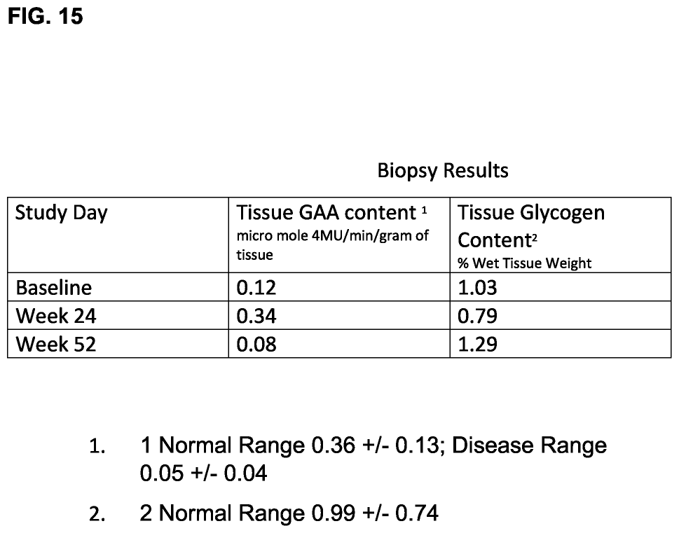

Biopsy was taken throughout the course of treatment to track tissue GAA content and tissue glycogen content. The data is presented in FIG. 15. Biopsy results at week 24 indicated tissue GAA content and tissue glycogen content improvement over ERT. Tissue GAA content (0.34 mol/min/gram tissue) was significantly above baseline (0.12 μmol/min/gram tissue) and tissue glycogen content (0.79% wet tissue weight) was below baseline (1.03% wet tissue weight). By week 52 the situation had reversed, with the tissue GAA content falling (0.08 μmol/min/gram tissue) below baseline, and the tissue glycogen content (1.29% wet tissue weight) rising above baseline, presumably due to the increased immune response to the AAV-GAA which reduced the serum GAA.

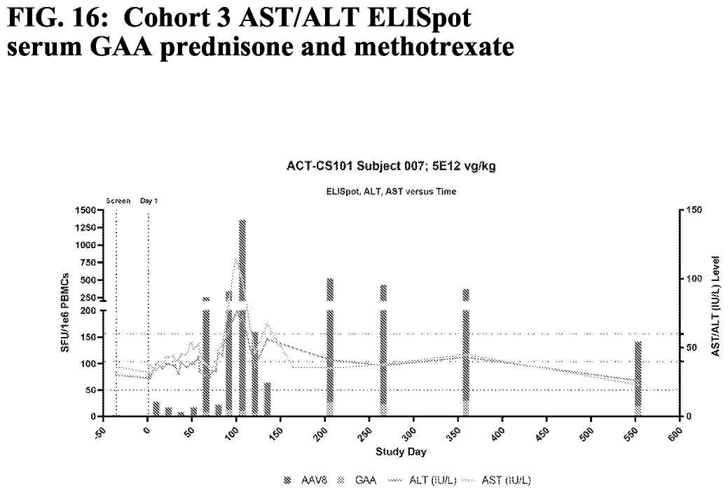

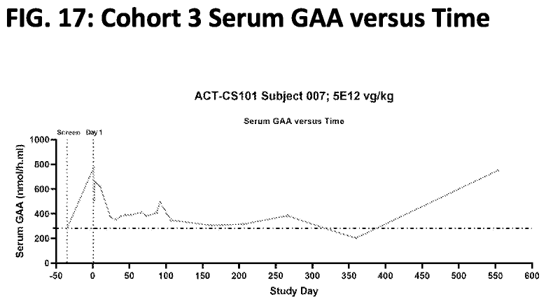

The T cell response to the vector and the transaminitis in the subject over the course of the study are shown in FIG. 16. T cell reactivity and transaminitis (AST/ALT increase) both begin to increase following the time the methotrexate administration was ended (around 56 weeks), and peak at the time of the final prednisone stepdown. They are reduced somewhat by the second round of prednisone, with transaminitis being brought to normal levels, but the T cell reactivity to the vector remaining high. The serum GAA of the subject over the course of the study is shown in FIG. 17. At Day 1, the serum GAA was high due the previous ERT. Serum GAA levels from the AAV-GAA initially increased and then fell, with the decrease corresponding to the time of increased T cell reactivity to the AAV-GAA. At around day 350, ERT was resumed, leading to another rise in the serum GAA levels.

### Discussion

The regimen was well tolerated until about study day 50 when the subject was hospitalized with shortness of breath due to methotrexate associated pneumonitis. At which time methotrexate was discontinued altogether but prednisone was maintained as described above. The subject recovered without further incident upon withdrawal of methotrexate.

Flow cytometric analysis and ELISpot analysis indicates that expansion of CD4 and CD8 positive T cells was completely suppressed while methotrexate was administered (FIG. 16 and data not shown). Transaminitis, resulting from capsid-directed T cell activity against transduced hepatocytes, was not observed through week 8 during methotrexate administration presumably due its suppressive effect on T cell replication. The absence of actively proliferating T cells was inferred by the absence of transaminitis and demonstrated by the absence of a double positive population of CD4 and CD8 T cells (cells negative for nuclear antigen ki67 and negative for cell membrane antigen CD71). Within two weeks of methotrexate withdrawal (week 10) CD4 and CD8 cell replication was apparent by flow cytometric analysis (by detection/appearance of the double positive population) and ELISpot analysis (FIG. 16). Transaminitis (clearly elevated AST and ALT) was observed at week 14 (FIG. 16) together with double positive T cells.

The data indicates that methotrexate is an effective non-steroidal alternative for either immune prophylaxis or for reactive treatment of transaminitis through its potent ability to suppress T cell replication. However, 30 mg/week was not tolerated. Methotrexate is commonly used in rheumatoid arthritis as an initial therapy. The 30 mg/week used in this study was at the higher end of the dose range used for rheumatoid arthritis. This dosage, although effective at first, was established here to be too aggressive. Still the positive results obtained early in the study suggest that a more moderate approach will have longer lasting effects. Lower dose ranges of methotrexate are viable for maintaining rheumatoid arthritis therapy, and should prove useful in mediating immune response in AAV-GAA therapy.

These studies suggest the use of a lower methotrexate dose, such as beginning with 5 to 7.5 mg/week, with 1 mg/folic acid on the remaining 6 days in each week. Tapering would begin after about 12 weeks, or as determined by patient tolerance, and follow a decrement of about 2.5 milligrams per week, with folic acid administration maintained throughout the taper. Tapering to 0 (for both methotrexate and folic acid) is the goal, however, maintaining low level methotrexate may be advantageous in some subjects.

These results indicate that the use of immunomodulators other than corticosteroids, such as methotrexate, will provide immunomodulation such that the AAV-GAA based therapy can produce therapeutic effects similar to and even superior to ERT.

### Materials and Methods

Procedures were similar to those followed in Example 1 with the exception of the following.

Doses of ACTUS-101 (Vector). Dosages of vector was 5E12 vg/kg with an N of 1.

ERT Administration and Withdrawal. ERT was administered the same as in Example 1, except that it was ended at or about day of AAV administration (Day 1). ERT was eventually resumed at week 66 following GAA decline.

Methotrexate and Folic Acid Administration. Methotrexate was given in weekly cycles consisting of methotrexate p.o. (orally administered) and folic acid p.o (orally administered). Meaning, methotrexate (to arrest T cell replication and cytotoxic T cell activity) was administered on only day 1. Folic acid (to mitigate toxicity) was given on days 2 through 7 of every cycle. These two drugs are always given in this manner. The dosing plan was methotrexate/folic acid at the highest anticipated dose of methotrexate (30 mg/week) for 12 weeks followed by a tapering regimen where the weekly dose of methotrexate was to be decreased by 5 mg per week/cycle however the dose of folic acid given on days 2 through 7/cycle was maintained at 1 mg regardless of the methotrexate dose until the methotrexate dose reached 0 at which time folic acid was also to be discontinued. To maximize absorption and bioavailability, single doses of methotrexate did not exceed 15 mg, so for higher doses, two doses were given the same day. For example, beginning in week 1, 1 hour prior to administration of vector on day 1, methotrexate was given as 30 mg (15 mg b.i.d.). For the remaining 6 days of the weekly cycle no MTX was given however folic acid 1 mg/day was administered on days 2 through 7. The same was to be followed for 12 weeks (day 1 methotrexate, days 2 through 7 folic acid). Beginning on day 1 of week 13 MTX was to be reduced to 25 mg (15 mg AM, 10 mg PM). Folic acid was given at 1 mg/day for days 2 through 7. Beginning on day 1 of week 14 MTX was to be reduced to 20 mg (10 mg AM, 10 mg PM). Folic acid was to be maintained at 1 mg/day for days 2 through 7. Beginning on day 1 of week 15 methotrexate was to be reduced to 15 mg (10 mg AM, 5 mg PM). Folic acid was to be maintained at 1 mg/day for days 2 through 7. The tapering regimen would continue until the methotrexate dose reached 0 at which time folic acid was also to be discontinued.

Prednisone Administration. The methotrexate/folic acid regimen was given concurrently with a regimen of oral prednisone. The prednisone regimen was the same as in Example 1. Beginning 24 hours prior to vector administration prednisone was given as 60 mg/day for 28 consecutive days. Beginning on day 29 the daily dose was tapered by 5 mg and maintained for 7 days. Thereafter on a 7-day schedule, the daily dose of prednisone was decreased by 5 mg/week, such that on days 1 through 28 prednisone was 60 mg/day, days 29 through 35 prednisone was 55 mg/day, days 36 through 42 prednisone was 50 mg/day etc., until reaching 0 on day 106. On day 110, the prednisone was increased back to 60 mg/day, followed by a more rapid taper down to a dose of 0 after day 144. The regimen of the 2^{nd} taper is shown in Table 9.

TABLE 9
Reactive Prednisone Regimen

| Dose | Duration | Start | Stop |
|---|---|---|---|
| (milligrams p.o.) | (days) | (study day) | (study day) |
| 60 | 7 | 110 | 116 |
| 40 | 7 | 117 | 123 |
| 20 | 7 | 124 | 130 |
| 10 | 7 | 131 | 137 |
| 5 | 7 | 138 | 144 |
| 0 | — | 145 | — |

Prednisone was not restarted throughout the remainder of the study.

### Example 4

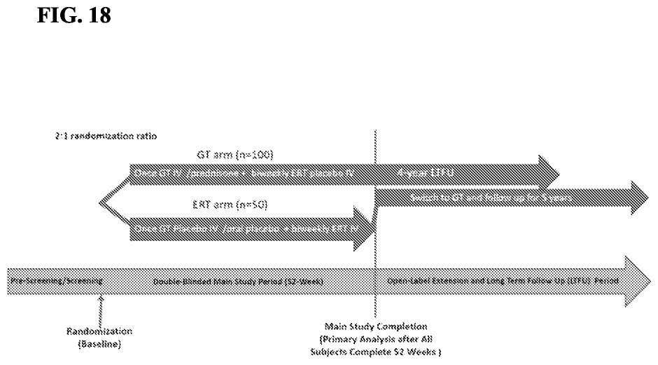

In a double blinded study enrolling patients who have received ERT for at least 52 weeks, subjects (e.g., 150 subjects) will be divided into two groups, a gene therapy arm (GT arm) and a ERT arm (e.g. at a 2:1 ratio). Subjects in the GT arm (e.g., 100 subjects) will receive one dose of gene therapy with the Actus 101 AAV-GAA described in Example 1, by methods described in the above-examples, at a dose of 5E12 vg/kg or less and an additional bi-weekly administration of a placebo rather than ERT. Following the 52 weeks, subjects will be followed up to 5 years. Immune modulator will be administered as needed to suppress immune response to the AAV vector and prevent transaminitis. Subjects in the ERT arm (e.g., 50 subjects) will be administered a one time placebo gene therapy placebo and biweekly ERT for the 52 weeks. Following the 52 week period, subjects will be switched to actual gene therapy to receive as did the subjects in the GT arm, in an open-label extension with long term follow up for up to 5 years. A schematic showing the study design is shown in FIG. 18.

Subjects will be selected and maintained in the study using the criteria of Example 1.

It is expected that the subjects in the GT arm will exhibit similar or better outcome with respect to one or more endpoints including functional endurance (e.g., as measured by the 6MWT and/or FVC), biomarkers such as serum or tissue GAA, tissue glycogen content, health related utility, gross motor function measurements, timed up and go, quality of life, and safety of the therapy.

### REFERENCES

Champiat S, Raposo R A S, Maness N J, Lehman J L, Purtell S E, Hasenkrug A M, et al. (2012) Influence of HAART on Alternative Reading Frame Immune Responses over the Course of HIV-1 Infection. PLoS ONE 7(6): e39311. https://doi.org/10.1371/journal.pone.0039311
Faust S M, Bell P, Cutler B J, et al. CpG-depleted adeno-associated virus vectors evade immune detection. J Clin Invest. 2013; 123(7):2994-3001. doi: 10.1172/JC168205
Han S O, Ronzitti G, Arnson B, et al. Low-Dose Liver-Targeted Gene Therapy for Pompe Disease Enhances Therapeutic Efficacy of ERT via Immune Tolerance Induction [published correction appears in Mol Ther Methods Clin Dev. 2019 May 1; 13:431]. Mol Ther Methods Clin Dev. 2017; 4:126-136. Published 2017 Jan. 11. doi:10.1016/j.omtm.2016.12.010
Han S O, Li S, McCall A, et al. Comparisons of Infant and Adult Mice Reveal Age Effects for Liver Depot Gene Therapy in Pompe Disease. Mol Ther Methods Clin Dev. 2019; 17:133-142. Published 2019 Dec. 6. doi:10.1016/j.omtm.2019.11.020
Kracht, M., van Lummel, M., Nikolic, T. et al. Autoimmunity against a defective ribosomal insulin gene product in type 1 diabetes. Nat Med 23, 501-507 (2017). https://doi.org/10.1038/nm.428
Krieg A M. CpG motifs in bacterial DNA and their immune effects. Annu Rev Immunol. 2002; 20:709-760. doi: 10.1146/annurev.immunol.20.100301.064842
Lorenz F K, Wilde S, Voigt K, et al. Codon optimization of the human papillomavirus E7 oncogene induces a CD8+ T cell response to a cryptic epitope not harbored by wild-type E7. PLoS One. 2015; 10(3):e0121633. Published 2015 Mar. 23. doi:10.1371/journal.pone.0121633
Schirmbeck R, Riedl P, Fissolo N, Lemonnier F A, Bertoletti A, Reimann J. Translation from cryptic reading frames of DNA vaccines generates an extended repertoire of immunogenic, MHC class I-restricted epitopes. J Immunol. 2005; 174(8):4647-4656. doi: 10.4049/jimmunol.174.8.4647
Tóth R, Mészáros I, Hüser D, et al. Methylation Status of the Adeno-Associated Virus Type 2 (AAV2). Viruses. 2019; 11(1):38. Published 2019 Jan. 9. doi:10.3390/v11010038
Yewdell J W, Hickman H D. New lane in the information highway: alternative reading frame peptides elicit T cells with potent antiretrovirus activity. J Exp Med. 2007; 204(11):2501-2504. doi: 10.1084/jem.20071986
Amalfitano, A., et al., 2001. ‘Recombinant human acid alpha-glucosidase enzyme therapy for infantile glycogen storage disease type II: results of a phase I/II clinical trial’, Genet Med, 3: 132-8.
AmericanThoracicSociety. 1991. ‘Lung function testing: selection of reference values and interpretative strategies. American Thoracic Society’, Am Rev Respir Dis, 144: 1202-18.
Angelini, C., et al., 2009. ‘Progress in Enzyme Replacement Therapy in Glycogen Storage Disease Type II’, Ther Adv Neurol Disord, 2: 143-53. ‘ATS statement: guidelines for the six-minute walk test’. 2002. Am J Respir Crit Care Med, 166: 111-7.
Banugaria, S. G., et al., 2011. ‘The impact of antibodies on clinical outcomes in diseases treated with therapeutic protein: lessons learned from infantile Pompe disease’, Genet Med, 13: 729-36.
Brinster, R. L., et al., 1988. ‘Introns increase transcriptional efficiency in transgenic mice’, Proc Natl Acad Sci USA, 85: 836-40.
Brooke, M. H., et al., 1983. ‘Clinical investigation in Duchenne dystrophy: 2. Determination of the “power” of therapeutic trials based on the natural history’, Muscle Nerve, 6: 91-103.
Brooke, M. H., et al., 1987. ‘Clinical investigation of Duchenne muscular dystrophy. Interesting results in a trial of prednisone’, Arch Neurol, 44: 812-7.
Carlier, R. Y., et al., 2011. ‘Whole-body muscle MRI in 20 patients suffering from late onset Pompe disease: Involvement patterns’, Neuromuscul Disord, 21: 791-9.
Crudele, J. M., et al., 2015. ‘AAV liver expression of FIX-Padua prevents and eradicates FIX inhibitor without increasing thrombogenicity in hemophilia B dogs and mice’, Blood, 125: 1553-61.
Daya, S., and K. I. Berns. 2008. ‘Gene therapy using adeno-associated virus vectors’, Clin Microbiol Rev, 21: 583-93.
de Vries, J. M., et al., 2017. ‘Pompe disease in adulthood: effects of antibody formation on enzyme replacement therapy’, Genet Med, 19: 90-97.
Desnick, R. J. 2004. ‘Enzyme replacement and enhancement therapies for lysosomal diseases’, J InheritMetab Dis, 27: 385-410.
Duan, D. 2018. ‘Systemic AAV micro-dystrophin gene therapy for Duchenne Muscular Dystrophy’, Mol Ther, 26: 2337-56.
El-Gharbawy, A. H., et al., 2011. ‘Expanding the clinical spectrum of late-onset Pompe disease: dilated arteriopathy involving the thoracic aorta, a novel vascular phenotype uncovered’, Mol Genet Metab, 103: 362-6.
Flotte, T. R., et al., 2004. ‘Phase I trial of intramuscular injection of a recombinant adeno-associated virus alpha 1-antitrypsin (rAAV2-CB-hAAT) gene vector to AAT-deficient adults’, Hum Gene Ther, 15: 93-128.
Franco, L. M., et al., 2005. ‘Evasion of immune responses to introduced human acid alpha-glucosidase by liver-restricted expression in glycogen storage disease type II’, Mol Ther, 12: 876-84.
Han, S. O., G et al., 2017. ‘Low-Dose Liver-Targeted Gene Therapy for Pompe Disease Enhances Therapeutic Efficacy of ERT via Immune Tolerance Induction’, Mol Ther Methods Clin Dev, 4: 126-36.
Hahn, S. H., et al., 2018. ‘Efficacy, safety profile, and immunogenicity of alglucosidase alfa produced at the 4,000-liter scale in US children and adolescents with Pompe disease: ADVANCE, a phase IV, open-label, prospective study’, Genet Med, 20: 1284-94.
Hobson-Webb, L. D., et al., 2012. ‘Autopsy findings in late-onset Pompe disease: a case report and systematic review of the literature’, Mol Genet Metab, 106: 462-9.
Hundsberger T, Rosler K M, and Findling O. 2014. Cessation and resuming of alglucosidase alfa in Pompe disease: a retrospective analysis. J Neurol. September; 261(9):1684-90.
Ill, C. R., et al., 1997. ‘Optimization of the human factor VIII complementary DNA expression plasmid for gene therapy of hemophilia A’, Blood Coagul Fibrinolysis, 8 Suppl 2: S23-30.
Kishnani, P. S., et al., 2007. ‘Recombinant human acid [alpha]-glucosidase: major clinical benefits in infantile-onset Pompe disease’, Neurology, 68: 99-109.
Kishnani, P. S., et al., 2010. ‘Cross-reactive immunologic material status affects treatment outcomes in Pompe disease infants’, Mol Genet Metab, 99: 26-33.
Koeberl, D. D., et al., 2018. ‘Correction of Biochemical Abnormalities and Improved Muscle Function in a Phase I/II Clinical Trial of Clenbuterol in Pompe Disease’, Mol Ther.
Lachmann, R., and B. Schoser. 2013. ‘The clinical relevance of outcomes used in late-onset Pompe disease: can we do better?’, Orphanet J Rare Dis, 8: 160.
“LUMIZYME Prescribing Information.” In. 2014.
Maga, J. A., et.al., 2013. Glycosylation-independent lysosomal targeting of acid alfa-glucosidase enhances muscle glycogen clearance in pompe mice. J. Biol. Chem. 18 (288): 1428-1438.
Manno, C. S., et al., 2006. ‘Successful transduction of liver in hemophilia by AAV-Factor IX and limitations imposed by the host immune response’, Nat Med, 12: 342-7.
Markusic, D. M., et al., 2013. ‘Effective gene therapy for haemophilic mice with pathogenic factor IX antibodies’, EMBO Mol Med, 5: 1698-709.
Mazaffer, T. 2018. “Updated results from ATB200-02: A first-in-human, open-label, phase 1/2 study of ATB200 co-administered with AT2221 in adults with Pompe disease.” In 14th annual WORLD symposium.
Mendell, J. R., et al., 2017. ‘Single-Dose Gene-Replacement Therapy for Spinal Muscular Atrophy’, N Engl J Med, 377: 1713-22.
Mendell, J. R. et al., 2016. “Gene therapy for spinal muscular atrophy type 1 shows potential to improve survival and motor funtional outcomes.” In ASGCT; 19th Annual Meeting., S190.
Mendelsohn, N. J., Y. H. Messinger, A. S. Rosenberg, and P. S. Kishnani. 2009. ‘Elimination of antibodies to recombinant enzyme in Pompe's disease’, N Engl J Med, 360: 194-5.
Moxley, R. T., 3rd. 1990. ‘Functional testing’, Muscle Nerve, 13 Suppl: S26-9.
Nathwani, A. C., et al., 2014. ‘Long-term safety and efficacy of factor IX gene therapy in hemophilia B’, N Engl J Med, 371: 1994-2004.
Nathwani, A. C., C et al., 2011. ‘Long-term safety and efficacy following systemic administration of a self-complementary AAV vector encoding human FIX pseudotyped with serotype 5 and 8 capsid proteins’, Mol Ther, 19: 876-85.
Nathwani, A. C., et al., 2011. ‘Adenovirus-associated virus vector-mediated gene transfer in hemophilia B’, N Engl J Med, 365: 2357-65.
Niemeyer, G. P., et al., 2009. ‘Long-term correction of inhibitor-prone hemophilia B dogs treated with liver-directed AAV2-mediated factor IX gene therapy’, Blood, 113: 797-806.
Patel, T. T., S. G. Banugaria, L. E. Case, S. Wenninger, B. Schoser, and P. S. Kishnani. 2012. ‘The impact of antibodies in late-onset Pompe disease: a case series and literature review’, Mol Genet Metab, 106: 301-9.
Ponder, K. P. 2011. ‘Merry christmas for patients with hemophilia B’, N Engl J Med, 365: 2424-5.
Raben, N., et al., 2003. ‘Enzyme replacement therapy in the mouse model of Pompe disease’, Mol Genet Metab, 80: 159-69.
Raj, D., A. M. Davidoff, and A. C. Nathwani. 2011. ‘Self-complementary adeno-associated viral vectors for gene therapy of hemophilia B: progress and challenges’, Expert Rev Hematol, 4: 539-49.
Rangarajan, S., et al., 2017. ‘AAV5-Factor VIII Gene Transfer in Severe Hemophilia A’, N Engl J Med, 377: 2519-30.
Russell, D. J., et al., 1989. The gross motor function measure: a means to evaluate the effects of physical therapy. Dev. Med. Child. Neurol 31: 341-352.
Scheidegger O, et al., 2018. 36-Months follow-up assessment after cessation and resuming of enzyme replacement therapy in late onset Pompe disease: data from the Swiss Pompe Registry. J Neurol. 265 (12):2783-2788. doi: 10.1007/s00415-018-9065-7.
Schoser, B., A. et al., 2017. ‘Survival and long-term outcomes in late-onset Pompe disease following alglucosidase alfa treatment: a systematic review and meta-analysis’, J Neurol, 264: 621-30.
Schwarts, C. E., V. E. Powell, and A. Eldar-Lissai. 2017. ‘Measuring hemophilia caregiver burden: validation of the Hemophilia Caregiver Impact measure’, Qual Life Res, 26: 2551-62.
“Summary of clinical trials using AAV gene transfer.” In. 2015.
Sun, B., et al., 2007. ‘Enhanced response to enzyme replacement therapy in Pompe disease after the induction of immune tolerance’, Am J Hum Genet, 81: 1042-9.
Sun, B., et al., 2010. ‘Immunomodulatory gene therapy prevents antibody formation and lethal hypersensitivity reactions in murine pompe disease’, Mol Ther, 18: 353-60.
Sun, B., et al., 2006. ‘Enhanced efficacy of an AAV vector encoding chimeric, highly secreted acid alpha-glucosidase in glycogen storage disease type II’, Mol Ther, 14: 822-30.
Tarnopolsky, M., et al., 2016. ‘Pompe Disease: Diagnosis and Management. Evidence-Based Guidelines from a Canadian Expert Panel’, Can J Neurol Sci, 43: 472-85.
van Capelle, C. I., N. A. et al., 2012. ‘The quick motor function test: a new tool to rate clinical severity and motor function in Pompe patients’, J Inherit Metab Dis, 35: 317-23.
van Capelle, C. I., N. A. et al., 2010. ‘Effect of enzyme therapy in juvenile patients with Pompe disease: a three-year open-label study’, Neuromuscul Disord, 20: 775-82.
van der Beek, N. A., C. I. et al., 2011. ‘Rate of progression and predictive factors for pulmonary outcome in children and adults with Pompe disease’, Mol Genet Metab, 104: 129-36.
van der Ploeg, A. T., et al., 2010. ‘A randomized study of alglucosidase alfa in late-onset Pompe's disease’, N Engl J Med, 362: 1396-406.
Vercauteren, K., et al., 2016. ‘Superior in vivo transduction of human hepatocytes using engineered AAV3 capsid.’, Mol Ther, 24: 1042-49.
Ziegler, R. J., et al., 2008. ‘Ability of adeno-associated virus serotype 8-mediated hepatic expression of acid alpha-glucosidase to correct the biochemical and motor function deficits of presymptomatic and symptomatic Pompe mice’, Hum Gene Ther, 19: 609-21.

While the present inventions have been described and illustrated in conjunction with a number of specific embodiments, those skilled in the art will appreciate that variations and modifications may be made without departing from the principles of the inventions as herein illustrated, as described and claimed. The present inventions may be embodied in other specific forms without departing from their spirit or essential characteristics. The described embodiments are considered in all respects to be illustrative and not restrictive.

In closing, regarding the exemplary embodiments of the present invention as shown and described herein, it will be appreciated that a genomic construct, comprising an AAV (adeno-associated virus) viral virion is disclosed and configured for delivery of AAV vectors. Because the principles of the invention may be practiced in a number of configurations beyond those shown and described, it is to be understood that the invention is not in any way limited by the exemplary embodiments, but is generally directed to a genomic construct, comprising an AAV (adeno-associated virus) viral virion apparatus and is able to take numerous forms to do so without departing from the spirit and scope of the invention.

Certain embodiments of the present invention are described herein, including the best mode known to the inventor(s) for carrying out the invention. Of course, variations on these described embodiments will become apparent to those of ordinary skill in the art upon reading the foregoing description. The inventor(s) expect skilled artisans to employ such variations as appropriate, and the inventor(s) intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein. Similarly, as used herein, unless indicated to the contrary, the term “substantially” is a term of degree intended to indicate an approximation of the characteristic, item, quantity, parameter, property, or term so qualified, encompassing a range that can be understood and construed by those of ordinary skill in the art.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

When used in the claims, whether as filed or added per amendment, the open-ended transitional term “comprising” (along with equivalent open-ended transitional phrases thereof such as “including,” “containing” and “having”) encompasses all the expressly recited elements, limitations, steps and/or features alone or in combination with un-recited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amendment for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim, whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (along with equivalent open-ended transitional phrases thereof) includes within its meaning, as a limiting case, claimed subject matter specified by the closed-ended transitional phrases “consisting of” or “consisting essentially of” As such, embodiments described herein or so claimed with the phrase “comprising” are expressly or inherently unambiguously described, enabled and supported herein for the phrases “consisting essentially of” and “consisting of.”

While aspects of the invention have been described with reference to at least one exemplary embodiment, it is to be clearly understood by those skilled in the art that the invention is not limited thereto. Rather, the scope of the invention is to be interpreted only in conjunction with the appended claims and it is made clear, here, that the inventor(s) believe that the claimed subject matter is the invention.

The references disclosed in the specification and Examples, including but not limited to patents and patent applications, and international patent applications are all incorporated herein in their entirety by reference.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. A method of treating Pompe disease in a subject, comprising administering to a subject who is being treated for Pompe disease with long-term GAA enzyme replacement therapy (ERT), a pharmaceutical composition comprising a recombinant adeno-associated virus (AAV) vector comprising in its genome, a heterologous nucleic acid sequence encoding a polypeptide comprising an alpha-glucosidase (GAA) polypeptide in expressible form at a dosage no more than 4.0E¹² vg/kg, wherein the heterologous nucleic acid is operatively linked to a liver-specific promoter, and wherein the administration of long-term GAA enzyme replacement therapy (ERT) is withdrawn by at least 26 weeks post administration of the recombinant AAV, and wherein the subject obtains a blood serum level of GAA expressed by the AAV at a pharmacological activity is at least 165 nmol/ml/hr of at least within two weeks of administration.

2. (canceled)

3. The method of claim 1, wherein the subject has previously undergone long-term administration of ERT for Pompe disease.

4. The method of claim 3, wherein the administration of ERT in the subject is withdrawn concurrently or prior to the administration of the AAV vector.

5. (canceled)

6. The method of claim 3, wherein the administration of ERT is withdrawn after the administration of the AAV vector.

7. The method of claim 6, wherein the administration of ERT is withdrawn during a period of about day 0 of the administration to about 26 weeks after the administration of the AAV vector.

8. (canceled)

9. The method of claim 1, further comprising administering complementary ERT after an extended period of time following the administration of the AAV vector and further following withdrawal of the administration of the long term ERT, wherein the extended period of time is at least 1 year following the administration of the AAV vector.

10.-13. (canceled)

14. The method of claim 9, wherein the complementary ERT is administered at a lower frequency and/or dosage than the administration of the long-term ERT.

15.-16. (canceled)

17. The method of claim 14, wherein the complementary ERT administration is sporadic.

18. The method of claim 1, wherein the pharmaceutical composition comprises a dose of rAAV of between 1.6E12 vg/kg and 3.2E12 vg/kg.

19. The method of claim 17, wherein the dose of rAAV is sufficient to express GAA to achieve clinical stability in the subject in one or more symptoms of Pompe disease, wherein clinical stability is selected from the group consisting of a) not exceeding a 15% decrease in Forced Vital Capacity (FVC) over two consecutive assessments, measured no less than 3-months apart, b) not exceeding a 12% decrease in the 6MWT over two consecutive assessment, measured no less than 3-months apart, and c) not exceeding a 43-meter decrease in the 6MWT over two consecutive assessments, measured no less than 3-months apart.

20. The method of claim 1, wherein the pharmaceutical composition comprises a dose of rAAV sufficient to achieve serum level of hGAA expressed by the rAAV at a pharmacological activity range from at least about 165 to ≤2,260 nmol/ml/hr.

21.-25. (canceled)

26. The method of claim 1, wherein the nucleic acid sequence encoding the GAA polypeptide is the human GAA gene or a human codon optimized GAA gene (coGAA) or a modified GAA nucleic acid sequence.

27. The method of claim 1, wherein the nucleic acid sequence encoding the GAA polypeptide encodes a GAA polypeptide which comprises at least one, at least 2 or at least all three amino acid modifications selected from; H201L, H199R, R233H, V780I or V780R of SEQ ID NO: 10.

28. The method of claim 1, wherein the heterologous nucleic acid sequence encodes a GAA polypeptide having the amino acid sequence of SEQ ID NO: 600 or a functional variant or functional fragment thereof having at least 90%, 95% or 99% activity to SEQ ID NO: 600.

29.-31. (canceled)

32. The method of claim 31, wherein one or more CpG islands in the ITR are removed.

33.-35. (canceled)

36. The method of claim 1, wherein the AAV3b serotype comprises one or more mutations in a capsid protein selected from any of: 265D, 549A, Q263Y.

37. The method of claim 36, wherein the capsid serotype is selected from any of: AAV3b265D, AAV3b265D549A, AAV3b549A, AAV3bQ263Y, AAV3bSASTG, AAV8, AAVXL32, of AAVXL32.1.

38.-39. (canceled)

40. The method of claim 1, further comprising administering to the subject an immunosuppressant at a first dose for a first time period, the first time period beginning the day before, or on the day of the administration of the AAV vector.

41. The method of claim 1, further comprising administering to the subject an immunosuppressant at incremental tapering doses, each incremental tapering dose is administered for a defined period of time after the first time period of administration of the immunosuppressant at the first dose.

42.-48. (canceled)

49. The method of claim 1, wherein T cell reactivity to vector capsid is below a threshold range of >40 to <129/sfu/million.

50. A method of reducing or eliminating the clinical need for GAA enzyme replacement therapy (ERT) in a subject with Pompe disease, comprising or consisting essentially of, administering to the subject a pharmaceutical composition comprising a recombinant adeno-associated virus (AAV) vector comprising in its genome, a heterologous nucleic acid sequence encoding a polypeptide comprising an alpha-glucosidase (GAA) polypeptide in expressible form at a dosage no more than 4.0E¹² vg/kg, wherein the heterologous nucleic acid is operatively linked to a liver-specific promoter, and wherein the subject obtains a blood serum level of GAA expressed by the AAV at a pharmacological activity range from 165 to ≤2,260 nmol/ml/hr of at least within two weeks of administration.

51. The method of claim 50, wherein the frequency of administration of the GAA ERT and/or dosage of GAA ERT administered is reduced by at least 20% or at least 50% in the first year after administration of the AAV vector as compared to the year prior to said administration, without substantial clinical decline.

52.-54. (canceled)

55. The method of claim 50, wherein GAA activity in muscle is increased by at least 2 fold than the level before the AAV vector was administered.

56.-57. (canceled)

58. A method of treating Pompe disease in a subject, comprising administering to a subject who is being treated for Pompe disease with long-term GAA enzyme replacement therapy (ERT), a pharmaceutical composition comprising a recombinant adeno-associated virus (AAV) vector comprising in its genome, a heterologous nucleic acid sequence encoding a polypeptide comprising an alpha-glucosidase (GAA) polypeptide in expressible form at a dosage of between about 1.6 e¹² vg/kg to about 1.6e¹³ vg/kg, wherein the heterologous nucleic acid is operatively linked to a liver-specific promoter, and wherein the administration of long-term GAA enzyme replacement therapy (ERT) is withdrawn on the same day (d1), the day after, or at least the day before the administration of the recombinant AAV, and wherein the subject obtains a blood serum level of GAA expressed by the AAV at a pharmacological activity is at least 165 nmol/ml/hr of at least within two weeks of administration.

59. The method of claim 58, wherein the subject is administered methotrexate, prednisone, or a combination of both, for immune modulation.

60. The method of claim 59, where methotrexate is administered at an initial dose of 30 mg or less/week.

61. The method of claim 60, wherein methotrexate is administered at an initial dose of between about 5 and 30 mg/week.

62. The method of claim 61, wherein the methotrexate is administered at an initial dose of between 5 and 7.5 mg/week.
